(19) United States
(12) Patent Application Publication
(10) Pub. No.: US 2023/0405116 A1
(43) Pub. Date: Dec. 21, 2023

(54) VECTORS, SYSTEMS AND METHODS FOR EUKARYOTIC GENE EDITING

(71) Applicant: Wake Forest University Health Sciences, Winston-Salem, NC (US)

(72) Inventors: Baisong LU, Winston-Salem, NC (US); Anthony ATALA, Winston-Salem, NC (US)

(73) Assignee: Wake Forest University Health Sciences, Winston-Salem, NC (US)

(21) Appl. No.: 18/037,708

(22) PCT Filed: Nov. 19, 2021
(86) PCT No.: PCT/US2021/060099

§ 371 Date: May 18, 2023

Related U.S. Application Data
(60) Provisional application No. 63/115,932, filed on Nov. 19, 2020.

(51) Int. Cl. A61K 39/00; C12N 15/11; C12N 9/22; C12N 9/78; C12N 15/86; A61K 48/00

(52) U.S. Cl. CPC A61K 39/4611; C12N 15/11; C12N 9/22; C12N 9/78; C12Y 305/04004; C12N 15/86; A61K 48/0041; C12N 2310/20; C12N 2310/16; C12N 2310/531

(57) ABSTRACT

Provided herein are compositions and methods for editing the genome of a eukaryotic cell.

### PRIOR RELATED APPLICATION

This application claims the benefit of U.S. Provisional Application No. 63/115,932 filed on Nov. 19, 2020, which is hereby incorporated by reference in its entirety.

### FIELD

This disclosure describes compositions and methods of using same for eukaryotic gene editing.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 095199-1275954_seqlist, created on Nov. 15, 2021, and having a size of 79.0 kb and is filed concurrently with the specification. The sequence listing contained in this ASCII formatted document is part of the specification and is herein incorporated by reference in its entirety.

### BACKGROUND

Fusion of adenine deaminases to nuclease-deficient type CRISPR/Cas9 (clustered regularly interspaced short palindromic repeats/CRISPR-associated 9) creates adenine base editors (ABEs) that can edit genomic DNA without double-stranded DNA cleavage. Base editing generates precise point mutations in genomic DNA without generating double strand breaks. Further, adenine base editing does not require a DNA donor template and does not rely on cellular homologous directed repair. Thus, it has great potential as a gene therapy for genetic diseases caused by transition mutations, which account for 61% of disease-causing point mutations. Although Adenine base editors (ABEs) have been used in many in vitro and in vivo studies, ABEs have shown significant guide-independent RNA off-target activities that raise safety concerns and hinder their potential clinical applications. Thus, compositions and methods for reducing RNA off-target activities of ABEs are necessary.

### SUMMARY

Provided herein is a mammalian expression plasmid comprising a eukaryote, promoter operably linked to a non-viral nucleic acid sequence, wherein the non-viral nucleic acid sequence comprises: (i) a nucleic acid sequence encoding an adenosine base pair editor (ABE), wherein the ABE is a fusion protein comprising an adenosine deaminase and a catalytically impaired CRISPR-associated endonuclease; and (ii) a guide RNA (gRNA) coding sequence, wherein the gRNA coding sequence comprises at least one aptamer coding sequence.

In some embodiments, the catalytically impaired CRISPR-associated endonuclease coding sequence encodes a Cas9 D10A protein. In some embodiments, the adenine base editor is ABE7.10 or ABE8. In some embodiments, the at least one aptamer coding sequence encodes an aptamer sequence bound specifically by an ABP selected from the group consisting of MS2 coat protein, PP7 coat protein, lambda N RNA-binding domain, or Corn protein. In some embodiments, the aptamer is an MS2 aptamer sequence or a corn aptamer sequence. In some embodiments, the sgRNA coding sequence comprises at least one aptamer inserted into the tetraloop or the ST2 loop of the sgRNA coding sequence. In some embodiments, the sgRNA coding comprises at least one corn aptamer inserted into the ST2 loop of the gRNA coding sequence.

Also provided is a lentiviral packaging system comprising: (a) a packaging plasmid comprising a eukaryotic promoter operably linked to a Gag nucleotide sequence, wherein the Gag nucleotide sequence comprises a nucleocapsid (NC) coding sequence and a matrix protein (MA) coding sequence, wherein one or both of the NC coding sequence or the MA coding sequence comprises at least one non-viral aptamer-binding protein (ABP) nucleotide sequence, and wherein the packaging plasmid does not encode a functional integrase protein; (b) at least one mammalian expression plasmid provided herein; and (c) an envelope plasmid comprising an envelope glycoprotein coding sequence.

In some embodiments, the packaging plasmid further comprises a Rev nucleotide sequence and a Tat nucleotide sequence. In some embodiments, the system further comprises a second packaging plasmid comprising a Rev nucleotide sequence. In some embodiments, the at least one non-viral ABP nucleotide sequence encodes MS2 coat protein, PP7 coat protein, lambda N peptide, or Com protein.

Further provided is a lentivirus-like particle comprising: (a) a fusion protein comprising a nucleocapsid (NC) protein or a matrix (MA) protein wherein the NC protein or MA protein comprises at least one non-viral aptamer binding protein (ABP); and (b) ribonucleotide protein (RNP) complex comprising: (i) an adenine base editor (ABE), wherein the ABE is a fusion polypeptide comprising an adenine base editor and a catalytically impaired CRISPR-associated endonuclease; and (ii) a gRNA, wherein the lentivirus-like particle does not comprise a functional integrase protein. In some lentivirus-like particle, the catalytically impaired CRISPR-associated endonuclease is a catalytically impaired Cas9 protein, a catalytically impaired Cpf1 protein, or a derivative of either. In some lentivirus-like particles, the adenine base editor is ABE 7.10 or ABE 8.

Also provided is a method of producing a lentivirus-like particle, the method comprising: (a) transfecting a plurality of eukaryotic cells with the packaging plasmid, the at least one mammalian expression plasmid, and the envelope plasmid of any of the systems described herein; and (h) culturing the transfected eukaryotic cells for sufficient time for lentivirus-like particles to be produced. In some embodiments, the lentivirus-like particle produced comprises a RNP comprising: (i) an adenine base editor (ABE), wherein the ABE is a fusion polypeptide comprising an adenosine deaminase and a catalytically impaired CRISPR-associated endonuclease; and (ii) a guide RNA. In some embodiments, the plurality of eukaryotic cells are mammalian cells.

Further provided is a method of modifying a genomic target sequence in a cell, the method comprising transducing a plurality of eukaryotic cells with a plurality of viral particles described herein, wherein the RNP binds to the genomic target sequence in genomic DNA of the cell and the ABE deaminates an adenine at the genomic target sequence, thereby modifying the genomic target sequence. In some methods, the plurality of eukaryotic cells are mammalian cells. In some embodiments, the plurality of eukaryotic cells are cells present in subject. In some embodiments, the subject is a human subject. In some embodiments, the subject is injected with the plurality of viral particles.

Also provided are cells comprising any of the plasmids, lentiviral packaging systems or lentivirus-like particles described herein. Cells modified by any of the methods provided herein are also provided.

Further provided is a method for treating a disease in a subject comprising: (a) obtaining cells from the subject; and (b) modifying the cells of the subject using any of the genomic editing methods described herein; and administering the modified cells to the subject. In some embodiments, the disease is cancer. In some embodiments, the disease is sickle cell anemia. In some embodiments, the cells are T cells.

### DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1A is a diagram showing the predicted ABE off-target hotspot in human USP38 mRNA according to aspects of this disclosure. The predicted hotspot (red) and the primers used for PCR amplification are indicated.

FIG. 1B shows the results of RT-PCR and targeted NGS which detected high levels of A to G changes in a 440 nt region of USP38 mRNA region after ABE DNA transfection according to aspects of this disclosure. The peaks above the X-axis were observed in cells transfected with plasmid DNA expressing ABE and sgRNA targeting ABE-site 1. The peaks (very low, in the negative area) were observed in control cells (transfected with Cas9 nickase targeting ABE-site 1). The highest peak corresponding to the predicted hotspot (CUACGAA) is indicated.

FIG. 1C shows the sequences of the most frequent NGS reads (SEQ ID NOs: 108-117) from cells transfected with plasmid DNA expressing ABE targeting ABE-site 1 according to aspects of this disclosure. The predicted RNA off-target hotspot is underlined (highest peak). The A to G changes are shown. The TA dinucleotide marked by a dashed box corresponds to the second peak marked in FIG. 1B. The three shaded alleles do not have A to G changes in the hotspot but have A to G changes in the second peak. DNA samples were collected 48 hours after treatment.

FIG. 1D. shows the results of next generation sequence (NGS) analysis of on-target base editing at ABE site 1 according to aspects of this disclosure. SEQ ID NO: 118 is shown as a Reference sequence. 96.30% of the reads corresponded to SEQ ID NO: 118, with SEQ ID NOs: 119 and 120 representing 2.22% and 0.25% of the reads, respectively. Shown are data from cells (2×10⁵) treated with 20 μg ABE RNPs and collected 24 hours after electroporation for NGS.

FIG. 2A is an exemplary modification to an sgRNA scaffold for ABE RNP packaging according to aspects of this disclosure (SEQ ID NO: 121). The Tetraloop (GAAA) and the ST2 loop are indicated by dashed boxes. The core aptamer sequences are underlined and the additional linkers are not underlined. Vertical lines indicate complementary base pairs and dots indicate non-canonical base pairs. As shown in FIG. 3A, the tetraloop and the ST2 loop can be replaced with an MS2 aptamer sequence (SEQ ID NO: 122). In another example, the tetraloop or the ST2 loop can be replaced with a corn aptamer sequence (SEQ ID NO: 123).

FIG. 2B. shows the results of qPCR to detect ABE-g1 RNP activity on ABE site 1 according to aspects of this disclosure. A total of 200 ng p24 of various LV capsids were used to transduce 2.5×10⁴ HEK293T cells. The gDNA was used for qPCR with primers matching edited sequences. *** indicates p<0.0001, Tukey's multiple comparison test following one-way analysis of variance (ANOVA). Error bars indicate s.e.m, of three replicates.

FIG. 2C shows the results of qPCR to detect ABE-g5 RNP activity on ABE site 5 according to aspects of this disclosure. * p<0.05, ns=not significant; Tukey's multiple comparison test following ANOVA.

FIG. 2D shows NGS analysis of capsid-RNP-mediated base editing at ABE site 5 according to aspects of this disclosure. Capsid-RNPs (108 ng p24) were used to transduce 2.5×10⁴ HEK293T cells. SEQ ID NO: 124 is a reference sequence Alleles with base editing frequencies of >0.2% are listed (SEQ ID NOs: 125-133) and frequencies with A>G changes at different positions are shown at the bottom.

FIG. 3 shows NGS analysis of capsid-RNP mediated base editing at ABE site 1 according to aspects of this disclosure. Capsid-RNPs in the amount of 200 ng p24 were used to transduce 2.5×10⁴ HEK293T cells. SEQ ID NO: 134 is a Reference sequence. The alleles with base editing frequencies of >0.1% were listed (SEQ ID NOs: 134-139) and the frequencies with A>G changes at different positions are shown at the bottom.

FIG. 4A shows that aptamer/(aptamer binding protein (ABP) interaction is necessary for functional ABE packaging in lentiviral capsids according to aspects of this disclosure. Forty ng p24 (ELISA) ABE-g5 RNP capsids and ABE-g5^{ST2-com} RNP capsids were treated with or without Triton™-X100, p24 and ABE were detected by western blotting. The p24 images were from the same blot with non-relevant lanes removed. Asterisks indicate the full-length protein.

FIG. 4B. shows estimates of ABE protein amounts in LV capsids according to aspects of this disclosure.

FIG. 4C shows the results of qPCR detection of base editing activities of ABE-g5 RNP capsids and ABE-g5^{ST2-com} RNP capsids according to aspects of this disclosure. 2.5×10⁴ HEK293T cells were treated with 200 ng p24 of capsids-RNPs. 48 hours later gDNA was extracted and analyzed by qPCR to detect base editing at site 5. DNA from cells treated with ABE-g1^{ST2-com} RNP capsids (from FIG. 3B) was used as the control to show site specificity.

FIG. 4D shows the results of qPCR using known concentrations of plasmid DNA to examine the effects of com addition on PCR detection according to aspects of this disclosure.

FIG. 4E shows RT-qPCR comparison of sgRNA levels in ABE-g5 RNP and ABE-g5^{ST2-com} RNP capsids treated with and without Triton™ X-100 according to aspects of this disclosure. *** indicates p<0.0001 in Bonferroni post hoc tests following ANOVA.

FIG. 5A is a Western blot of ABE levels after transducing HEK293T cells according to aspects of this disclosure. Gel images of ABE and β-actin are shown. The arrow indicates position of the full-length ABE bands. The β-actin image demonstrates that all samples have lysate input. Normalization was not attempted since the RNP amount was independent of cell proliferation.

FIG. 5B is a densitometry analysis of protein degradation according to aspects of this disclosure. Only the full-length ABE band was quantified. Half-life was estimated using the two-phase decay model in GraphPad Prism 5.0.

FIG. 6 is an NGS analysis of RNA off-targets in capsid-RNP treated cells at the hotspot in USP38 mRNA according to aspects of this disclosure. Substitution rates in capsid-RNP (targeting ABE site 1) treated cells (peaks above the X-axis) and in negative control cells treated with nickase (peaks below the X-axis) showed no difference. A to G change rates at both peaks were of background level. The position of the predicted hotspot is indicated. Shown is a representative picture of one of the two experiments.

### DEFINITIONS

As used in this specification and the appended claims, the singular forms “a,” “an,” and “the” include plural reference unless the context clearly dictates otherwise.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The term “nucleic acid” or “nucleotide” refers to deoxyribonucleic acids (DNA) or ribonucleic acids (RNA) and polymers thereof in either single- or double-stranded form. It is understood that when an RNA is described, its corresponding DNA is also described, wherein uridine is represented as thymidine. Similarly, when a DNA is described, its corresponding RNA is also described wherein thymidine is represented by uridine. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The polynucleotides of the invention also encompass all forms of sequences including, but not limited to, single-stranded forms, double-stranded forms, hairpins, stem-and-loop structures, and the like.

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA, or micro RNA.

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds, lentivirus-like particles or agents of the present disclosure to prevent or delay, to alleviate, or to arrest or inhibit development of the symptoms or conditions associated with a disease, condition or disorder as described herein. The term “therapeutic effect” refers to the reduction, elimination, or prevention of the disease, symptoms of the disease, or side effects of the disease in the subject. “Treating” or “treatment” using the methods of the present disclosure includes preventing the onset of symptoms in a subject that can be at increased risk of a disease or disorder associated with a disease, condition or disorder as described herein, but does not yet experience or exhibit symptoms, inhibiting the symptoms of a disease or disorder (slowing or arresting its development), providing relief from the symptoms or side effects of a disease (including palliative treatment), and relieving the symptoms of a disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition. The term “treatment,” as used herein, includes preventative (e.g., prophylactic), curative, or palliative treatment.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass full-length proteins, truncated proteins, and fragments thereof, and amino acid chains, wherein the amino acid residues are linked by covalent peptide bonds. As used throughout, the term “fusion polypeptide” or “fusion protein” is a polypeptide comprising two or more proteins or fragments thereof. In some embodiments, a linker comprising about 3 to 10 amino acids can be positioned between any two proteins or fragments thereof to help facilitate proper folding of the proteins upon expression.

The term “identity” or “substantial identity”, as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. It is understood that sequences having at 60%, 65%, 70%, 75%, 80%, 85%, 90%, 91%, 92%, 93%, 94%, 95%, 96%, 97%, 98%, or 99% identity to any nucleotide or polypeptide sequence set forth herein, for example, any one of SEQ ID NOs: 1-48, can be used in the compositions and methods provided herein. It is understood that a nucleic acid sequence can comprise, consist of, or consist essentially of any nucleic acid sequence described herein. Similarly, a polypeptide can comprise, consist of, or consist essentially of, any polypeptide sequence described herein. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, about 20 to 50, about 20 to 100, about 50 to about 200 or about 100 to about 150, in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990)J Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

As used throughout, by subject is meant an individual. For example, the subject is a mammal, such as a primate, and, more specifically, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations are contemplated herein. The term does not denote a particular age or sex. Thus, adult and newborn subjects, whether male or female, are intended to be covered. As used herein, patient or subject may be used interchangeably and can refer to a subject afflicted with a disease or disorder.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter, followed by a transcription termination signal sequence. An expression cassette may or may not include specific regulatory sequences, such as 5′ or 3′ untranslated regions from human globin genes.

A “reporter gene” encodes proteins that are readily detectable due to their biochemical characteristics, such as enzymatic activity or chemifluorescent features. These reporter proteins can be used as selectable markers. One specific example of such a reporter is green fluorescent protein. Fluorescence generated from this protein can be detected with various commercially-available fluorescent detection systems. Other reporters can be detected by staining. The reporter can also be an enzyme that generates a detectable signal when contacted with an appropriate substrate. The reporter can be an enzyme that catalyzes the formation of a detectable product. Suitable enzymes include, but are not limited to, proteases, nucleases, lipases, phosphatases and hydrolases. The reporter can encode an enzyme whose substrates are substantially impermeable to eukaryotic plasma membranes, thus making it possible to tightly control signal formation. Specific examples of suitable reporter genes that encode enzymes include, but are not limited to, CAT (chloramphenicol acetyl transferase; Alton and Vapnek (1979) Nature 282: 864-869); luciferase (lux); β-galactosidase; LacZ; β-glucuronidase; and alkaline phosphatase (Toh, et al. (1980) Eur. J. Biochem. 182: 231-238; and Hall et al. (1983) J. Mol. Appl. Gen. 2: 101), each of which are incorporated by reference herein in its entirety. Other suitable reporters include those that encode for a particular epitope that can be detected with a labeled antibody that specifically recognizes the epitope.

In the compositions and methods provided herein, the CRISPR-associated endonuclease is a catalytically impaired nuclease. As used throughout, “catalytically impaired” refers to decreased CRISPR-associated endonuclease enzymatic activity for cleaving one or both strands of DNA. Examples of catalytically impaired CRISPR-associated endonucleases include but are not limited to catalytically impaired Cas9, catalytically impaired Cpf1 and catalytically impaired C2c2. In some instances, the catalytically impaired CRISPR-associated endonuclease is a the catalytically impaired Cas9, for example Cas9 D10A, which cleaves or nicks only one strand of DNA. In some instances, the CRISPR-associated endonuclease may be a catalytically impaired CRISPR-associated endonuclease, wherein the endonuclease cannot cleave both strands of a double-stranded DNA molecule, i.e., cannot make a double-stranded break. Modifications include, but are not limited to, altering one or more amino acids to inactivate the nuclease activity or the nuclease domain. For example, and not to be limiting, D10A and/or H840A mutations can be made in Cas9 from Streptococcus pyogenes to reduce or inactivate Cas9 nuclease activity. Other modifications include removing all or a portion of the nuclease domain of Cas9, such that the sequences exhibiting nuclease activity are absent from Cas9. Accordingly, a catalytically impaired Cas9 may include polypeptide sequences modified to reduce nuclease activity or removal of a polypeptide sequence or sequences to reduce nuclease activity. The catalytically impaired Cas9 retains the ability to bind to DNA even though the nuclease activity has been inactivated. Accordingly, a catalytically impaired Cas9 includes the polypeptide sequence or sequences required for DNA binding but includes modified nuclease sequences or lacks nuclease sequences responsible for nuclease activity. It is understood that similar modifications can be made to reduce nuclease activity in other site-directed nucleases, for example in Cpf1 or C2c2. In some examples, the Cas9 protein is a full-length Cas9 sequence from S. pyogenes lacking the polypeptide sequence of the RuvC nuclease domain and/or the HNH nuclease domain and retaining the DNA binding function. In other examples, the Cas9 protein sequences have at least 30%, 40%, 50%, 60%, 70%, 80%, 90%, 95%, 98% or 99% identity to Cas9 polypeptide sequences lacking the RuvC nuclease domain and/or the HNH nuclease domain and retains DNA binding function.

Examples of CRISPR-associate endonucleases that can be catalytically impaired include, but are not limited to, nucleases present in any bacterial species that encodes a Type II or a Type V CRISPR/Cas system. The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. The CRISPR/Cas system classification as described in by Makarova, et al. (Nat Rev Microbiol. 2015 November; 13(11):722-36) defines five types and 16 subtypes based on shared characteristics and evolutionary similarity. These are grouped into two large classes based on the structure of the effector complex that cleaves genomic DNA. The Type II CRISPR/Cas system was the first used for genome engineering, with Type V following in 2015. Wild-type type II CRISPR/Cas systems utilize an RNA-mediated nuclease Cas protein or homolog (referred to herein as a “CRISPR-associated endonuclease”) in complex with guide RNA to recognize and cleave foreign nucleic acid. Cas9 proteins also use an activating RNA (also referred to as a transactivating or tracr RNA). Guide RNAs having the activity of either a guide RNA or both a guide RNA and an activating RNA, depending on the type of CRISPR-associated endonuclease used therewith, are also known in the art. In some cases, such dual activity guide RNAs are referred to as a single guide RNA (sgRNA). Synthetic guide RNAs that do not contain an activating RNA sequence may also be referred to as sgRNAs. In this disclosure, the terms sgRNA and gRNA are used interchangeably to refer to an RNA molecule that complexes with a CRISPR-associated endonuclease and localizes the ribonucleoprotein complex to a target DNA sequence.

For example, and not to be limiting, the CRISPR-associated endonuclease can be a Cas9 polypeptide (Type II) or a Cpf1 polypeptide (Type V). See, for example, Abudayyeh et al., Science 2016 Aug. 5; 353(6299):aaf5573; Fonfara et al. Nature 532: 517-521 (2016), and Zetsche et al., Cell 163(3): p. 759-771, 22 Oct. 2015. As used throughout, the term “Cas9 polypeptide” means a Cas9 protein, or a fragment or derivative thereof, identified in any bacterial species that encodes a Type II CRISPR/Cas system. See, for example, Makarova et al. Nature Reviews, Microbiology, 9: 467-477 (2011), including supplemental information, hereby incorporated by reference in its entirety. CRISPR-associated endonucleases, such as Cas9 and Cas9 homologs, are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein (SpCas9). Another exemplary Cas9 protein is the Staphylococcus aureus Cas9 protein (SaCas9). Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. The Cas9 nuclease domains can be optimized for efficient activity or enhanced stability in the host cell. Other CRISPR-associated endonucleases include Cpf1 (See, e.g., Zetsche et al., Cell, Volume 163, Issue 3, p. 759-771, 22 Oct. 2015) and homologs thereof.

Full-length Cas9 is an endonuclease comprising a recognition domain and two nuclease domains (HNH and RuvC, respectively) that creates double-stranded breaks in DNA sequences. In the amino acid sequence of Cas9, HNH is linearly continuous, whereas RuvC is separated into three regions, one left of the recognition domain, and the other two right of the recognition domain flanking the HNH domain. Cas9 is targeted to a genomic site in a cell by interacting with a guide RNA that hybridizes to a 20-nucleotide DNA sequence that immediately precedes an NGG motif recognized by Cas9. This results in a double-strand break in the genomic DNA of the cell. In some examples, a Cas9 nuclease that requires an NGG protospacer adjacent motif (PAM) immediately 3′ of the region targeted by the guide RNA can be utilized. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to those described in Esvelt et al., Nature Methods 10: 1116-1121 (2013). Various Cas9 nucleases can be utilized in the methods described herein. For example, a Cas9 nuclease that requires an NGG protospacer adjacent motif (PAM) immediately 3′ of the region targeted by the guide RNA, such as SpCas9, can be utilized. Such Cas9 nucleases can be targeted to any region of a genome that contains an NGG sequence. In another example, a Cas9 nuclease that requires an NNGRRT (SEQ ID NO:79) or NNGRR(N) (SEQ ID NO: 80) PAM immediately 3′ of the region targeted by the guide RNA, such as SaCas9, can be utilized. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to those described in Esvelt, K. M., et al., Nature Methods 10(11): 1116-1121 (2013) and those described in Zetsche et al., Cell, Volume 163, Issue 3, p. 759-771, 22 Oct. 2015.

In some cases, the catalytically impaired CRISPR-associated endonuclease is a Cas9 nickase, for example, Cas9 D10A. In some instances, the Cas9 10A in the ABE is encoded by SEQ ID NO: 29. In some instances, the Cas9 10A comprises SEQ ID NO: 30. is Normally, when a Cas9 nickase is bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region. Exemplary Cas9 nickases include Cas9 nucleases having a D10A or H840A mutation.

In some embodiments, the CRISPR-associated endonuclease is a catalytically impaired Cpf1 polypeptide. Cpf1 protein is a Class II, Type V CRISPR/Cas system protein. Cpf1 is a smaller and simpler endonuclease than Cas9 (such as the spCas9). The Cpf1 protein has a RuvC-like endonuclease domain that is similar to the RuvC domain of Cas9 but does not have a HNH endonuclease domain. The N-terminal domain of Cpf1 also does not have the alpha-helical recognition lobe like the Cas9 protein. When cleaving DNA, Cpf1 introduces a sticky-end-like DNA double-stranded break with a 4 or 5 nucleotide overhang. The Cpf1 protein does not need a tracrRNA; rather, the Cpf1 protein functions with only a crRNA. In the context of this disclosure, where the CRISPR-associated endonuclease is a Cpf1 protein, the sgRNA does not comprise a tracr sequence. The sgRNA used with the Cpf1 protein may comprise only a crRNA sequence (constant region). In some examples, a Cpf1 protein that requires an TTTN or TTN PAM (depending on the species, where “N” is an nucleobase) immediately 5′ of the region targeted by the guide RNA can be utilized. Known Cpf1 proteins and derivatives thereof may be used in the context of this disclosure. For example, in some instances, the CRISPR-associated endonuclease is FnCpf1p and the PAM is 5′ TTN, where N is A/C/G or T. In some instances, the CRISPR-associated endonuclease is PaCpf1p and the PAM is 5′ TTTV, where V is A/C or G In certain instances, the CRISPR-associated endonuclease is FnCpf1p and the PAM is 5′ TTN, where N is A/C/G or T, and the PAM is located upstream of the 5′ end of the protospacer. In certain instances, the CRISPR-associated endonuclease is FnCpf1p and the PAM is 5′ CTA and is located upstream of the 5′ end of the protospacer or the target locus. In one example, the CRISPR-associated endonuclease is AsCpf1p and the PAM is 5′ TTTN.

As used herein, “activity” in the context of sgRNA activity, or RNP activity, i.e., RNP activity of a complex comprising: (1) a gRNA and (2) a fusion protein comprising ABE and a catalytically impaired CRISPR-associated endonuclease, refers to the ability of a sgRNA to bind to a target genetic element. Typically, activity also refers to the ability of an ABE RNP (i.e., an sgRNA complexd with an ABE) to edit base pairs, i.e., perform an A to G change in one strand of DNA.

As used herein, the phrase “editing” in the context of editing of a genome of a cell refers to inducing a structural change in the sequence of the genome at a target genomic region, for example, editing performed by an ABE. For example, the editing can take the form of an A to G change in one strand of DNA (or a T to C change on the opposite strand of DNA) at a target genomic region. The nucleotide sequence can encode a polypeptide or a fragment thereof. See, for example, Gaudelli et al., “Programmable base editing of A-T to G-C in genomic DNA without DNA cleavage,” Nature 551: 464-471 (2017).

As used herein, “an adenine base editor” or “ABE” refers to a fusion protein comprising an adenosine deaminase and a catalytically impaired CRISPR-associated endonuclease. In some instances, the adenosine deaminase is a tadA enzyme that deaminates adenine on a single-strand of DNA to form inosine. See, Gaudelli et al, (2017). In some instances, the ABE is a fusion protein comprising a catalytically impaired CRISPR-associated endonuclease and one or more copies, for example, two, three, four copies, etc. of an adenosine deaminase. In some instances the ABE comprises the fusion protein is encoded by a nucleic acid sequence comprising SEQ ID NO: 27. In some instances, the ABE comprises SEQ ID NO: 28.

As used herein, the term “ribonucleoprotein complex,” “RNPs”, and the like refers to a complex between: (1) an ABE and a crRNA (e.g., guide RNA or single guide RNA), (2) an ABE and a trans-activating crRNA (tracrRNA), (3) an ABE, a catalytically impaired CRISPR-associated endonuclease (e.g., Cas9), and a guide RNA, or (4) a combination thereof (e.g., a complex containing the ABE and the catalytically impaired CRISPR-associated endonuclease, a tracrRNA, and a crRNA guide).

As used herein, a “cell” can be any eukaryotic cell, for example, human T cell or a cell capable of differentiating into a T cell, for example, a T cell that expresses a TCR receptor molecule. These include hematopoietic stem cells and cells derived from hematopoietic stem cells. Populations of cells, for example, populations of cells comprising viral particles or genetically modified cells made by any of the genomic editing methods provided herein, are also provided.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin⁻. In some cases, human hematopoietic stem cells are identified as CD34⁺, CD59⁺, Thy1/CD90⁺, CD38^{lo/-}, C-kit/CD117⁺, lin⁻. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, mouse hematopoietic stem cells are identified as CD34^{lo/-}, SCA-1⁺, Thy1^{+/lo}, CD38⁺, C-kit⁺, lin⁻. In some cases, the hematopoietic stem cells are CD150⁺CD48⁻CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes. In some embodiments, the hematopoietic cell is an immune cell, such as a T cell, B cell, macrophage, a natural killer (NK) cell or dendritic cell. In some embodiments the cell is an innate immune cell.

As used herein, the phrase “T cell” refers to a lymphoid cell that expresses a T cell receptor molecule. T cells include human alpha beta (αβ) T cells and human gamma delta (γδ) T cells. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. T cells can be CD4⁺, CD8⁺, or CD4⁺ and CD8⁺. T cells can also be CD4⁻, CD8⁻, or CD4⁻ and CD8⁻. T cells can be helper cells, for example helper cells of type T_{H}1, T_{H}2, T_{H}3, T_{H}9, T_{H}17, or T_{FH}. T cells can be cytotoxic T cells. Regulatory T cells can be FOXP3⁺ or FOXP3⁻. T cells can be alpha/beta T cells or gamma/delta T cells. In some cases, the T cell is a CD4⁺CD25^{hi}CD127^{lo} regulatory T cell. In some cases, the T cell is a regulatory T cell selected from the group consisting of type 1 regulatory (Tr1), T_{H}3, CD8+CD28−, Treg17, and Qa-1 restricted T cells, or a combination or sub-population thereof. In some cases, the T cell is a FOXP3⁺ T cell. In some cases, the T cell is a CD4⁺CD25^{lo}CD127^{hi} effector T cell. In some cases, the T cell is a CD4⁺CD25^{lo}CD127^{hi}CD45RA^{hi}CD45RO⁻ naïve T cell. A T cell can be a recombinant T cell that has been genetically manipulated.

As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Provided herein are compositions, systems, methods of manufacture, and methods for efficient delivery of adenine base editors (ABEs) to eukaryotic cells using viral particles. Using the compositions and methods described herein, ABEs can be efficiently delivered to eukaryotic cells while minimizing sgRNA independent, RNA off-target effects. For example, components, systems, methods of manufacture, and methods for efficient delivery to cells of RNPs comprising (1) an adenosine base pair editor (ABE), wherein the ABE is a fusion protein comprising an adenosine deaminase and a catalytically impaired CRISPR-associated endonuclease; and (2) an sgRNA, via lentivirus-like particles, are provided. The RNPs described herein have a limited half-life, thus reducing the risk of RNA and DNA off-target mediated mutagenesis. Delivery of RNPs into eukaryotic cells allows for efficient delivery, for example, in cells that are difficult to transfect, such as primary cells while reducing off-target effects.

### Mammalian Expression Plasmids

Provided herein are mammalian expression plasmids that are used to deliver CRISPR component coding sequences, i.e., an sgRNA and an ABE, into mammalian cells being used to generate the lentivirus-like particles of this disclosure. For example, provided herein is a mammalian expression plasmid comprising a eukaryotic promoter operably linked to a non-viral nucleic acid sequence, wherein the non-viral nucleic acid sequence comprises; (i) a nucleic acid sequence encoding an adenosine base pair editor (ABE), wherein the ABE is a fusion protein comprising an adenosine deaminase and a catalytically impaired CRISPR-associated endonuclease; and (ii) a guide RNA (gRNA) coding sequence, wherein the gRNA coding sequence comprises at least one aptamer coding sequence.

In the mammalian expression plasmids described herein, one or more copies of an ABE can be fused or linked to a catalytically impaired CRISPR-associate endonuclease. Optionally, the site-directed nuclease is linked to the adenine base editor via a peptide linker. The linker can be between about 2 and about 25 amino acids in length. In some instances, the adenine base editor can be an ABET (for example, ABE7.10 (Gaudelli et al. (2017), ABE 6.3, ABE7.8 or ABE 7.9) or an ABE8 adenine base editor (Gaudelli et al., “Directed evolution of adenine base editors with increased activity and therapeutic application,” Nature Biotechnology 38: 892-900 (2020)).

The mammalian expression plasmids provided herein comprise CRISPR component coding sequences, e.g., the coding sequence for a catalytically impaired CRISPR-associated endonuclease and a gRNA. In some instances, the gRNA coding sequence comprises at least one aptamer coding sequence. In some instances, the at least one aptamer coding sequence may be positioned at the 5′ end or the 3′ end of the gRNA. In some instances, the at least one aptamer coding sequence may be inserted at an internal position within the gRNA such as, for example, at one or more of the loops formed in the folded gRNA. For example, where the gRNA is for the Cas9 protein, the at least one aptamer coding sequence may be positioned at the tetra loop, the stem loop 2 (ST2), or the 3′ end of the gRNA. In some instances, a spacer of 1-30 nucleotides may be positioned between the gRNA the at least one aptamer coding sequence, or flanking the at least one aptamer coding sequence.

In some instances, the mammalian expression vector comprises at least one aptamer coding sequence that encodes an aptamer sequence that is bound specifically by an aptamer-binding protein (ABP). In the context of this disclosure, an aptamer sequence is an RNA sequence that forms a tertiary loop structure that is specifically bound by an ABP. ABPs are RNA-binding proteins or RNA-binding protein domains. Suitable aptamer coding sequences include polynucleotide sequences that encode known bacteriophage aptamer sequences. Exemplary aptamer coding sequences include those encoding the aptamer sequences provided above in Table 1. In some instances, the aptamers are bound by a dimer of ABP. These aptamer sequences are RNA sequences known to be bound specifically by bacteriophage proteins. In some circumstances, the at least one aptamer coding sequence encodes an aptamer sequence bound specifically by an ABP selected from the group consisting of MS2 coat protein, PP7 coat protein, lambda N RNA-binding domain, or Com protein.

TABLE 1
Aptamer-Binding Proteins and Corresponding Aptamer Sequences

| | Aptamer-Binding Proteins | | | |
|---|---|---|---|---|
| | | | lambda N | |
| | MS2 coat | PP7 coat | peptide (amino | Com |
| | protein | protein | acids 1-22) | protein |
| Nucleic Acid | SEQ ID | SEQ ID | SEQ ID | SEQ ID |
| Sequence | NO: 1 | NO: 3 | NO: 5 | NO: 7 |
| Amino Acid | SEQ ID | SEQ ID | SEQ ID | SEQ ID |
| Sequence | NO: 2 | NO: 4 | NO: 6 | NO: 8 |
| Aptamer (RNA) | SEQ ID | SEQ ID | SEQ ID | SEQ ID |
| | NO: 9 | NO: 11 | NO: 13 | NO: 15 |
| | | | (Box-B | |
| | | | aptamer) | |
| Aptamer (DNA) | SEQ ID | SEQ ID | SEQ ID | SEQ ID |
| | NO: 10 | NO: 12 | NO: 14 | NO: 16 |

In some instances, the mammalian expression vector comprises a sgRNA that comprises one aptamer coding sequence downstream thereof. In other instances, the gRNA may comprise 1, 2, 3, 4, 5, 6, 7, 8, 9, 10, 11, or 12 aptamer coding sequences. For example, in some instances, the gRNA may comprise two aptamer coding sequences in tandem.

As used throughout, a sgRNA is a single guide RNA sequence that interacts with a CRISPR-associated endonuclease (a CRISPR site-directed nuclease) and specifically binds to or hybridizes to a target nucleic acid within the genome of a cell (genomic target sequence), such that the sgRNA and the CRISPR-associated endonuclease co-localize to the target nucleic acid in the genome of the cell. Each sgRNA includes a DNA targeting sequence or protospacer sequence of about 10 to 50 nucleotides in length that specifically binds to or hybridizes to a target DNA sequence in the genome. For example, the DNA targeting sequence may be about 11, 12, 13, 14, 15, 16, 17, 18, 19, 20, 21, 22, 23, 24, 25, 26, 27, 28, 29, 30, 31, 32, 33, 34, 36, 37, 38, 39, 40, 41, 42, 43, 44, 45, 46, 47, 48, 49, or 50 nucleotides in length. For example, the DNA targeting sequence may be about 15-30 nucleotides, about 15-25 nucleotides, about 10-25 nucleotides, or about 18-23 nucleotides. In one example, the DNA targeting sequence is about 20 nucleotides. In some embodiments, the sgRNA comprises a crRNA sequence and a transactivating crRNA (tracrRNA) sequence. In some embodiments, the sgRNA does not comprise a tracrRNA sequence.

Generally, the DNA targeting sequence is designed to complement (e.g., perfectly complement) or substantially complement (e.g., having 1-4 mismatches) to the target DNA sequence. In some cases, the DNA targeting sequence can incorporate wobble or degenerate bases to bind multiple genetic elements. In some cases, the 19 nucleotides at the 3′ or 5′ end of the binding region are perfectly complementary to the target genetic element or elements. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is preferably between about 40% and about 60% (e.g., 40%, 45%, 50%, 55%, 60%). In some cases, the binding region, can be selected to begin with a sequence that facilitates efficient transcription of the sgRNA. For example, the binding region can begin at the 5′ end with a G nucleotide. In some cases, the binding region can contain modified nucleotides such as, without limitation, methylated or phosphorylated nucleotides.

As used herein, the term “complementary” or “complementarity” refers to base pairing between nucleotides or nucleic acids, for example, and not to be limiting, base pairing between a sgRNA and a target sequence. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence.

The sgRNA includes a sgRNA constant region that interacts with or binds to the CRISPR-associated endonuclease. In the constructs provided herein, the constant region of an sgRNA can be from about 75 to 250 nucleotides in length. In some examples, the constant region is a modified constant region comprising one, two, three, four, five, six, seven, eight, nine, ten or more nucleotide substitutions in the stem, the stem loop, a hairpin, a region in between hairpins, and/or the nexus of a constant region. In some instances, a modified constant region that has at least 80%, 85%, 90%, or 95% activity, as compared to the activity of the natural or wild-type sgRNA constant region from which the modified constant region is derived, may be used in the constructs described herein. In particular, modifications should not be made at nucleotides that interact directly with a CRISPR-associated endonuclease or at nucleotides that are important for the secondary structure of the constant region.

The mammalian expression plasmids comprise a eukaryotic promoter operably linked to the non-viral nucleic acid sequence. In some instances, a RNA polymerase II promoter is operably linked to the catalytically impaired CRISPR-associated endonuclease coding sequence and a RNA polymerase III promoter is operably linked to the gRNA coding sequence.

The RNA polymerase II promoter sequence is selected from a mammalian species. The RNA polymerase III promoter sequences is selected from a mammalian species. For example, these promoter sequences can be selected from a human, cow, sheep, buffalo, pig, or mouse, to name a few. In some examples, the RNA polymerase II promoter sequence is a CMV, FE1α, or SV40 sequence. In some examples, the RNA polymerase III promoter sequence is a U6 or an H1 sequence. In some examples, the RNA polymerase II sequence is a modified RNA polymerase II sequence. For example, the RNA polymerase II sequences having at least 80%, 85%, 90%, 95%, or 99% identity to a wild-type RNA polymerase II promoter sequence from any mammalian species can be used in the constructs provided herein. In some examples, the RNA polymerase III sequence is a modified RNA polymerase III sequence. For example, the RNA polymerase III sequences having at least 80%, 85%, 90%, 95%, or 99% identity to a wild-type RNA polymerase III promoter sequence from any mammalian species can be used in the constructs provided herein. Those of skill in the art readily understand how to determine the identity of two polypeptides or nucleic acids. For example, the identity can be calculated after aligning the two sequences so that the identity is at its highest level. Another way of calculating identity can be performed by published algorithms. For example, optimal alignment of sequences for comparison can be conducted using the algorithm of Needleman and Wunsch, J. Mol. Biol. 48(3): 443-453 (1970). In some instances, the eukaryotic promoter is an inducible or regulatable promoter.

Coding sequences transcribed from a RNA pol II promoter include a poly(A) signal and a transcription terminator sequence downstream of the coding sequence. Commonly used mammalian terminators (SV40, hGH, BGH, and rbGlob) include the sequence motif AAUAAA (SEQ ID NO: 81) which promotes both polyadenylation and termination. Coding sequences transcribed from a RNA pol III promoter include a simple run of T residues downstream of the coding sequence as a terminator sequence. The role of the terminator, a sequence-based element, is to define the end of a transcriptional unit (such as a gene) and initiate the process of releasing the newly synthesized RNA from the transcription machinery. Terminators are found downstream of the gene to be transcribed, and typically occur directly after any 3′ regulatory elements, such as the polyadenylation or poly(A) signal.

In some instances, the mammalian expression plasmid may also include at least one polynucleotide sequence encoding a RNA-stabilizing sequence positioned downstream of the CRISPR component coding sequence or the aptamer coding sequence if positioned downstream of the CRISPR component coding sequence. The polynucleotide sequence encoding the RNA-stabilizing sequence is transcribed downstream of the CRISPR/Cas system component coding sequence and stabilizes the longevity of the transcribed RNA sequence. In one example, the polynucleotide sequence encoding the RNA-stabilizing sequence is positioned downstream of the catalytically impaired CRISPR-associated endonuclease coding sequence. In another example, the polynucleotide sequence encoding the RNA-stabilizing sequence is positioned downstream of the gRNA coding sequence. An exemplary RNA-stabilizing sequence is the sequence of the 3′ UTR of human beta globin gene as set forth in SEQ ID NO:17 (DNA) and SEQ ID NO:18 (RNA). Another example of an RNA-stabilizing sequence is SEQ ID NO: 34 which comprises two copies of SEQ ID NO: 17. Other RNA-stabilizing sequences are described in Hayashi, T. et al., Developmental Dynamics 239(7):2034-2040 (2010) and Newbury, S. et al., Cell 48(2):297-310 (1987). In some instances, a spacer of 1-30 nucleotides may be positioned between the CRISPR component coding sequence and the at least one polynucleotide sequence encoding RNA-stabilizing sequence.

In some instances, the mammalian expression plasmid may comprise one or more expression cassettes. In some instances the mammalian expression plasmid comprises a first expression cassette that encodes the ABE and a second expression cassette that encodes the gRNA comprising at least one aptamer. In some instances, the mammalian expression plasmid may also comprise a reporter gene.

### Systems

Another aspect of this disclosure are lentiviral packaging systems. Such systems include the mammalian expression plasmids described in this disclosure. These systems are useful in providing components for introduction into mammalian cells to generate the lentivirus-like particles described in this disclosure.

In some instances, the system includes a lentiviral packaging plasmid comprising a eukaryotic promoter operably linked to a viral sequence, for example, a Gag nucleotide sequence, wherein the Gag nucleotide sequence comprises a nucleocapsid (NC) coding sequence and a matrix protein (MA) coding sequence, wherein one or both of the NC coding sequence or the MA coding sequence comprise at least one non-viral aptamer-binding protein (ABP) nucleotide sequence, and wherein the packaging plasmid does not encode a functional integrase protein.

For example, provided herein is a lentiviral packaging system comprising: (a) a packaging plasmid comprising a eukaryotic promoter operably linked to a Gag nucleotide sequence, wherein the Gag nucleotide sequence comprises a nucleocapsid (NC) coding sequence and a matrix protein (MA) coding sequence, wherein one or both of the NC coding sequence or the MA coding sequence comprises at least one non-viral aptamer-binding protein (ABP) nucleotide sequence, and wherein the packaging plasmid does not encode a functional integrase protein; (b) at least one mammalian expression plasmid comprising (i) a nucleic acid sequence encoding an adenosine base pair editor (ABE), wherein the ABE is a fusion protein comprising an adenosine deaminase and a catalytically impaired CRISPR-associated endonuclease and (ii) a gRNA described herein; and (c) an envelope plasmid comprising an envelope glycoprotein coding sequence.

The system may include a second generation packaging plasmid or third generation packaging plasmids or modified versions thereof. In some instances, the packaging plasmid includes the Gag nucleotide sequence as described above and further comprises a Rev nucleotide sequence and a Tat nucleotide sequence. In other instances, the system includes a first packaging plasmid including a Gag nucleotide sequence as described above and a second packaging plasmid comprising a Rev nucleotide sequence. In each of the packaging plasmids, the viral protein coding sequences are operably linked to a eukaryotic promoter for example, each individually or one promoter for multiple protein coding sequences. The system may include a second generation packaging plasmid or third generation packaging plasmids or modified versions thereof.

In some instances, the ABP coding sequence is at the 5′ end or 3′ end of the viral protein coding sequence, i.e., at the 5′ end or the 3′ end of the NC or MA coding sequence. In some instances, the ABP coding sequence may be inserted into the viral protein coding sequence such that the encoded ABP is fused to the viral protein. The ABP coding sequence may be inserted in frame at an internal position within the viral protein coding sequence. When positioned in frame at an internal position near the 5′ or 3′ end of the viral protein coding sequence, the ABP coding sequence is positioned so as not to disrupt processing sequences such as those described in Tritch, R. J. et al., J. Virol. 65(2):922-30 (1991) and Scarlata, S. and Carter, C., Biochimica et Biophysica Acta—Biomembranes 1614(1):62-72 (2003), which are incorporated herein by reference in their entirety. For example, the Gag nucleotide sequence encodes, inter alia, the NC coding sequence and the MA coding sequence, and the Gag precursor protein is processed by proteolytic cleavage into separate mature viral proteins. The in frame insertion of the ABP coding sequence would not disrupt the nucleotides encoding the processing sequences for proteolytic cleavage. In some instances, nucleotides in the viral protein coding sequence may be replaced with the ABP protein coding sequence. In some instances, a linker sequence encoding 3-6 amino acids may be positioned between the viral protein coding sequence and the ABP coding sequence, or flanking the ABP coding sequence, to help facilitate proper folding of the protein domains upon expression.

In one example, the modified viral protein is NC and the ABP coding sequence is inserted at the 5′ end or the 3′ end of the NC coding sequence. In another example, the modified viral protein is NC and the ABP coding sequence is inserted before or after one of the zinc finger (ZF) domains. For example, the ABP coding sequence may be inserted after the last codon of the second ZF (ZF2) domain. In another example, the ABP coding sequence may be inserted before the first codon of the ZF2 domain. In another example, the ABP coding sequence may be inserted before the first codon of the first ZF (ZF1) domain. In another example, the ABP coding sequence may be inserted after the last codon of the first ZF (ZF1) domain. In some instances, the ABP coding sequence is inserted into the NC coding sequence in a manner that does not disrupt the highly positive stretch of amino acids in the NC protein.

In another example, the modified viral protein is MA and the ABP coding sequence is inserted at the 5′ end or the 3′ end of the MA coding sequence. In another example, the ABP coding sequence is inserted in frame at an internal position within the MA coding sequence. In some instances, nucleotides in the MA coding sequence may be replaced with the ABP protein coding sequence. For example, the nucleotides encoding amino acids 44-132 of the MA protein may be replaced with the ABP coding sequence. In another example, the ABP coding sequence is inserted prior to the codon encoding amino acid 44 of the MA protein. In another example, the ABP coding sequence is inserted after the codon encoding amino acid 132 of the MA protein.

In some instances, the system includes a packaging plasmid comprising a eukaryotic promoter operably linked to a NEF coding sequence or a VPR coding sequence, wherein the NEF coding sequence or the VPR coding sequence comprises at least one non-viral ABP nucleotide sequence. The system may include a second generation packaging plasmid or third generation packaging plasmids or modified versions thereof. In some instances, the packaging plasmid includes a Gag nucleotide sequence, a Rev nucleotide sequence, and a Tat nucleotide sequence. In other instances, the system includes a first packaging plasmid including a Gag nucleotide sequence and a second packaging plasmid comprising a Rev nucleotide sequence.

In some instances, the modified viral protein is VPR and the ABP coding sequence is inserted at the 5′ end or the 3′ end of the VPR coding sequence. In one example, the ABP coding sequence is inserted at the 5′ end of the VPR coding sequence.

In other instances, the modified viral protein is NEF and the ABP coding sequence is inserted at the 5′ end or the 3′ end of the NEF coding sequence. In one example, the ABP coding sequence is inserted at the 3′ end of the NEF coding sequence.

In some instances, the coding sequence of the viral protein may be one of SEQ ID NO:19, SEQ ID NO:20, SEQ ID NO:21, or SEQ ID NO:25. In some instances, the amino acid sequence of the viral protein may be one of SEQ ID NO:20, SEQ ID NO:22, SEQ ID NO:24, or SEQ ID NO:26. In some instances, the lentiviral packaging plasmid comprises a sequence encoding at least one of SEQ ID NO:20, SEQ ID NO:22, SEQ ID NO:24, or SEQ ID NO:26 operably linked to a eukaryotic promoter. In some instances, if the viral protein is NEF, the polypeptide may comprise three mutations that enhances packaging in the viral capsid such as, for example, the following substitution mutations: G3C, V153L, and E177G.

In some instances, the plasmids may encode one or more viral proteins that comprise two or more aptamer-binding proteins fused thereto. In certain instances, the Gag nucleotide sequence of the lentiviral packaging plasmid may comprise a NC coding sequence and a MA coding sequence and where one or both of the NC coding sequence or the MA coding sequence comprises a first non-viral ABP nucleotide sequence and a second non-viral ABP nucleotide sequence. The first non-viral ABP nucleotide sequence and the second non-viral ABP nucleotide sequence may both encode the same ABP. Alternatively, the first non-viral ABP nucleotide sequence and the second non-viral ABP nucleotide sequence encode different ABPs. In some instances, the Gag nucleotide sequence of the lentiviral packaging plasmid may comprise a NC coding sequence comprising at least one first non-viral ABP nucleotide sequence and a MA coding sequence comprising at least one second non-viral ABP nucleotide sequence. The at least one first non-viral ABP nucleotide sequence and the at least one second non-viral ABP nucleotide sequence may both encode the same ABP. Alternatively, the at least one first non-viral ABP nucleotide sequence and the at least one second non-viral ABP nucleotide sequence encode different ABPs.

In certain instances, the packaging plasmid may encode a VPR coding sequence or a NEF coding sequence and where the VPR coding sequence or the NEF coding sequence comprises a first non-viral ABP nucleotide sequence and a second non-viral ABP nucleotide sequence. The first non-viral ABP nucleotide sequence and the second non-viral ABP nucleotide sequence may both encode the same ABP. Alternatively, the first non-viral ABP nucleotide sequence and the second non-viral ABP nucleotide sequence encode different ABPs.

A non-viral aptamer-binding protein (ABP) nucleotide sequence encodes a polypeptide sequence that binds to an RNA aptamer sequence. Several non-viral ABPs are suitable for use in this disclosure. In particular, suitable ABPs include bacteriophage RNA-binding proteins that bind specifically to RNA sequences that form stem-loop structures referred to as RNA aptamer sequences. Exemplary non-viral aptamer binding protein include MS2 coat protein, PP7 coat protein, lambda N peptide, and Com (control of mom) protein. The lambda N peptide may be amino acids 1-22 of the lambda N protein, which are the RNA-binding domain of the protein. In some instances, the ABPs bind to their aptamers as dimers. Information about these ABP and the aptamer sequences to which they bind is provided in Table 1. In some embodiments, the at least one non-viral ABP nucleotide sequence encodes a polypeptide having the sequence set forth in any of SEQ ID NO:10, SEQ ID NO:12, SEQ ID NO:14, or SEQ ID NO:16. In some embodiments, the at least one non-viral ABP nucleotide sequence comprises any of SEQ ID NO:9, SEQ ID NO:11, SEQ ID NO:13, or SEQ ID NO:15.

A feature of the lentiviral packaging plasmids provided herein is that they may not encode a functional integrase protein. When the packaging plasmids do not encode a functional integrase protein and they are used in the systems and methods described herein, there is substantially reduced risk the nucleic acid molecules carried by the lentivirus-like particles produced using these packaging plasmids will integrate into the genome of the transduced eukaryotic cell. In some instances, the lentiviral packaging plasmid comprises an integrase coding sequence with an integrase-inactivating mutation therein. For example, the integrase-inactivating mutation may be an aspartic acid to valine mutation at amino acid position 64 (D64V) of the integrase protein encoded by the integrase coding sequence. In some instances, the lentiviral packaging plasmid comprises a deletion of all or a portion of an integrase coding sequence.

In some embodiments, the lentiviral packaging plasmids comprise a eukaryotic promoter operably linked to the Gag nucleotide sequence. In some embodiments, the mammalian expression plasmids comprise a eukaryotic promoter operably linked to the VPR coding sequence or the NEF coding sequence. In some instances, the eukaryotic promoter is a RNA polymerase II promoter. The RNA polymerase II promoter sequence is selected from a mammalian species. For example, the promoter sequence can be selected from a human, cow, sheep, buffalo, pig, or mouse, to name a few. In some examples, the RNA polymerase II promoter sequence is a CMV, FE1α, or SV40 sequence. In some examples, the RNA polymerase II sequence is a modified RNA polymerase II sequence. For example, the RNA polymerase II sequences having at least 80%, 85%, 90%, 95%, or 99% identity to a wild-type RNA polymerase II promoter sequence from any mammalian species can be used in the constructs provided herein. Those of skill in the art readily understand how to determine the identity of two polypeptides or nucleic acids. For example, the identity can be calculated after aligning the two sequences so that the identity is at its highest level. Another way of calculating identity can be performed by published algorithms. For example, optimal alignment of sequences for comparison can be conducted using the algorithm of Needleman and Wunsch, J. Mol. Biol. 48: 443 (1970). In some instances, the eukaryotic promoter is an inducible promoter.

Coding sequences transcribed from a RNA pol II promoter include a poly(A) signal and a transcription terminator sequence downstream of the coding sequence. Commonly used mammalian terminators (e.g., SV40, hGH, BGH, and rbGlob) include the sequence motif AAUAAA which promotes both polyadenylation and termination. The role of the terminator, a sequence-based element, is to define the end of a transcriptional unit (such as a gene) and initiate the process of releasing the newly synthesized RNA from the transcription machinery. Terminators are found downstream of the gene to be transcribed, and typically occur directly after any 3′ regulatory elements, such as the polyadenylation or poly(A) signal.

In some instances, the lentiviral packaging plasmids may comprise one or more expression cassettes.

The system also can include an envelope plasmid having an envelope coding sequence that encodes a viral envelope glycoprotein. For example, the Env nucleotide sequence may encode VSV-G. The envelope coding sequence is operably linked to a eukaryotic promoter. Appropriate eukaryotic promoters are described above. In some instances, the eukaryotic promoter is a RNA pol II promoter.

The system can comprise any of the packaging plasmids, envelope plasmids and mammalian expression plasmids, i.e., a mammalian expresson plasmid comprising (i) a nucleic acid sequence encoding an ABE; and (ii) a gRNA comprising at least one aptamer, described herein. When any of the packaging plasmids, mammalian expression plasmids and envelope plasmids described herein are delivered to eukaryotic cells as a system, the gRNA expressed by the mammalian expression plasmid forms a complex with the catalytically-impaired CRISPR-associated endonuclease expressed by the mammalian expression plasmids to form an RNP that is packaged by the viral particles produced by the eukaryotic cells, via the interaction between the aptamer fused or linked to the gRNA and the ABP linked to the viral protein expressed by the packaging plasmid.

Also provided herein are kits the include the components of the systems described in this disclosure. In some embodiments, the kits include one or more of the plasmids described herein.

### Lentivirus-Like Particles

In another aspect, provided are lentivirus-like particles, for example, lentivirus-like particles made by any of the methods described herein. As used herein, a lentivirus-like particle is multiprotein structure that mimics the organization and conformation of authentic native viruses but lacks the viral genome. A plurality of lentivirus-like particles are also provided. The lentivirus-like particles contain a modified lentiviral protein that is a fusion protein in which at least one aptamer-binding protein is fused to one or more viral proteins. In the context of this disclosure, the modified viral protein may be structural or non-structural. Exemplary structural proteins are lentiviral nucleocapsid (NC) protein and matrix (MA) protein. Exemplary non-structural proteins are viral protein R (VPR) and negative regulatory factor (NEF). In some instances, the particles contain a fusion protein comprising a NC protein and a MA protein where one or both thereof are fused with at least one non-viral aptamer binding protein (ABP). The NC protein of the particles may have two functional zinc finger protein domains. In particular, retention of the second NC zinc finger domain may preserve the efficiency of viral assembly and budding. In some instances, the particles contain a fusion protein comprising a VPR protein or a NEF protein where the VPR protein or the NEF protein are fused with at least one non-viral ABP. The particles also contain an RNP comprising: (i) an adenosine base pair editor (ABE), wherein the ABE is a fusion protein comprising an adenosine deaminase and a catalytically impaired CRISPR-associated endonuclease; and (ii) a gRNA. Any of the mammalian expression plasmids described herein comprising a non-viral nucleic acid sequence, wherein at least one aptamer is attached or inserted into the gRNA sequence, can be used to generated lentivirus-like particles containing RNPs. In some instances, the lentivirus-like particles do not contain a functional integrase protein. These virus-like particles are useful to transduce eukaryotic cells of interest.

The particles may comprise a viral fusion protein comprising one or more ABPs. In some instances, the particles contain a NC protein, a MA protein, or both, where one or both of the NC protein or MA protein are fused with one or more non-viral ABP. In some instances, lentivirus-like particles comprise a NC protein fused with at least one non-viral ABP. In some instances, lentivirus-like particles comprise a MA protein fused with at least one non-viral ABP. In some instances, the lentivirus-like particles may comprise a NC protein and a MA protein, where one or both of the NC protein or the MA protein may be fused with two non-viral ABP proteins, a first non-viral ABP and a second non-viral ABP fused to a C′ terminal end of the first non-viral ABP (i.e. in tandem). In certain instances, the particles may contain one or both of a NC protein or a MA protein fused with a first non-viral ABP and a second non-viral ABP.

In some instances, the lentivirus-like particle contains a VPR protein or a NEF protein, where the VPR protein or the NEF protein is fused to one or more non-viral ABP. In some instances, the lentivirus-like particle contains a VPR protein or a NEF protein fused to two non-viral ABP, a first non-viral ABP and a second non-viral ABP fused to a C′ terminal end of the first non-viral ABP (i.e. in tandem). In some instances, the lentivirus-like particle contains a VPR protein or a NEF protein fused to a first non-viral ABP and a second non-viral ABP. The first non-viral ABP and the second non-viral ABP may both be the same ABP. Alternatively, the first non-viral ABP and the second non-viral ABP may be different ABPs. In some instances, the lentivirus-like particles may comprise a NC protein with at least one first non-viral ABP fused to MA protein with at least one second non-viral ABP fused to its C′ terminal end. The at least one first non-viral ABP and the at least one second non-viral ABP both be the same ABP. Alternatively, the at least one first non-viral ABP protein and the at least one second non-viral ABP may be different ABPs. The first non-viral ABP and the second non-viral ABP may both be the same ABP. Alternatively, the first non-viral ABP and the second non-viral ABP may be different ABPs.

A non-viral ABP is a polypeptide sequence that binds to an RNA aptamer sequence. Several non-viral ABPs are suitable for use in this disclosure. In particular, suitable ABPs include bacteriophage RNA-binding proteins that bind specifically to known RNA aptamer sequences, which are RNA sequences that form stem-loop structures. Exemplary non-viral aptamer binding protein include MS2 coat protein, PP7 coat protein, lambda N peptide, and Com (Control of mom) protein. The lambda N peptide may be amino acids 1-22 of the lambda N protein, which are the RNA-binding domain of the protein. Information about these ABP and the aptamer sequences to which they bind is provided above in Table 1.

The lentivirus-like particles may comprise various lentiviral proteins. However, in some instances, the lentivirus-like particles do not comprise all of the types of proteins or nucleic acids found in native lentiviruses. In some instances, the particles may contain NC, MA, CA, SP1, SP2, P6, POL, ENV, TAT, REV, VIF, VPU, VPR, and/or NEF proteins, or a derivative, combination, or portion of any thereof. In some instances, the particles may contain NC, MA, CA, SP1, SP2, P6, and POL. In some instances, the lentivirus-like particles may comprise only those proteins that form the viral shell (capsid). In some instances, one or more lentiviral proteins may be excluded in full or in part from the lentivirus-like particles. For example, in some instances, the lentivirus-like particles may not contain a POL protein or may comprise a non-functional version of a POL protein such as, for example, a POL protein with an inactivating point mutation or an inactivating truncation. In another example, the lentivirus-like particles may not contain an integrase protein or may comprise a non-functional version of an integrase protein such as, for example, an integrase protein with an inactivating point mutation or an inactivating truncation. For example, the lentivirus-like particle may contain a non-functional integrase protein comprising an aspartic acid to valine mutation at amino acid position 64 (D64V). In another example, the lentivirus-like particles may not contain a reverse transcriptase protein or may comprise a non-functional version of a reverse transcriptase protein such as, for example, a reverse transcriptase protein with an inactivating point mutation or an inactivating truncation.

As set forth above, gRNA generally comprises a DNA targeting sequence and a constant region that interacts with the CRISPR-associated endonuclease. In some instances, the gRNA may comprise a transactivating crRNA (tracrRNA) sequence. For example, the gRNA may comprise a tracrRNA where it is to be used in conjunction with a Cas9 protein or derivative. In other instances, the gRNA does not comprise a tracrRNA sequence. For example, the gRNA may not comprise a tracrRNA sequence where it is to be used in conjunction with a Cpf1 protein or derivative.

In some instances, the gRNA comprises at least one aptamer sequence. In some instances, the at least one aptamer sequence may be positioned at the 5′ end or the 3′ end of the gRNA. In some instances, the at least one aptamer sequence may be inserted at an internal position within the gRNA such as, for example, at one or more of the loops formed in the folded gRNA. For example, where the gRNA is for a Cas9 protein, the at least one aptamer sequence may be positioned at the tetra loop, the stem loop 2 (ST2), or the 3′ end of the gRNA. In some instances, a spacer of 1-30 ribonucleotides may be positioned between the gRNA and the at least one aptamer sequence, or flanking the at least one aptamer sequence. In certain instances, at least one aptamer sequence does not interfere with lentivirus-like particle transduction of eukaryotic cells. For example, at least one non-viral ABP fused to one or more of the NC protein, the MA protein, the VPR protein, or the NEF protein may not interfere with lentivirus-like particle transduction of eukaryotic cells.

### Gene Editing Methods

Described herein are methods of using the plasmids and systems provided in this disclosure in CRISPR/Cas systems for editing DNA targets, for example, a gene, in the genome of a eukaryotic cell.

In the methods provided herein, eukaryotic cells comprising a target genomic sequence of interest to be modified are transduced with lentivirus-like particles that contain a viral fusion protein comprising a viral protein fused to at least one aptamer-binding protein (ABP) and an RNP comprising (1) a gRNA and (2) an adenosine base pair editor (ABE), wherein the ABE is a fusion protein comprising an adenosine deaminase and a catalytically impaired CRISPR-associated endonuclease.

An advantage of the provided methods is reduced guide independent RNA off-target gene editing events associated with ABEs. For example, in the methods provided herein, guide-independent RNA off-target activity can be reduced by at least 5%, 10%, 20%, 30%, 40%, 50%, 60%, 70%, 90%, 95%, 99% or greater, as compared to RNA off-target activity when RNPs are delivered using non-lentiviral delivery. In some instances, guide independent DNA off-target gene editing events are also reduced. For example, in the methods provided herein, guide-dependent DNA off-target activity can be reduced by at least 5%, 10%, 20%, 30%, 40%, 50%, 60%, 70%, 90%, 95%, 99% or greater when RNPs are delivered using non-lentiviral delivery. Also, when lentivirus-like particles lacking integrase activity are used in the method, there is reduced risk of integration into the cell genome of any of the nucleic acids carried by the particles. In some instances, the lentiviral-particles used lack portions of the lentiviral genomic sequences that are essential for viral replication and, as such, reduce the risk of continued particle production. Another advantage of the provided components is that the viral fusion protein may increase packaging of RNPs, into the lentivirus-like particles, which in turn increase genome editing efficiency.

In some instances, the transduced eukaryotic cells are mammalian cells. In some instances, the eukaryotic cells may be in vitro cultured cells. In some instances, the eukaryotic cells may be ex vivo cells obtained from a subject. In other instances, the eukaryotic cells are present in a subject. As used throughout, by subject is meant an individual. For example, the subject is a mammal, such as a primate, and, more specifically, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations are contemplated herein. The term does not denote a particular age or sex. Thus, adult and newborn subjects, whether male or female, are intended to be covered. As used herein, patient or subject may be used interchangeably and can refer to a subject afflicted with a disease or disorder. The lentivirus-like particles provided herein may be administered to the subject, for example, injected into a subject, according to known, routine methods. Exemplary modes of administration include oral, rectal, transmucosal, topical, intranasal, inhalation (e.g., via an aerosol), buccal (e.g., sublingual), vaginal, intrathecal, intraocular, transdermal, intradermal, intrapleural, intracerebral, and intraarticular), topical, and the like, as well as direct tissue or organ injection. Administration can also be to a tumor. The most suitable route in any given case will depend on the nature and severity of the condition being treated and on the nature of the particular lentivirus-like particle that is being used. In some instances, the lentivirus-like particles are injected intravenously (IV), intraperitoneally (IP), intramuscularly, or into a specific organ or tissue. In some embodiments, more than one administration (e.g., two, three, four or more administrations) may be employed to achieve the desired level of gene editing over a period of various intervals, e.g., daily, weekly, monthly, yearly, etc.

An effective amount of any of the recombinant lentivirus-like particles described herein will vary and can be determined by one of skill in the art through experimentation and/or clinical trials. For example, an effective dose can be from about 10⁶ to about 10¹⁵ lentivirus-like particles, for example, from about 10⁶ to about 10¹⁴, from about 10⁶ to about 10¹³, from about 10⁶ to about 10¹² lentivirus-like particles, from about 10⁶ to about 10¹², from about 10⁶ to about 10¹¹, or from about 10⁶ to about 10¹¹ lentivirus-like particles. Other effective dosages can be readily established by one of ordinary skill in the art through routine trials establishing dose response curves. See, for example, Mangeot et al. “Genome editing in primary cells and in vivo using viral-derived Nanoblades loaded with Cas9-sgRNA ribonucleoproteins,” Nat Commun 10, 45 (2019). https://doi.org/10.1038/s41467-018-07845-z.

In some instances, the provided methods are for modifying a target locus of interest, the method comprising transducing a plurality of eukaryotic cells with a plurality of viral particles, wherein the plurality of viral particles comprise (i) a fusion protein comprising a viral protein, for example, NC, MA, VRP, or NEF, wherein the viral protein comprises at least one non-viral aptamer binding protein (ABP); and (ii) a ribonucleotide protein (RNP) complex comprising (1) a gRNA and (2) an ABE, wherein the RNP is capable of binding (e.g., preferentially binding) via the gRNA, to the genomic target sequence in genomic DNA of the cell and the ABE alters the genomic DNA of the cell. As described above, the RNPs are packaged into the viral particles via the interaction of an aptamer sequence attached to or inserted into a gRNA sequence that forms a complex with the catalytically impaired CRISPR-associated endonuclease.

The methods described can be used with any catalytically impaired CRISPR-associated endonuclease that requires a constant region of an sgRNA for function. These include, but are not limited to RNA-guided site-directed nucleases. Examples include nucleases present in any bacterial species that encodes a Type II or V CRISPR/Cas system. Suitable CRISPR-associated endonucleases are described throughout this disclosure. For example, and not to be limiting, the site-directed nuclease can be a catalytically impaired Cas9 polypeptide, a catalytically impaired Cpf1 polypeptide, a catalytically impaired Cas9 nickase, or derivatives of any thereof.

Generally, the sgRNA is targeted to specific regions at or near a gene. In some instances, the sgRNA can be targeted to a region where single base changes are necessary, for example, to correct a single base mutation in the human beta-globin gene that causes sickle cell anemia. The sgRNA allows the RNPs described herein to a specific site in the genomic sequence of a cell. Once the RNP binds to the specific site in the genomic sequence, the adenine base editor, catalyzes adenosine (A) to inosine formation in one strand, while the catalytically impaired endonuclease, for example, Cas9 D10A nicks the opposite strand, i.e., the non-edited strand. Since inosine is read as guanosine by polymerase enzymes, DNA repair and replication mechanisms replace the original A-T base pair with a G-C base pair at the target site. See, Gaudelli et al. (2017).

In some instances, the modifications to the system components as described in this disclosure do not impair how the system components function following transduction into eukaryotic cells. Rather, the components may function similarly or better than unmodified components upon transduction into eukaryotic cells. For example, the viral fusion proteins in the lentivirus-like particles may not interfere with the lentivirus-like particle transduction of eukaryotic cells. Similarly, if the RNPs packaged in the lentivirus-like particles comprise at least one aptamer sequence, the at least one aptamer sequence may not interfere with the lentivirus-like particle transduction of eukaryotic cells. In some instances, the lentivirus-like proteins containing viral fusion protein may result in greater gene editing upon transduction into eukaryotic cells relative to lentivirus-like particles that do not comprise a viral fusion protein. In one example the viral fusion protein may be a NC-ABP fusion protein, such as a NC-MS2 fusion protein or NC-PP7 fusion protein. In one example, the NC fusion protein is fused to one or two ABPs, such as one or two MS2 proteins, one or two PP7 proteins, or one MS2 protein and one PP7 protein.

The eukaryotic cells can be in vitro, ex vivo or in vivo. In some embodiments, the cell is a primary cell (isolated from a subject). As used herein, a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. In some embodiments, the cells are cultured under conditions effective for expanding the population of modified cells. In some embodiments, cells modified by any of the methods provided herein are purified. In some cases, cells are removed from a subject, modified using any of the methods described herein and re-administered to the patient.

In some instances, once the cells have been transduced with the viral particles described above, the cells are cultured for a sufficient amount of time to allow for gene editing to occur, such that a pool of cells expressing a detectable phenotype can be selected from the plurality of transduced cells. The phenotype can be, for example, cell growth, survival, or proliferation. In some examples, the phenotype is cell growth, survival, or proliferation in the presence of an agent, such as a cytotoxic agent, an oncogene, a tumor suppressor, a transcription factor, a kinase (e.g., a receptor tyrosine kinase), a gene (e.g., an exogenous gene) under the control of a promoter (e.g., a heterologous promoter), a checkpoint gene or cell cycle regulator, a growth factor, a hormone, a DNA damaging agent, a drug, or a chemotherapeutic. The phenotype can also be protein expression, RNA expression, protein activity, or cell motility, migration, or invasiveness. In some examples, the selecting the cells on the basis of the phenotype comprises fluorescence activated cell sorting, affinity purification of cells, or selection based on cell motility.

In some examples, the selecting the cells comprises analysis of the genomic DNA of the cells such as by amplification, sequencing, SNP analysis, etc. Sequencing methods include, but are not limited to, shotgun sequencing, bridge PCR, Sanger sequencing (including microfluidic Sanger sequencing), pyrosequencing, massively parallel signature sequencing, nanopore DNA sequencing, single molecule real-time sequencing (SMRT) (Pacific Biosciences, Menlo Park, CA), ion semiconductor sequencing, ligation sequencing, sequencing by synthesis (Illumina, San Diego, Ca), Polony sequencing, 454 sequencing, solid phase sequencing, DNA nanoball sequencing, heliscope single molecule sequencing, mass spectroscopy sequencing, pyrosequencing, Supported Oligo Ligation Detection (SOLiD) sequencing, DNA microarray sequencing, RNAP sequencing, tunneling currents DNA sequencing, and any other DNA sequencing method identified in the future. One or more of the sequencing methods described herein can be used in high throughput sequencing methods. As used herein, the term “high throughput sequencing” refers to all methods related to sequencing nucleic acids where more than one nucleic acid sequence is sequenced at a given time.

### Methods of Treatment

Any of the methods and compositions described herein can be used to treat a disease (e.g., cancer, a blood disorder (for example, sickle cell anemia or beta thalassemia), an infectious disease, an autoimmune disease, transplantation rejection, graft vs. host disease or other inflammatory disorder) in a subject.

In some methods, the cancer to be treated is selected from a cancer of B-cell origin, breast cancer, gastric cancer, neuroblastoma, osteosarcoma, lung cancer, colon cancer, chronic myeloid cancer, leukemia (e.g., acute myeloid leukemia, chronic lymphocytic leukemia (CLL) or acute lymphocytic leukemia (ALL)), prostate cancer, colon cancer, renal cell carcinoma, liver cancer, kidney cancer, ovarian cancer, stomach cancer, testicular cancer, rhabdomyosarcoma, and Hodgkin's lymphoma. In some embodiments, the cancer of B-cell origin is selected from the group consisting of B-lineage acute lymphoblastic leukemia, B-cell chronic lymphocytic leukemia, and B-cell non-Hodgkin's lymphoma

In some methods, the cells of the subject are modified in vivo. In some methods, the method of treating a disease in a subject comprises: a) obtaining cells from the subject; b) modifying the cells using any of the methods provided herein; and c) administering the modified cells to the subject. See, for example, Milone and O'Doherty “Clinical sue of lentiviral vectors,” Leukemia 32, 1529-1541 (2018). Optionally, the disease is selected from the group consisting of cancer, a blood disorder (for example, sickle cell anemia or beta thalassemia), an infectious disease, an autoimmune disease, transplantation rejection, graft vs. host disease or other inflammatory disorder in a subject. In some methods for treating cancer, the cells obtained from the subject are modified to express a tumor specific antigen. As used throughout, the phrase “tumor-specific antigen” means an antigen that is unique to cancer cells or is expressed more abundantly in cancer cells than in in non-cancerous cells. Optionally, the cells obtained from the subject are T cells. Optionally, the modified cells are expanded prior to administration to the subject.

The lentivirus-like particles or cells described herein can be formulated as a pharmaceutical composition. Therefore, provided herein is a pharmaceutical composition comprising any of the lentivirus-like particles described herein. Also provided is a pharmaceutical composition comprising any of the modified cells described herein Optionally, the pharmaceutical composition can further comprise a carrier. The term carrier means a compound, composition, substance, or structure that, when in combination with lentivirus-like particles or cells, aids or facilitates preparation, storage, administration, delivery, effectiveness, selectivity, or any other feature of the lentivirus-like particles or cells for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

Different arrangements of the components depicted in the drawings or described above, as well as components and steps not shown or described are possible. Similarly, some features and sub-combinations are useful and may be employed without reference to other features and sub-combinations. Embodiments of the disclosure have been described for illustrative and not restrictive purposes, and alternative embodiments will become apparent to readers of this patent. Accordingly, the present disclosure is not limited to the embodiments described above or depicted in the drawings, and various embodiments and modifications can be made without departing from the scope of the claims below.

Publications cited herein and the material for which they are cited are hereby specifically incorporated by reference in their entireties.

### Examples

### Plasmids

pMD2.G (Addgene #12259), pCMV_ABEmax (Addgene #112095) (Koblan et al. Nat Biotechnol 2018, 36(9): 843-846). and psPAX2-D64V (Addgene #63586) (Certo et al. Nat Methods 2011, 8(8): 671-6). were purchased from Addgene. The plasmid for expressing ABE7.10 in E. coli has been described earlier (Kim et al., Nat Biotechnol 2019, 37 (4), 430-435). Other plasmids were generated, as shown in Table 2. Gene synthesis was done by GenScript Inc. All constructs generated were confirmed by Sanger sequencing. Sequence information for primers and oligonucleotides are listed in Table 3. ABE target sequences and the oligos used for making the sgRNA expression constructs are listed in Table 4. It is understood that the sequences for the components of the plasmids listed in Table 2 can be separated by nucleic acid linkers, for example, linkers of about 2 to 100 bases. Optionally, any of the constructs described herein can include one or more introns, for example, between the promoter sequence and a nucleic acid encoding a polypeptide sequence (e.g., an ABE), to facilitate expression of one or more polypeptides sequences in the construct.

TABLE 2
Plasmids

| No. | Name | Purpose | Generation strategy |
|---|---|---|---|
| 1 | pspCas9-MS2- | Plasmid expressing SpCas9 mRNA with | The annealed product of sp-loop1F and sp- |
| | 3′UTR-sgRNA- | a MS2 aptamer and 2xHBB 3′UTR, and | loop1R was inserted between HindIII and |
| | 2xMS2 vector | guide RNA with MS2 replacing the | EcoRI sites of pU6-sgRosa26-1_CBh-Cas9- |
| | | Tetraloop and the loop of stem loop 2. | T2A-BFP (Addgene 64216) by infusion |
| | | This plasmid comprises: | reaction to obtain pspCas9-1loop. The 600 |
| | | (1) an expression cassette comprising | bp FseI and EagI fragment from pSaCas9- |
| | | SEQ ID NO: 34 (a CMV promoter); | 1xms2-2x3′UTR (Addgene 122946) was |
| | | SEQ ID NO: 48 (intron); a nucleic acid | used to replace the FseI and NotI fragment of |
| | | comprising SEQ ID NO: 29, which | pspCas9-1loop by DNA ligation to obtain |
| | | encodes SEQ ID NO: 30 (spCAs9 | pSpCas9-1loop-3′UTR. Then a AflIII- |
| | | (D10A); SEQ ID NO: 10 (MS2 | Acc65I synthesized DNA fragment |
| | | aptamer); and SEQ ID NO: 31 (2 X HBB | (GenScript, |
| | | 3′UTR) | ACATGTgagggcctatttcccatgattccttcatatttgcat |
| | | (2) an expression cassette comprising | atacgatacaaggctgttagagagataattggaattaatttgact |
| | | SEQ ID NO: 32 (U6 promoter) and SEQ | gtaaacacaaagatattagtacaaaatacgtgacgtagaaagt |
| | | ID NO: 33 (sgRNA with MS2 aptamer | aataatttcttgggtagtttgcagttttaaaattatgttttaaaatgg |
| | | in tetraloop and MS2 aptamer in ST2 | actatcatatgcttaccgtaacttgaaagtatttcgatttcttggct |
| | | loop. | ttatatatcttgtggaaaggacgaaacaccggtgtcttcCTC |
| | | | GAGgaagacccgtttgagagctaggccaacatgaggatca |
| | | | cccatgtctgcagggcctagcaagttcaaataaggctagtccg |
| | | | ttatcaacttggccaacatgaggatcacccatgtctgcagggc |
| | | | caagtggcaccgagtcggtgctttttttGGTACC) was |
| | | | inserted into the AflIII and Acc65I sites of |
| | | | pSpCas9-1loop-3′UTR to obtain pspCas9- |
| | | | MS2-3′UTR-sgRNA-2xMS2 vector. |
| 2 | pspCas9-ABE- | Plasmid expressing ABEmax, with a | The 1.8 kb SnaB 1~ BglII fragment from |
| | MS2-3′UTR- | MS2 aptamer and 2xHBB 3′UTR, and | pCMV_ABEmax (Addgene ID 112095) was |
| | sgRNA-2xMS2 | guide RNA with MS2 replacing the | inserted between the SnaB1~ BglII sites of |
| | | Tetraloop and the loop of stem loop 2 | pspCas9-MS2-3′UTR-sgRNA-2xMS2, so |
| | | This plasmid comprises: | that the DNA coding for ABE and SpCas9 |
| | | (1) an expression cassette comprising | D10A mutation was introduced into the new |
| | | SEQ ID NO: 34 (a CMV promoter); | construct. |
| | | SEQ ID NO: 27, which encodes SEQ ID | |
| | | NO: 28 (ABEMAX), SEQ ID NO: 10 | |
| | | (MS2 aptamer); and SEQ ID NO: 31 (2 | |
| | | X HBB 3′UTR); and | |
| | | (2) an expression cassette comprising | |
| | | SEQ ID NO: 32 (U6 promoter) and SEQ | |
| | | ID NO: 33 (sgRNA with MS2 aptamer | |
| | | in tetraloop and MS2 aptamer in ST2 | |
| | | loop. | |
| 3 | pspCas9-ABE- | Plasmid expressing ABEmax, with | pspCas9-ABE-MS2-3′UTR-sgRNA-2xMS2 |
| | 3′UTR-sgRNA- | 2xHBB 3′UTR, and guide RNA with | was cut with XbaI to remove the MS2 |
| | 2xMS2 | MS2 replacing the Tetraloop and the | aptamer in ABE 3′ UTR, the vector was |
| | | loop of stem loop 2. | ligated by T4 DNA ligase. |
| | | This plasmid comprises: | |
| | | (1) an expression cassette comprising | |
| | | SEQ ID NO: 34 (a CMV promoter); | |
| | | SEQ ID NO: 27, which encodes SEQ ID | |
| | | NO: 28 (ABEMAX); and SEQ ID NO: | |
| | | 31 (2 X HBB 3′UTR); and | |
| | | (2) an expression cassette comprising | |
| | | SEQ ID NO: 32 (U6 promoter) and SEQ | |
| | | ID NO: 33 (sgRNA with MS2 aptamer | |
| | | in tetraloop and MS2 aptamer in ST2 | |
| | | loop. | |
| 4 | pspCas9-ABE- | Plasmid expressing ABEmax and ABE- | The annealed products of ABE-g1- |
| | 3′UTR-sgRNA- | g1 sgRNA, the sgRNA has MS2 | F (ACCGGAACACAAAGCATAGACTGC) |
| | 2xMS2-ABE-g1 | replacing the Tetraloop and the loop of | (SEQ ID NO: 83) and ABE-g1- |
| | | stem loop 2 | R (AAACGCAGTCTATGCTTTGTGTTC) |
| | | This plasmid comprises: | (SEQ ID NO: 84)was inserted between the |
| | | (1) an expression cassette comprising | two BbsI sites of pspCas9-ABE-3′UTR- |
| | | SEQ ID NO: 34 (a CMV promoter); | sgRNA-2xMS2 by T4 DNA ligase. |
| | | SEQ ID NO: 27, which encodes SEQ ID | |
| | | NO: 28 (ABEMAX); and SEQ ID NO: | |
| | | 31 (2 X HBB 3′UTR); and | |
| | | (2) an expression cassette comprising | |
| | | SEQ ID NO: 32 (U6 promoter) and SEQ | |
| | | ID NO: 35 (ABE-gl sgRNA with MS2 | |
| | | aptamer in tetraloop and MS2 aptamer in | |
| | | ST2 loop. | |
| 5 | pspCas9-ABE- | Plasmid expressing ABEmax and ABE- | The annealed products of ABE-g2- |
| | 3′UTR-sgRNA- | g2 sgRNA, the sgRNA has MS2 | F (ACCGGAGTATGAGGCATAGACTGC) |
| | 2xMS2-ABE-g2 | replacing the Tetraloop and the loop of | (SEQ ID NO: 85) and ABE-g2- |
| | | stem loop 2. | R (AAACGCAGTCTATGCCTCATACTC) |
| | | This plasmid comprises: | (SEQ ID NO: 86)was inserted between the |
| | | (1) an expression cassette comprising | two BbsI sites of pspCas9-ABE-3′UTR- |
| | | SEQ ID NO: 34 (a CMV promoter); | sgRNA-2xMS2 by T4 DNA ligase. |
| | | SEQ ID NO: 27, which encodes SEQ ID | |
| | | NO: 28 (ABEMAX); and SEQ ID NO: | |
| | | 31 (2 X HBB 3′UTR); and | |
| | | (2) an expression cassette comprising | |
| | | SEQ ID NO: 32 (U6 promoter) and SEQ | |
| | | ID NO: 36 (ABE-g2 sgRNA with MS2 | |
| | | aptamer in tetraloop and MS2 aptamer in | |
| | | ST2 loop. | |
| 6 | pspCas9-ABE- | Plasmid expressing ABEmax and ABE- | The annealed products of ABE-g5- |
| | 3′UTR-sgRNA- | g2 sgRNA, the sgRNA has MS2 | F (ACCGGATGAGATAATGATGAGTCA) |
| | 2xMS2-ABE-g5 | replacing the Tetraloop and the loop of | (SEQ ID NO: 87)and ABE-g5-R |
| | | stem loop 2. | (aaacTGACTCATCATTATCTCATC) (SEQ |
| | | This plasmid comprises: | ID NO: 88) was inserted between the two |
| | | (1) an expression cassette comprising | BbsI sites of pspCas9-ABE-3′UTR-sgRNA- |
| | | SEQ ID NO: 34 (a CMV promoter); | 2xMS2 by T4 DNA ligase. |
| | | SEQ ID NO: 27, which encodes SEQ ID | |
| | | NO: 28 (ABEMAX); and SEQ ID NO: | |
| | | 31 (2 X HBB 3′UTR); and | |
| | | (2) an expression cassette comprising | |
| | | SEQ ID NO: 32 (U6 promoter) and SEQ | |
| | | ID NO: 37 (ABE-g5 sgRNA with MS2 | |
| | | aptamer in tetraloop and MS2 aptamer in | |
| | | ST2 loop. | |
| 7 | pspCas9-ABE- | Plasmid expressing ABEmax, with a | The 400 bp AflIII~Acc65I fragment from |
| | 3′UTR-sgRNA- | 2xHBB 3′UTR, and guide RNA with | pspCas9-3′UTR-Tetra-com vector was |
| | Tetra-com | com replacing the Tetraloop. | inserted between AflIII~Acc65I sites of |
| | vector | This plasmid comprises: | pspCas9-ABE-3′UTR-sgRNA-2xMS2 by T4 |
| | | (1) an expression cassette comprising | DNA ligation. |
| | | SEQ ID NO: 34 a CMV promoter); | |
| | | SEQ ID NO: 27, which encodes SEQ ID | |
| | | NO: 28 (ABEMAX); and SEQ ID NO: | |
| | | 31 (2 X HBB 3′UTR); and | |
| | | (2) an expression cassette comprising | |
| | | SEQ ID NO: 32 (U6 promoter) and SEQ | |
| | | ID NO: 38 (sgRNA with com aptamer in | |
| | | tetraloop). | |
| 8 | pspCas9-ABE- | Plasmid expressing ABEmax, with a | The annealed products of ABE-g1- |
| | 3′UTR-sgRNA- | 2xHBB 3′UTR, and guide RNA with | F (ACCGGAACACAAAGCATAGACTGC) |
| | Tetra-com-ABE- | com replacing the Tetraloop. | (SEQ ID NO: 89) and ABE-g1- |
| | gl | This plasmid comprises: | R (AAACGCAGTCTATGCTTTGTGTTC) |
| | | (1) an expression cassette comprising | (SEQ ID NO: 90) was inserted between the |
| | | SEQ ID NO: 34 (a CMV promoter); | two BbsI sites of pspCas9-ABE-3′UTR- |
| | | SEQ ID NO: 27, which encodes SEQ ID | sgRNA-Tetra-com vector by T4 DNA |
| | | NO: 28 (ABEMAX); and SEQ ID NO: | ligase. |
| | | 31 (2 X HBB 3′UTR); and | |
| | | (2) an expression cassette comprising | |
| | | SEQ ID NO: 32 (U6 promoter) and SEQ | |
| | | ID NO: 39 (ABE-gl sgRNA with com | |
| | | aptamer in tetraloop). | |
| 9 | pspCas9-ABE- | Plasmid expressing ABEmax, with a | The annealed products of ABE-g2- |
| | 3′UTR-sgRNA- | 2xHBB 3′UTR, and ABE-g2guide RNA | F (ACCGGAGTATGAGGCATAGACTGC) |
| | Tetra-com-ABE- | with com replacing the Tetraloop. | (SEQ ID NO: 91) and ABE-g2- |
| | g2 | This plasmid comprises: | R (AAACGCAGTCTATGCCTCATACTC) |
| | | (1) an expression cassette comprising | (SEQ ID NO: 92) was inserted between the |
| | | SEQ ID NO: 34 (a CMV promoter); | two BbsI sites of pspCas9-ABE-3′UTR- |
| | | SEQ ID NO: 27, which encodes SEQ ID | sgRNA-Tetra-com vector by T4 DNA |
| | | NO: 28 (ABEMAX); and SEQ ID NO: | ligase. |
| | | 31 (2 X HBB 3′UTR); and | |
| | | (2) an expression cassette comprising | |
| | | SEQ ID NO: 32 (U6 promoter) and SEQ | |
| | | ID NO: 40 (ABE-g2 sgRNA with com | |
| | | aptamer in tetraloop). | |
| 10 | pspCas9-ABE- | Plasmid expressing ABEmax, with a | The annealed products of ABE-g5- |
| | 3′UTR-sgRNA- | 2xHBB 3′UTR, and ABE-g5 guide RNA | F (ACCGGATGAGATAATGATGAGTCA) |
| | Tetra-com-ABE- | with com replacing the Tetraloop. | (SEQ ID NO: 93) and ABE-g5-R |
| | g5 | This plasmid comprises: | (aaacTGACTCATCATTATCTCATC) (SEQ |
| | | (1) an expression cassette comprising | ID NO: 94) was inserted between the two |
| | | SEQ ID NO: 34 (a CMV promoter); | BbsI sites of pspCas9-ABE-3′UTR-sgRNA- |
| | | SEQ ID NO: 27, which encodes SEQ ID | Tetra-com vector by T4 DNA ligase. |
| | | NO: 28 (ABEMAX); and SEQ ID NO: | |
| | | 31 (2 X HBB 3′UTR); and | |
| | | (2) an expression cassette comprising | |
| | | SEQ ID NO: 32 (U6 promoter) and SEQ | |
| | | ID NO: 41 (ABE-g5 sgRNA with com | |
| | | aptamer in tetraloop). | |
| 11 | pspCas9-ABE- | Plasmid expressing ABEmax, with a | The PCR product from pspCas9-3′UTR- |
| | 3′UTR-sgRNA- | 2xHBB 3′UTR, and unmodified ABE-g5 | IL2RG with primers g5-ST-com-F |
| | g5 | guide RNA. | (aaggacgaaacaccgGATGAGATAATGATG |
| | | This plasmid comprises: | AGTCAGTTTGAGAGCTAg) (SEQ ID NO: |
| | | (1) an expression cassette comprising | 95) and ABE-ST-com-R |
| | | SEQ ID NO: 34 (a CMV promoter); | (TTATGTAACGGGTACCAAAA) (SEQ ID |
| | | SEQ ID NO: 27, which encodes SEQ ID | NO: 96) was inserted between the |
| | | NO: 28 (ABEMAX); and SEQ ID NO: | BbsI~Acc65I sites of pspCas9-ABE-3′UTR- |
| | | 31 (2 X HBB 3′UTR); and | sgRNA-Tetra-com-vector by infusion |
| | | (2) an expression cassette comprising | reaction. |
| | | SEQ ID NO: 32 (U6 promoter) and SEQ | |
| | | ID NO: 42 (umodified ABE-g5 sgRNA) | |
| 12 | pspCas9-ABE- | Vector plasmid expressing ABEmax, | The PCR product from pspCas9-3′UTR- |
| | 3′UTR-sgRNA- | with a 2xHBB 3′UTR, and guide RNA | IL2RG with primers ST-com-F |
| | ST2-com vector | with com replacing the ST2 loop. | (aaggacgaaacaccggtgtcttcCTCGAGgaagaccc |
| | | This plasmid comprises: | GTTTGAGAGCTAg) (SEQ ID NO: 97) and |
| | | (1) an expression cassette comprising | ABE-ST-com-R |
| | | SEQ ID NO: 34 (a CMV promoter); | (TTATGTAACGGGTACCAAAA) (SEq ID |
| | | SEQ ID NO: 27, which encodes SEQ ID | NO: 98) was inserted between the |
| | | NO: 28 (ABEMAX); and SEQ ID NO: | BbsI~Acc65I sites of pspCas9-ABE-3′UTR- |
| | | 31 (2 X HBB 3′UTR); and | sgRNA-Tetra-com-vector by infusion |
| | | (2) an expression cassette comprising | reaction. |
| | | SEQ ID NO: 32 (U6 promoter) and SEQ | |
| | | ID NO: 43 (sgRNA with com replacing | |
| | | ST2 loop). | |
| 13 | pspCas9-ABE- | Plasmid expressing ABEmax, with a | The annealed products of ABE-gl- |
| | 3′UTR-sgRNA- | 2xHBB 3′UTR, and ABE-glguide RNA | F (ACCGGAACACAAAGCATAGACTGC) |
| | ST2-com-ABE- | with com replacing the ST2 loop. | (SEQ ID NO: 99) and ABE-gl- |
| | g1 | This plasmid comprises: | R (AAACGCAGTCTATGCTTTGTGTTC) |
| | | (1) an expression cassette comprising | (SEQ ID NO: 100) was inserted between the |
| | | SEQ ID NO: 34 (a CMV promoter); | two BbsI sites of pspCas9-ABE-3′UTR- |
| | | SEQ ID NO: 27, which encodes SEQ ID | sgRNA-ST2-com vector by T4 DNA ligase. |
| | | NO: 28 (ABEMAX); and SEQ ID NO: | |
| | | 31 (2 X HBB 3′UTR); and | |
| | | (2) an expression cassette comprising | |
| | | SEQ ID NO: 32 (U6 promoter) and SEQ | |
| | | ID NO: 44 (ABE-gl sgRNA with com | |
| | | replacing ST2 loop). | |
| 14 | pspCas9-ABE- | Plasmid expressing ABEmax, with a | The annealed products of ABE-g2- |
| | 3′UTR-sgRNA- | 2xHBB 3′UTR, and ABE-g2 guide RNA | F (ACCGGAGTATGAGGCATAGACTGC) |
| | ST2-com-ABE- | with com replacing the ST2 loop. | (SEQ ID NO: 101) and ABE-g2- |
| | g2 | This plasmid comprises: | R (AAACGCAGTCTATGCCTCATACTC) |
| | | (1) an expression cassette comprising | (SEQ ID NO: 102) was inserted between the |
| | | SEQ ID NO: 34 (a CMV promoter); | two BbsI sites of pspCas9-ABE-3′UTR- |
| | | SEQ ID NO: 27, which encodes SEQ ID | sgRNA-ST2-com vector by T4 DNA ligase. |
| | | NO: 28 (ABEMAX); and SEQ ID NO: | |
| | | 31 (2 X HBB 3′UTR); and | |
| | | (2) an expression cassette comprising | |
| | | SEQ ID NO: 32 (U6 promoter) and SEQ | |
| | | ID NO: 45 (ABE-g2 sgRNA with com | |
| | | replacing ST2 loop). | |
| 15 | pspCas9-ABE- | Plasmid expressing ABEmax, with a | The PCR product from pspCas9-3′UTR-ST2- |
| | 3′UTR-sgRNA- | 2xHBB 3′UTR, and ABE-g5 guide RNA | com-IL2RG with primers g5-ST-com-F |
| | ST2-com-ABE- | with com replacing the ST2 loop. | (aaggacgaaacaccgGATGAGATAATGATG |
| | g5 | This plasmid comprises: | AGTCAGTTTGAGAGCTAg) (SEQ ID NO: |
| | | (1) an expression cassette comprising | 103) and ABE-ST-com-R |
| | | SEQ ID NO: 34 (a CMV promoter); | (TTATGTAACGGGTACCAAAA) (SEQ ID |
| | | SEQ ID NO: 27, which encodes SEQ ID | NO: 104) was inserted between the |
| | | NO: 28 (ABEMAX); and SEQ ID NO: | BbsI~Acc65I sites of pspCas9-ABE-3′UTR- |
| | | 31 (2 X HBB 3′UTR); and | sgRNA-Tetra-com-vector by infusion |
| | | (2) an expression cassette comprising | reaction. |
| | | SEQ ID NO: 32 (U6 promoter) and SEQ | |
| | | ID NO: 46 (ABE-g5 sgRNA with com | |
| | | replacing ST2 loop). | |
| 16 | pspCas9-ABE- | Plasmid expressing ABEmax, with a | The PCR product from pspCas9-3′UTR- |
| | 3′UTR-sgRNA | 2xHBB 3′UTR, and sgRNA scaffold | IL2RG with primers g5-ST-com-F |
| | vector | without modification. | (aaggacgaaacaccgGATGAGATAATGATG |
| | | This plasmid comprises: | AGTCAGTTTGAGAGCTAg) (SEQ ID NO: |
| | | (1) an expression cassette comprising | 105) and ABE-ST-com-R |
| | | SEQ ID NO: 34 (a CMV promoter); | (TTATGTAACGGGTACCAAAA) (SEQ ID |
| | | SEQ ID NO: 27, which encodes SEQ ID | NO: 106) was inserted between the |
| | | NO: 28 (ABEMAX); and SEQ ID NO: | BbsI~Acc65I sites of pspCas9-ABE-3′UTR- |
| | | 31 (2 X HBB 3′UTR); and | sgRNA-Tetra-com-vector by infusion |
| | | (2) an expression cassette comprising | reaction. |
| | | SEQ ID NO: 32 (U6 promoter) and SEQ | |
| | | ID NO: 47 (unmodified sgRNA) | |

TABLE 3
Primers

| Primer name | Sequence | Use |
|---|---|---|
| USP38-F1 | atggccacagatttcaggag (SEQ ID NO: 49) | To amplify the 444 bp cDNA region |
| USP38-R1 | ggcttccactttttgtgagg (SEQ ID NO: 50) | containing the putative ABE hotspot |
| USP38-F3 | Aggcctcacacaagccttc (SEQ ID NO: 51) | To amplify the genomic DNA region or the |
| | | cDNA region containing the putative ABE |
| | | hotspot. Used with USP38R1. |
| ABE-g1-onF | ACCTGGCTGAGCTAACTGTG | To amplify ABE g1 target for NGS |
| | (SEQ ID NO: 52) | |
| ABE-g1-onR | TCCAGCCCCATCTGTCAAAC | |
| | (SEQ ID NO: 53) | |
| ABE-g2-onF | GGAACCTCAGGTGAAAAGTCCA | To amplify ABE g2 target for NGS |
| | (SEQ ID NO: 54) | |
| ABE-g2-onR | ACTTCCTGAAATGCTGTGCG | |
| | (SEQ ID NO: 55) | |
| ABE-g5-onF | GTCTGAGGTCACACAGTGGG | To amplify ABE g2 target for NGS |
| | (SEQ ID NO: 56) | |
| ABE-g5-onR | CTGAGAGCAGGGACCACATC | |
| | (SEQ ID NO: 57) | |
| g1-ABE-R | CCCGCAGTCTATGCTTCGC | For qPCR to detect base editing at ABE site |
| | (SEQ ID NO: 58) | 1 with ABE-g1-onF |
| g2-ABE-R | CCTGCAGTCTATGCCTCAC | For qPCR to detect base editing at ABE site |
| | (SEQ ID NO: 59) | 2 with ABE-g2-onF |
| g5-ABE-R | AGCCCTGACTCATCATTACCC | For qPCR to detect base editing at ABE site |
| | (SEQ ID NO: 60) | 5 with ABE-g5-onF |
| HEK2-F | TTGCACTGCCATTCTACCAA | To amplify the HEK2 region for in vitro |
| | (SEQ ID NO: 61) | assay of ABE RNP activity |
| HEK2-R | ATCCACAGCAACACCCTCTC | |
| | (SEQ ID NO: 62) | |
| ABE-g5-F | ACCGGATGAGATAATGATGAGTCA | For qPCR to detect sgRNA |
| | (SEQ ID NO: 63) | |
| Sp-sgRNA-R1 | Gcaccgactcggtgccactt (SEQ ID NO: 64) | |

TABLE 4
Target sequences and oligos for cloning guides into sgRNA-expressing vectors

| | Target sequence | sgRNA | Forward Oligo for | Reverse Oligo for |
|---|---|---|---|---|
| Target name | with PAM | name | cloning | cloning |
| ABE site 1 | GAACACAAAGCATAG | ABE-g1 | ACCGGAACACAAA | AAACGCAGTCTAT |
| | ACTGCGGG | | GCATAGACTGC | GCTTTGTGTTC |
| | (SEQ ID NO: 65) | | (SEQ ID NO: 68) | (SEQ ID NO: 71) |
| ABE site 2 | GAGTATGAGGCATAG | ABE-g2 | ACCGGAGTATGAG | AAACGCAGTCTAT |
| | ACTGCAGG | | GCATAGACTGC | GCCTCATACTC |
| | (SEQ ID NO: 66) | | (SEQ ID NO: 69) | (SEQ ID NO: 72) |
| ABE site 5 | GATGAGATAATGATG | ABE-g5 | ACCGGATGAGATA | aaacTGACTCATCAT |
| | AGTCAGGG | | ATGATGAGTCA | TATCTCATC |
| | (SEQ ID NO: 67) | | (SEQ ID NO: 70) | (SEQ ID NO: 73) |

### ABE 7.10 Expression and Purification

The SNU-ABE plasmid, which encodes codon optimized ABE 7.10 linked to an N-terminal His tag, was first transformed into BL21-star (DE3) competent cells, which were then plated on a Luria-Bertani (LB)-agar plate containing 50 μg ml⁻¹ kanamycin. After incubation overnight at 37° C., a single colony was selected and grown overnight at 37° C. (pre-culture) in LB broth containing 50 μg ml⁻¹ kanamycin and 10 μM ZnCl₂ to maintain ABE catalytic activity. Following this pre-culture, part of the inoculant was transferred to several 400 ml LB media, in 1 L flask, for large culture (up to 6 L), and the resulting culture was incubated at 37° C. with shaking at 250 rpm until the absorbance A600=˜0.5-0.70. Next, the culture was put on ice for about 1 h. To induce ABE protein expression, 1 mM isopropyl β-D-1-thiogalactopyranoside (GoldBio, St. Louis, MO) was added and the culture was incubated at 18° C., for 14-16 h, with 250 rpm shaking.

The later steps in the purification procedure were all carried out at 0-4° C. Prior to cell lysis, the cells were harvested by centrifugation at 5,000 g for 10 min, after which they were resuspended in 8 ml lysis buffer per 400 ml inoculants [50 mM sodium phosphate (Sigma-Aldrich, St. Louis, MO), 500 mM 1% Triton X-100 (Sigma-Aldrich), 20% glycerol, 1 mM phenylmethylsulfonyl fluoride (Sigma-Aldrich), 1 mg ml-1 lysozyme from chicken egg white (Sigma-Aldrich), 10 μM ZnCl2 (Sigma-Aldrich), pH 8.0]. For lysis, cells were frozen in liquid nitrogen and thawed at 37° C. for a total of three times. For further lysis, cells were sonicated (3 min total, 5 s on, 10 s off), after which they were centrifuged at 13,000 rpm to clear the lysate. The supernatant was mixed with 10 ml Ni-NTA agarose beads (QIAGEN) and the resin-lysate mixture was gently rotated for 1 h and then loaded onto a column. The column was washed three times each with 50 ml nickel wash buffer [50 mM sodium phosphate (Sigma-Aldrich), 150 mM NaCl (Sigma-Aldrich), 35 mM imidazole (Sigma-Aldrich), 1 mM DTT (GoldBio), 10 μM ZnCl2 (Sigma-Aldrich), pH 8.0] and then the proteins were eluted with 20 ml nickel elution buffer (50 mM sodium phosphate, 150 mM NaCl, 250 mM imidazole, 20% glycerol, 1 mM DTT, 10 μM ZnCl₂, pH 8.0). The eluted proteins were further purified with 5 ml heparin Sepharose beads (GE Healthcare) in another column. The column was washed with 50 ml heparin wash buffer (50 mM sodium phosphate, 150 mM NaCl, 1 mM DTT, 10 μM ZnCl2, pH 8.0) three times and proteins were eluted with 20 ml heparin elution buffer (50 mM sodium phosphate, 750 mM NaCl, 20% glycerol, 1 mM DTT, 10 μM ZnCl2, pH 8.0). Finally, the eluted proteins were concentrated and the buffer changed to ABE storage buffer (200 mM NaCl, 20 mM HEPES, 1 mM DTT, 40% glycerol, PH 7.5) by centrifugation through an Amicon Ultra-4 column with a 100,000 kDa cutoff (Millipore) at 6,000×g.

### ABE- and Endo V-Mediated In Vitro Digestion of Amplified Target Site

The region spanning the ABE site 1 (Hek2) was amplified using polymerase chain reaction (PCR, chr5:+87944480-87944802) with primers HEK2-F and HEK2-R. 2 μg of the resulting amplicon was then incubated with 4 μg ABE 7.10 protein and 3 μg sgRNA (targeting ABE site 1) in 200 μl ABE reaction buffer [50 mM Tris-HCl (Sigma-Aldrich), 25 mM KCl (Sigma-Aldrich), 2.5 mM MgSO4 (Sigma-Aldrich), 0.1 mM Ethylenediaminetetraacetic acid (EDTA: Sigma-Aldrich), 2 mM DTT (GoldBio), 10 mM ZnCl2 (Sigma-Aldrich), 20% glycerol] at 37° C. for 1-2 h. Following the reaction, ABE protein and sgRNA were removed by incubation with 80 μg Proteinase K and 400 μg RNase A (both from Qiagen), respectively, for 10 min. The amplicons were purified using a PCR purification kit (MGmed). 1 μg of the purified amplicons were incubated with 10 units of Endo V enzyme (NEB) for 1 h. Next, the mixture was incubated with 80 μg Proteinase K, and again purified with a PCR purification kit (MGmed). Finally, the DNA fragments were imaged following electrophoresis on a 2% agarose gel.

### RNP Electroporation

CRISPR RNA for ABE site 1 (rGrArArCrArCrArArArGrCrArUrArGrArCrUrGrCrGrUrUrUrUrArGrArGrCrUrArUrGrCr U) (SEQ ID NO: 74) was synthesized by IDT Inc. (Coralville, IA). Alt-R® CRISPR-Cas9 tracrRNA, Alt-R® CRISPR-Cas9 Negative Control crRNA, Alt-R® Cas9 Electroporation Enhancer, and Nuclease Free Duplex Buffer were purchased from IDT Inc. RNP reconstitution and electroporation were performed following the IDT Inc. instructions. A total of 2×10⁵ HEK293T cells were used for each electroporation with the Amaxa Nucleofector system (Lonza, Basel, Switzerland). The cells were re-suspended in 100 μl of nucleofection buffer from the Cell Line Nucleofector™ Kit V (Catalog #VCA-1003, Lonza), and placed in the electroporation cuvette. Then 1 μl of Alt-R® Cas9 Electroporation Enhancer and 5 μl of reconstituted ABE RNPs were added to the cells in the cuvette. Finally, the cells were given an electrical shock with protocol Q-001. The cells were removed from the cuvette and cultured in growth medium for 24 hours before analysis.

### Lentiviral Capsid-RNP Production

Lentiviral capsids packaged with ABE RNPs were produced by a three plasmid transfection procedure. Briefly, 13 million HEK293T cells were cultured in a 15-cm dish with 15 ml Opti-MEM. 16 μg of ABP-modified packaging plasmid pspAX2-D64V-NC-ABP (ABP can be MCP (MS2 coat protein, binding to RNA aptamer MS2) (Peabody et al., Nucleic Acids Res 1992, 20 (7): 1649-55) or Com (binding to RNA aptamer com)) (Hattman et al., P Natl Acad Sci USA 1991, 88 (22):10027-10031), 6 μg envelope plasmid (pMD2.G), and 16 μg plasmid DNA co-expressing ABE, and the corresponding aptamer-modified sgRNA were mixed in 1 ml Opti-MEM. 76 ul of 1 mg/ml polyethylenimine (PEI, Polysciences Inc., Bellevue, WA) was mixed in 1 ml Opti-MEMO Reduced-Serum Medium. The DNA mixture and the PEI mixture were then mixed and incubated at room temperature for 15 mins. The DNA/PEI mixture was then added to the cells in Opti-MEMO medium. 24 h after transfection, the medium was changed into 15 ml Opti-MEMO medium and the ABE RNP laden virus-like particles (VLP) were collected 48 h and 72 h after transfection. The supernatant was spun for 10 min at 500 g to remove cell debris. The cleared supernatant can be used directly or be further concentrated as described below. Transfection can also be done in 10-cm dishes or 6-well plates with Fugene HD (Promega, Madison, WI). DNA amounts were proportionally scaled based on vessel surface area.

### Concentrating ABE RNP-Laden VLPs

The supernatant containing ABE RNP-laden VLPs was concentrated with the KrosFlo® Research 2i (KR2i) Tangential Flow Filtration System (Spectrum Lab, Cat. No. SYR2-U20) using the concentration-diafiltration-concentration mode. Briefly, 150-300 ml supernatant was first concentrated to about 50 ml, diafiltrated with 500 ml to 1000 ml PBS, and finally concentrated to about 8 ml. The hollow fiber filter modules were made from modified polyethersulfone, with a molecular weight cut-off of 500 kDa. The flow rate and the pressure limit were 80 ml/min and 8 psi for the filter module D02-E500-05-N, and 10 ml/min and 5 psi for the filter module C02-E500-05-N. Capsid-RNPs were also concentrated by ultracentrifugation, as described previously (Lu et al., Nucleic Acids Res 2019, 47 (8): e44.)

### VLP Quantification

Concentration of VLPs was determined by p24 (lentiviral capsid protein CA) based ELISA (Cell Biolabs, QuickTiter™ Lentivirus Titer Kit Catalog Number VPK-107, San Diego, CA). When un-concentrated samples were assayed, the VLPs were precipitated according to the manufacturer's instructions so that the soluble p24 protein was not detected.

### Western Blotting Analysis of Capsid and Cas9 Proteins in ABE RNP VLPs

200 ng p24 of VLPs were transiently treated with 0.5% Triton X-100 following a published procedure (Wiegers et al., J Virol 1998, 72 (4): 2846-54). Briefly, VLPs were centrifuged with a Sorvall T-890 rotor (2 h at 120,000 g) through step gradients containing a 1 ml layer of 10% sucrose in STE [100 mM NaCl, 50 mM Tris/HCl (pH 7.5), 1 mM EDTA] with or without 0.5% Triton X-100, and a cushion of 2 ml 20% sucrose in STE solution. The pelleted VLP particles were directly lysed in 100 μl of 1× Laemmli sample buffer for Western blotting or for purifying RNA for RT-qPCR analysis.

The proteins in each sample were separated on SDS-PAGE gels and analyzed by Western blotting. The antibodies used include mouse monoclonal anti-SpCas9 antibody (ThermoFisher, CRISPR-Cas9 Monoclonal Antibody 7A9-3A3, Catalog #MA1-201, 1:1000), and p24 mouse monoclonal antibody for capsid protein (Cell Biolabs, Cat No. 310810, 1:1000). HRP-conjugated anti-Mouse IgG (H+L) (ThermoFisher Scientific, Waltham, MA, Cat No. 31430, 1:5000) and HRP-conjugated anti-Rabbit IgG (H+L) (ThermoFisher, Cat No. 31460, 1:5000) secondary antibodies were used in Western blotting. SpCas9 RNP standards were GenCrispr NLS-Cas9-NLS Nuclease from GenScript (Piscataway, NJ, Cat #Z033895). Chemiluminescent reagents (Pierce, Dallas, TX) were used to visualize the protein signals in the LAS-3000 system (Fujifilm, Tokyo, Japan). Densitometry (NIH ImageJ software) was used to quantify protein amounts.

### RNA Isolation and RT-qPCR Analysis

A miRNeasy Mini Kit (QIAGEN, Hilden, Germany, Cat No. 217004) was used to isolate RNA from concentrated capsids or cells. The QuantiTect Reverse Transcription Kit (QIAGEN) was used to reverse-transcribe the RNA to cDNA. For sgRNA reverse transcription, 0.6 μl random primers provided in the kit and 0.4 μl sgRNA-specific primer (Sp-sgRNA-R1, gcaccgactcggtgccactt (SEQ ID NO: 82), 20 μM) were used for reverse transcription. Then guide specific forward primer ABE-g5-F (Table 2) were used together with Sp-sgRNA-R1 in SybrGreen based RT-qPCR to detect sgRNA. Quantitative PCR was run on a QuantStudio™ 3 instrument (Thermo Fisher) or an ABI 7500 instrument (Thermo Fisher).

### VLP Transduction

VLPs (in the amount of about 10-300 ng p24 protein were added to 2.5×10⁴ cells grown in 24-well plates, with 8 μg/ml polybrene. Unconcentrated supernatant of VLPs was diluted with fresh medium at a 1:1 ratio to transduce cells. The cells were incubated with the VLP-containing medium for 12-24 hours, after which the medium was replaced with normal medium.

### Examination of ABE Protein Degradation in Cells

2×10⁴ HEK293T cells were transduced with 100 ng p24 of VLPs containing ABE RNPs with or without aptamer. 12 hours after transduction, the cells were maintained in DMEM with 0.5% FBS to limit cell division. Fresh medium was changed every 48 hours. Cells were collected every 12 hours after transduction to detect the presence of ABE protein by Western blotting, using anti-SpCas9 (Thermo Fisher, Catalog #MA1-201) and anti-0 actin (Sigma, A5441, 1:5000) antibodies. The relative expression of ABE was quantified by densitometry with NIH ImageJ software (Version 1.49). The densitometry data were used to determine protein half-life using the two-phase decay method of GraphPad Prism 5.0 (Graphpad, San Diego, CA).

### Next-Generation Sequencing and Data Analysis

The regions and primers used to amplify target DNA for next generation sequencing are listed in Table 4. The proofreading HotStart® ReadyMix from KAPA Biosystems (Wilmington, MA) was used for PCR. The amplicons were sequenced by GeneWiz's Amplicon-EZ service. Usually 50,000 reads/amplicon were obtained. Base editing was analyzed with the online software BE analyzer (Hwang et al., BMC Bioinformatics 2018, 19 (1): 542) and CRISPRESSO2 (Clement et al., Nat Biotechnol 2019, 37 (3): 224-22), which gave similar results.

### Statistical Analysis

GraphPad Prism software (version 5.0) was used for statistical analyses. T-tests were used to compare the averages of two groups. Analysis of variance (ANOVA) was performed followed by Tukey post hoc tests to analyze data from more than two groups. Bonferroni post hoc tests were performed following ANOVA in cases of two factors. p<0.05 was regarded as statistically significant.

### Results

### RNA Hotspots for Detecting ABE RNA Off-Target Activities

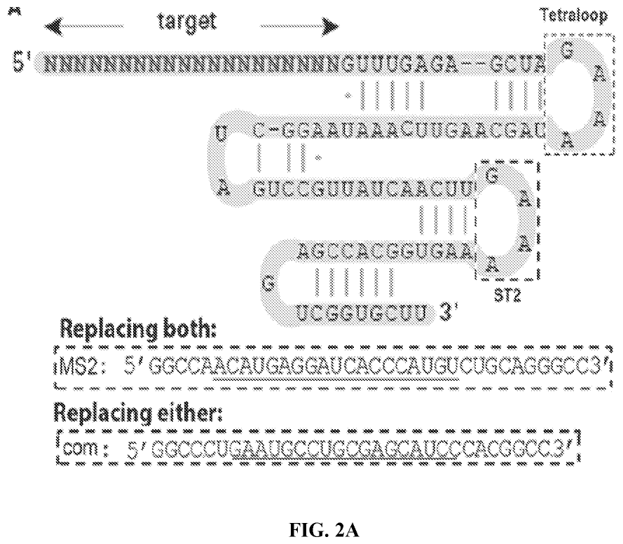

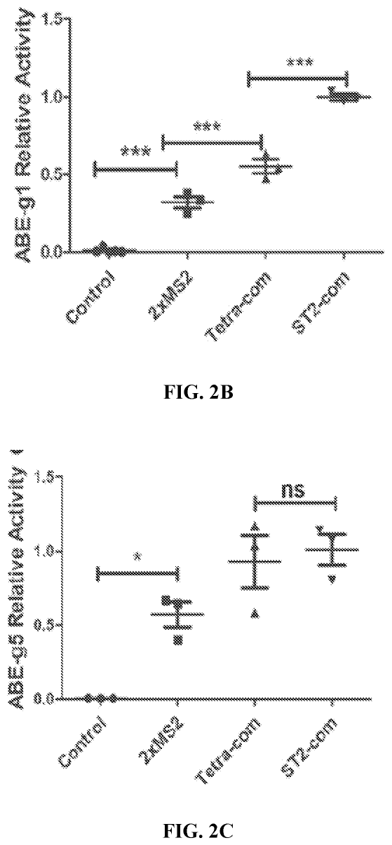

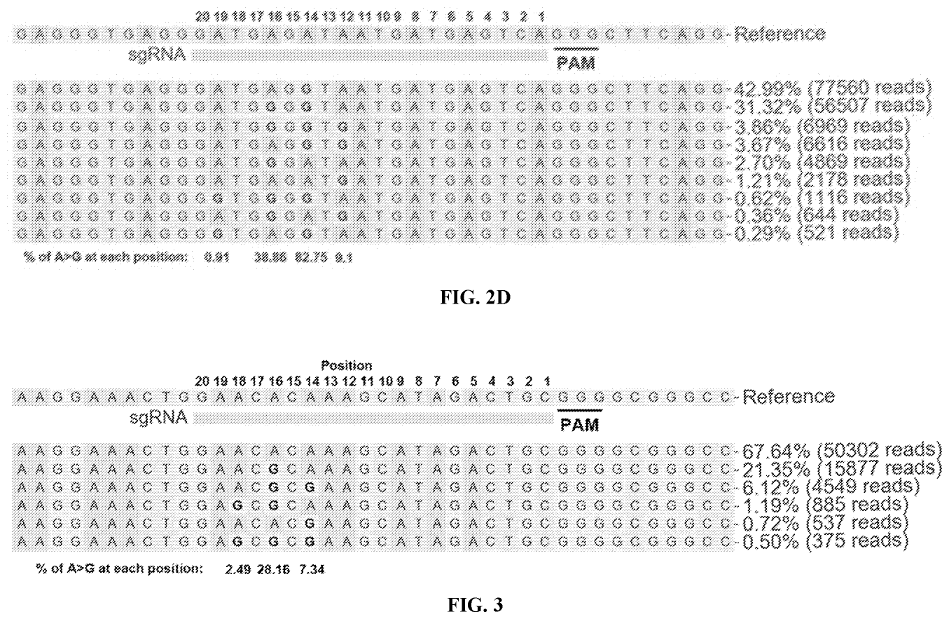

The major goal of this study was to find an ABE delivery method with short activity duration and minimal RNA off-target activities, for which a sensitive RNA off-target detection method is useful. Currently, high-depth RNA sequencing is used to detect ABE RNA off-targets (Grunewald et al., Nature 2019, 569 (7756): 433-437) which is time-consuming and expensive. Recently it was found that the RNA motif CUACGAA (SEQ ID NO: 75) was the most efficient ABE RNA off-target (Grunewald et al., Nat Biotechnol 2019, 37 (9): 1041-1048). A human sequence database was analyzed, and it was found that the human USP38 gene contains a CTACGAA (SEQ ID NO: 76) sequence in its coding region exon 9 (FIG. 2C). RT-PCR confirmed that this gene was expressed in HEK293T cells. Whether the “CUACGAA” (SEQ ID NO: 75) sequence of USP38 mRNA is an ABE RNA off-target hotspot was analyzed.

HEK293T cells were transfected with plasmid DNA expressing Cas9 nickase (negative control), or plasmid DNA expressing ABE and sgRNA targeting ABE site 1 (Gaudelli et al., Nature 2017, 551 (7681): 464-471). 444 bp of the USP38 cDNA spanning the predicted hotspot (primers F1 and R1 in FIG. 2A) were amplified for targeted next-generation sequencing (NGS). In cells transfected with ABE-expressing DNA, the highest peak of “A” to “G” change, at the predicted hotspot CUACGAA (SEQ ID NO: 75) (˜15% of the underlined A was changed to G), was observed, with multiple lower peaks of <5% throughout the analyzed region (FIG. 2B). Similar peaks were absent in control cells with Cas9 nickase (FIG. 2B).

These “A” to “G” changes must be the results of changes in mRNA, since NGS analysis of corresponding DNA amplified from genomic DNA of cells transfected with ABE and ABE site 1 sgRNA revealed an A to G change in less than 0.02% of alleles. The changes observed in USP38 cDNA were most likely the results of nonspecific RNA editing of adenosine (A) to inosine (I), which was recognized as Guanine (G) in reverse transcription and sequencing. The most frequently observed A to G changes all occurred in the UA motif, consistent with previous observations (Grunewald et al. 2019 Nature; Grunewald et al., 2019 Nat. Biotech.) (FIG. 2C).

Focusing on the A to G changes in the “CUACGAA” (SEQ ID NO: 75) motif, these changes were observed in up to 16.7% reads from cDNA of cells transfected with ABE-expressing DNA, but in 0% reads from cDNA of cells transfected with nickase (Table 5). Importantly, only 3 out of 32025 reads with A to G changes when analyzing gDNA of ABE transfected cells were observed. These data showed that the “CUACGAA” (SEQ ID NO: 75) sequence in USP38 mRNA is indeed a hotspot of ABE RNA off-target, and suggest that analyzing RNA off-targets in this hotspot enables us to compare ABE RNA off-target activities resulting from different delivery methods.

TABLE 5
Analysis of CU/(T)ACGAA (SEQ ID NO:
77) to CU/(T)GCGAA (SEQ ID NO: 78)
changes in USP38 cDNA and gDNA by NGS.

| | cDNA | | | |
|---|---|---|---|---|
| | | ABE- | ABE- | gDNA |
| | Nickase | sample 1 | Sample 2 | ABE^{b} |
| Total reads | 15714 | 13657 | 11549 | 32025 |
| Reads with | 0 | 2096 | 1925 | 3 |
| A-G Change^{a} | | | | |
| % with A-G | 0% | 15.3% | 16.7% | 0.00936% |
| Change | | | | |

^{a}Only reads with CU(/T)ACGAA to CU(/T)GCGAA changes were counted.
^{b}All reads were from one NGS sample.

ABE RNPs Delivered by Electroporation Showed Undetectable RNA Off-Target Activities 24 Hours after Delivery

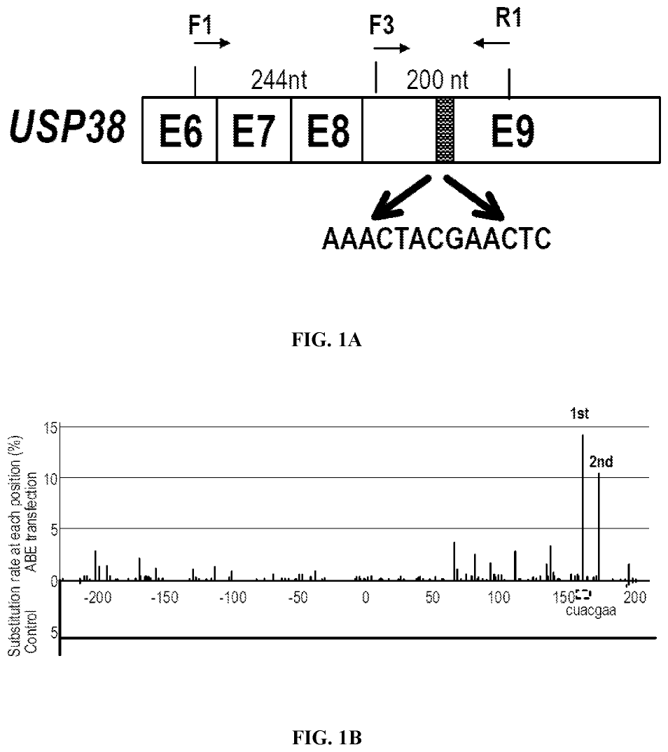

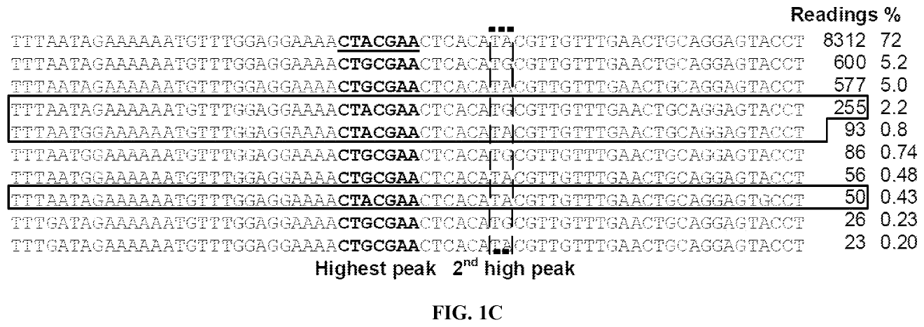

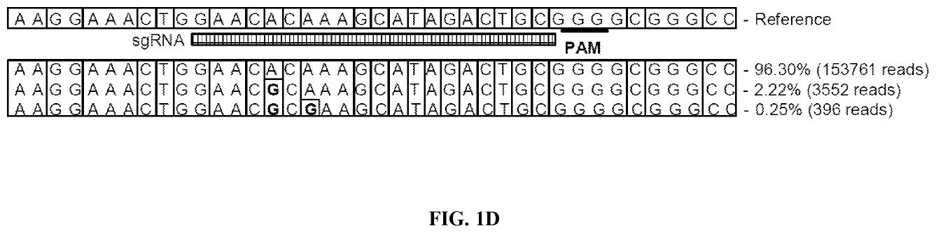

Once an ABE RNA off-target hotspot was confirmed, whether or not delivering ABE RNPs by electroporation showed reduced RNA off-target activity compared with DNA transfection was studied. Recombinant ABE RNPs were prepared, as previously described (Kim et al., Nat Biotechnol 2019, 37 (4), 430-435) and their activities confirmed in an in vitro assay. 10, 5, 2.5, 1.25, and 0.625 μg of ABE RNPs (targeting ABE site 1) were delivered into 2×10⁵ HEK293T cells by electroporation. Primers specific for DNA with base editing were designed and whether this qPCR assay yielded cycle threshold (Ct) values differing by ˜6, when comparing DNAs from nickase-transfected cells versus ABE-transfected cells was verified, to validate this approach. Twenty-four hours after treatment, qPCR detected on-target base editing in cells treated with 20 and 10 μg of ABE RNPs, but not in cells treated with lower amounts of ABE RNPs. NGS was performed to examine on-target base editing in cells treated with 20 and 10 μg ABE RNPs, and, 2.10%±0.22% (N=3) and 1.93%±0.53% (N=3) on-target base editing was observed, respectively (FIG. 1D). These were occurrences of target-specific base editing, since electroporation of ABE RNPs with a random sgRNA showed A to G changes at ABE site 1 in only 0.01% of samples. See, also Lyu et al. “Adenine Base Editor Ribnucleoproteins Delivered by Lentivirus-Like Particles Show High On-Target Base Editing and Undetectable RNA Off-Target Activities,” The CRISPR Journal 4(1): 69-81 (2021).

RNA off-target activities were examined at the USP38 hotspot. No off-target RNA editing was observed at the USP38 hotspot in any of the 6 samples, which was in sharp contrast to the high level (>15%) of RNA off-target editing with ABE plasmid DNA transfection (FIG. 1C, Table 4). The data indicate that ABE RNPs showed detectable on-target DNA editing, but undetectable off-target RNA editing 24 hours after delivery.

Although delivering ABE RNPs by electroporation greatly reduced RNA off-target activities, relatively low on-target base editing (<5%) occurred after electroporation of 20 μg (˜100 pmol) ABE RNPs, possibly due to ABE's relatively large protein size (˜1800 amino acid residues). It could be difficult to significantly improve on-target base editing efficiency simply by increasing the dosage. Thus, a more efficient ABE RNP delivery method is needed.

### Developing ABE RNP-Laden VLPs Via Packaging ABE RNPs in Lentiviral Capsids

Aptamer/ABP interactions can be used to package Cas9 RNPs into lentiviral capsids for efficient genome editing (Lyu et al., Nucleic Acids Res 2019, 47 (17): e99. Considering the different sizes of the proteins in question (1800 AA for ABE versus 1114 AA for SaCas9) and that the Cas9 proteins were from different species (Streptococcus pyogenes for ABE versus Staphylococcus aureus for SaCas9) and had different sgRNA scaffolds, three ways of sgRNA scaffold modification were used: 1) an MS2 aptamer replaced both the Tetraloop and the ST2 loop (FIG. 2A); 2) one copy of a com aptamer was used to replace the Tetraloop loop, and 3) one copy of com aptamer was used to replace the ST2 loop. The aptamer com was chosen since it was the most efficient aptamer in mediating SaCas9 RNP packaging into LV capsids. One copy of the aptamer was tested, since more than one copy greatly decreases RNA stability.

ABE-RNP was packaged into LV capsids by co-transfecting three plasmids into HEK293T cells: the envelope plasmid pMD2.G expressing the VSV-G protein, the target plasmid co-expressing ABE and various target-specific aptamer-modified sgRNAs, and the packaging plasmids modified by the corresponding ABPs (pspAX2-D64V-NC-MS2 for MS2 modified sgRNA and pspAX2-D64V-NC-com for com modified sgRNAs), as described recently. The supernatants containing capsid/ABE RNPs were used to transduce HEK293T cells. Then base editing activities with qPCR, were compared.

Single guide RNA sgRNA g1 and g5 were used to target ABE sites 1 and 5, respectively. These were the two sites previously shown to be successfully edited after transfecting the corresponding ABE expressing plasmid DNA (Gaudelli et al.). qPCR was used to detect the base editing activities of capsid/ABE RNPs, packaged with sgRNA containing 2×MS2, Tetra-com, and ST2-com, respectively. 20-160 times more edited products were detected in capsid/ABE RNP-treated cells than in negative control cells (ABE-g5 RNP treated cells as controls for ABE-g1 RNP-treated cells and vice versa), at ABE sites 1 and 5. All three types of ABE RNPs were functional (FIG. 2B, FIG. 2C).

For ABE sites 1 and 5, 2×MS2 modification showed the least base editing activity. For ABE site 5, the activities of single copy-com modified sgRNAs showed similar activities at the Tetraloop and ST2 loop locations. However, for ABE site 1, ST2-com modified RNPs performed significantly better than Tetra-com modified RNPs (P<0.0001). ST2-com modification of sgRNA was used for further experiments. The aptamer/ABP strategy was able to package and deliver functional ABE RNPs to human cells.

The base editing activity of the ABE RNP VLPs was examined by NGS. When targeting ABE site 1 in 2.5×104 HEK293T cells, 200 ng p24 of capsid-ABE RNPs generated A to G editing in 31.85% alleles (FIG. 3). When targeting ABE site 5 in 2.5×10⁴ HEK293T cells, 108 ng p24 of capsid-ABE RNPs (non-concentrated supernatant) generated A to G editing in 87.5% of all alleles (FIG. 2D). Whereas in cells treated with VLPs targeting ABE site 5, an A to G change was observed in 0.02% of alleles at ABE site 1, and in cells treated with VLPs targeting ABE site 1, an A to G change in 0.01% of alleles was observed at ABE site 5. These data show that the VLPs generated high-level site-specific base editing.

### Aptamer-Dependent Assembling of ABE RNP Laden VLPs

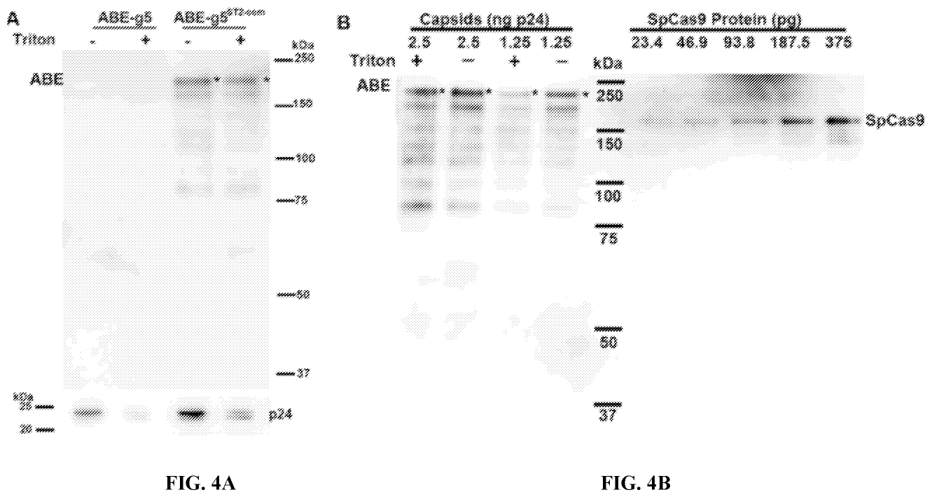

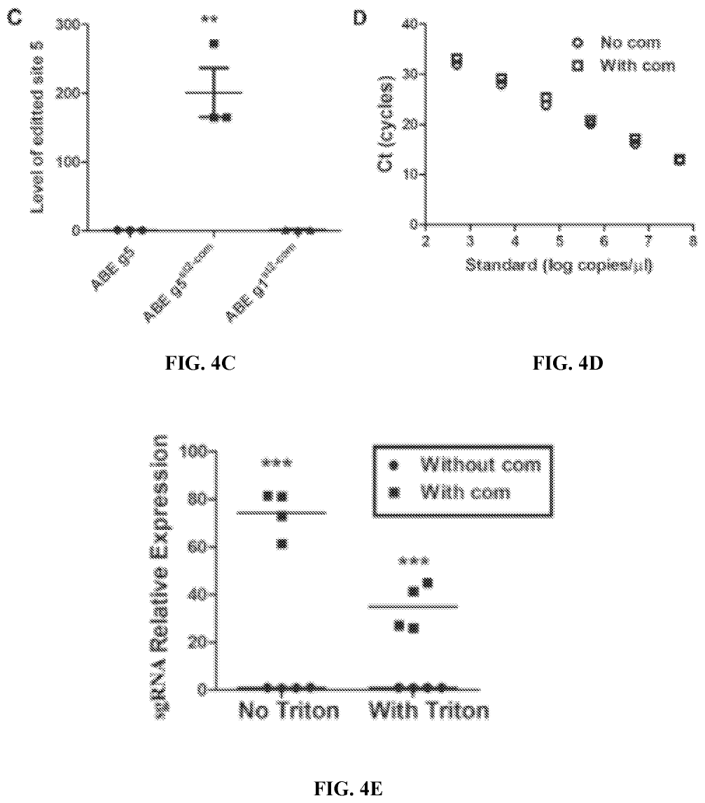

Whether aptamer/ABP interaction was necessary for the RNPs to be packaged inside the capsids as designed was analyzed. ABE protein content in capsids with ABE-g5 RNP (unmodified g5 sgRNA) and ABE-g5^{ST2-com} RNP (ST2-com modified g5 sgRNA) was compared. To eliminate possible ABE protein associated with vesicles or the particle membrane, we transiently treated the particles with 0.5% Triton™ X-100 buffer. This procedure reduced capsid protein p24 by over 100% (FIG. 4A).

ABE protein was then examined by Western blotting with an SpCas9 antibody. ABE was only detected in capsids with ABE-g5^{ST2-com} RNPs, but not in capsids with ABE-g5 RNPs (FIG. 4A). In addition, transient 0.5% Triton™ X-100 treatment decreased ABE amounts by 3050%. Compared with SpCas9 proteins of known concentration, the ABE amount in Triton-treated capsids was about 100 pg ABE/ng p24 (FIG. 4B, only considering the full-length ABE with an asterisk). Assuming 1.25×10⁷ capsids per ng p24, the ABE molecule numbers per capsid were estimated at 30 molecules per capsid.

Consistent with the lack of ABE protein in ABE-g5 RNP capsids, qPCR failed to detect base editing activities in cells treated with capsids packaged with ABE-g5 (without st2-com) RNPs (FIG. 4C). The data showed that ABE association with the capsids and base editing activities were aptamer-dependent.

sgRNA levels in the VLPs by RT-qPCR. qPCR was performed using known concentrations of the respective plasmid DNA (with or without com in sgRNA) to confirm that the com aptamer did not affect qPCR detection (FIG. 4D). In equal amounts (300 ng p24) of VLPs treated with and without Triton, the levels of g5^{ST2-com} sgRNA (with com) were 35.0±4.8 (N=4) and 74.2±4.8 (N=4) fold of those of g5 sgRNA (without com) respectively (FIG. 4E). The sgRNA qPCR data are consistent with our Western blotting data showing that com modification of sgRNA increased ABE levels in capsids and Triton X-100 treatment decreased it. Together, the data showed that packaging of ABE protein and sgRNA in the capsids and base editing activity of the VLPs all depended on com modification of sgRNA, confirming the role of ABP/aptamer interaction in packaging ABE RNPs.

### VLPs Enable Transient Expression of ABE RNPs in Human Cells

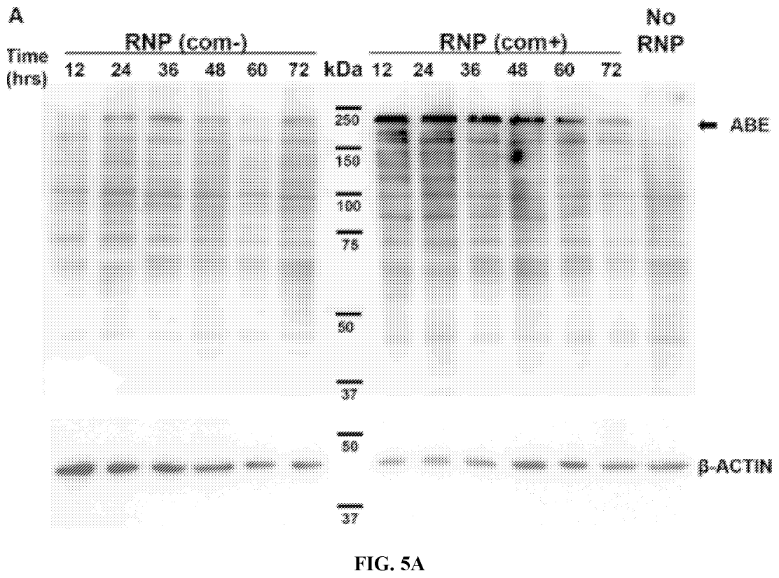

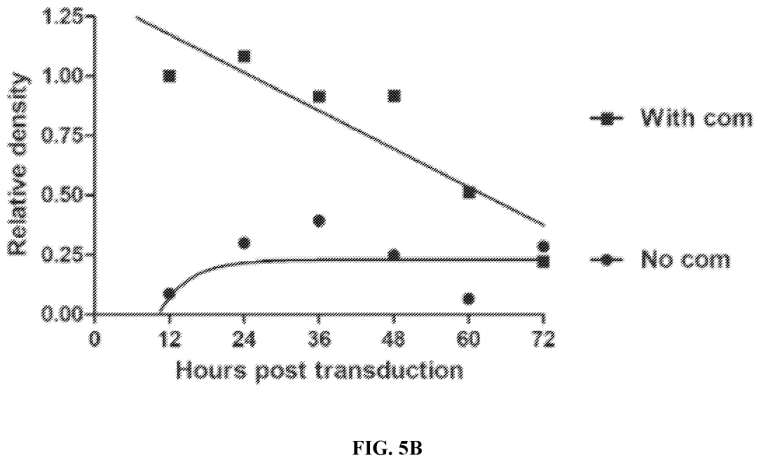

To determine the expression duration of ABE RNPs in human cells, transduced ABE-g5^{ST2-com} RNP-laden VLPs and ABE-g5 RNP-laden VLPs (each 100 ng p24/well) were transduced into HEK293T cells and ABE protein levels were measured every 12 hours. In RNP-treated but not control cells, Western blotting detected a band between 150 and 250 kDa (FIG. 5A), consistent with the expected size of ABE (204.7 kDa). In cells transduced with ABE-g5 RNP capsids, we observed a random fluctuation of low ABE levels (<25% of highest ABE-g5^{ST2-com} RNP level at all-time points). In cells transduced with ABE-g5^{ST2-com} RNP capsids, ABE levels were highest during the first 24 hours post-transduction and reduced slightly at 24-48 hours post transduction. At 48-72 hours post-transduction, ABE levels dropped to ˜25% of levels at 12 hours post-transduction, similar to levels in cells treated with ABE-g5 RNPs. At ˜60 hours post-transduction, ABE levels were half of those at 12 hours post-transduction (FIG. 5A, 5B).

In the experiment examining ABE in VLPs (FIG. 5A), ABE was not detected in ABE-g5 RNP VLPs. In that experiment, ABE-g5 RNP VLPs were subjected to an ultracentrifugation in a buffer without Triton™ X-100 and VLPs used to transduce cells were not centrifuged. It is likely that, the low background ABE in cells transduced with ABE-g5 RNP VLPs were the ABE in the capsid preparation. This was concentrated by the tangential low filtration system but not packaged in the capsids, and thus could be removed by ultracentrifugation. The data confirmed the short-term expression of ABE RNPs delivered by VLPs.

### ABE RNP-Laden VLPs Showed Undetectable Guide-Independent RNA Off-Target Activities

Whether ABE RNPs delivered by LV capsids generated detectable RNA off-targets was examined. ABE site 1 was targeted by ABE RNP-laden VLPs and plasmid DNA transfection. The conditions for the two delivery methods were determined, giving similar on-target base editing efficiencies. On-target and off-target activities were examined 24 hours after treatment, since that was the time point with the highest ABE level after VLP treatment. qPCR analysis of gDNA, 24 hours after treatment, revealed that transfection of 250 ng plasmid DNA showed similar gene editing activity on ABE site 1 as transducing 100 ng p24 of capsid-RNPs. NGS was performed on ABE site 1 genomic DNA and USP38 cDNA (amplified with F3 and R1 in FIG. 1A). ABE site 1 DNA had a slightly higher on-target A to G base editing rate in capsid-RNP transduced cells (14.5%) than in plasmid DNA-transfected cells (9.2%, Table 6).

TABLE 6
On-target base editing and RNA off-targets at the hotspot

| | DNA on-target editing | RNA off-target | |
|---|---|---|---|
| | (ABE site 1) | 1^{st} peak | 2^{nd} peak |
| No ABE (n = 1) | N/A | 0.02% | 0.09% |
| Capsid ABE RNP | 14.5% ± 0.9% | 0.025% ± 0.005% | 0.022% ± 0.007% |
| (n = 3) | | | |
| ABE DNA | 9.2% ± 0.8%* | 0.667% ± 0.133%* | 0.633% ± 0.145%* |
| transfection (n = 3) | | | |

*P < 0.05 when the values of capsid-RNP treated cells and DNA transfected cells were compared by t-tests.

RNA off-targets around the USP38 hotspot were analyzed. As a second peak was observed near the predicted hotspot in previous experiments (peak 2 in FIG. 1B), the percentages of A to G changes at both peaks were examined. In VLP-treated cells, A to G change rates, similar to negative control cells, were observed at both peaks, whereas in plasmid DNA transfected cells, significantly higher A to G change rates occurred at both peaks compared to VLP-treated cells (Table 5). In this experiment, DNA transfection resulted in ˜20 times lower RNA off-target rates than a previous DNA transfection experiment (0.667% versus ˜15% for the hotspot). The lower level of RNA off-targets in this experiment could have been caused by two non-exclusive mechanisms: 1) less DNA was transfected (250 ng versus 500 ng), and 2) RNA off-target activity was detected 24 hours rather than 48 hours after transfection. Nevertheless, delivering ABE RNPs by LV capsids did not result in detectable RNA off-targets, even though the on-target DNA base editing level was 56% higher than in cells treated with DNA transfection.

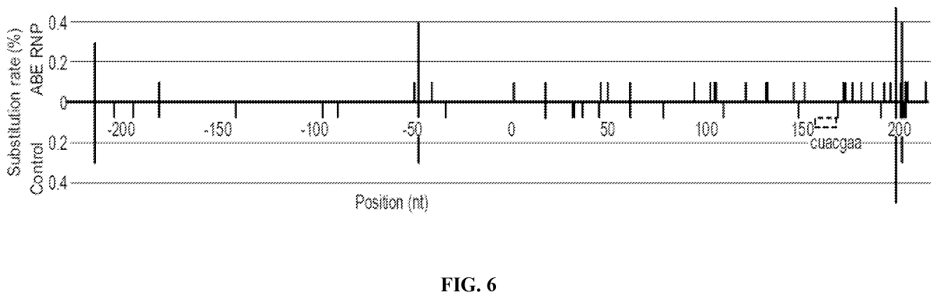

RNP off-target activities were examined 24 hours after VLP delivery because the ABE RNP expression duration data showed that ABE RNPs were highest 24 hours after transduction (FIG. 5A). RNA off-targets were also examined at 48 hours after VLP delivery and no RNA off-target activities were observed at the hotspot (FIG. 6). Since ABE protein levels decreased quickly after this time point, it is unlikely that further RNA off-target activities could be detected later. Thus, RNA off-target activity for ABE RNPs delivered by LV capsids was below the detection limit of the assay.

This work attempted to find an ABE delivery method with short activity duration, high base editing efficiency, and minimal RNA off-target activity. Two of the observations described above could help resolved the safety concerns caused by ABE's RNA off-target activities, especially for in vivo applications: 1) Delivering ABE RNPs generated detectable on-target DNA base editing with undetectable RNA off-target activities; and 2) Novel ABE RNP-laden VLPs, with high on-target DNA base editing efficiency and undetectable RNA off-target activity, were developed.

RNPs have been used in genome editing and cytosine base editing with improved specificity (Kim et al., Genome Res 2014, 24 (6): 1012-9). However, delivery of ABEs using RNPs has not been performed. As set forth above, delivery of ABE RNPs was performed by electroporation, and relatively low base editing activity (<5%) was observed when using ABE RNP amounts common to Cas9 RNP electroporation protocols. It is possible that using more ABE RNPs in electroporation may improve base editing activity. ABE RNP-laden VLPs were developed and packaged (˜30 ABE RNP molecules into each capsid particle). When targeting ABE site 1 in HEK293T cells, ABE RNP electroporation resulted in <5% base editing efficiency at 5 pg/cell (10 μg RNPs for 2×105 cells), whereas ABE RNP VLP transduction resulted in >30% base editing efficiency at 0.8 pg/cell (˜20 ng RNPs for 2.5×104 cells). When targeting the ABE g5 site, >85% base editing efficiency was obtained, at the dose of 0.43 pg/cell. Thus ABE RNP-laden VLPs resulted in much more efficient base editing, although much less ABE protein was used. This novel, ABE RNP-laden VLP is the first ABE RNP delivery vehicle demonstrating high base editing activity and low RNA off-target activity.

In addition to the high capsid assembly efficiency and base editing efficiency (>80% editing efficiency with unconcentrated VLPs), no RNA off-target activities were observed 24 hours after VLP delivery. RNA off-target generation before detection cannot be ruled out. However, typically, the earliest time to observe gene editing activity after delivering VLPs is about 16 hours post-transduction. Since escaping from the endosome system is a similar process to VLPs entering recipient cells, a comparable time should be needed for ABE RNPs to become functional after delivery. RNA off-targets, if any, could have been generated 16 to 24 hours after RNP delivery. This short time window could greatly reduce the chances of generating enough erroneous proteins to be harmful to the cells. Delivering ABE mRNA has reduced but still detectable RNA off-target activities (Gaudelli et al., Nat Biotechnol 2020 38 (7), 892-900), thus, delivering ABE RNP by VLPs is safer due to the undetectable RNA off-target activities.

Data provided herein show that VLP is an efficient ABE RNP delivery vehicle with minimal RNA off-target activity, without the need to use the ABE mutants with reduced RNA off-target activities. ABEs do not show detectable guide-independent DNA off-target activities. This development greatly reduces the safety risks caused by ABE's guide-independent RNA off-target activities, and enables efficient and safe delivery of ABE RNPs.

VLP-mediated ABE RNP delivery method delivers as little as 1/10 RNPs to each cell compared with current typical RNP electroporation protocols. This low amount of transiently expressed ABE RNPs delivered by VLPs should also achieve reduced guide-dependent DNA off-target activities.

In summary, ABE RNPs show guide-dependent DNA base editing but undetectable guide-independent RNA off-target activities. ABE RNPs can be efficiently and functionally packaged into lentiviral capsids. VLP-delivered ABE RNPs show high on-target DNA base editing activities and undetectable RNA off-target activities.

### Exemplary Embodiments

Embodiment 1. A mammalian expression plasmid comprising a eukaryotic promoter operably linked to a non-viral nucleic acid sequence, wherein the non-viral nucleic acid sequence comprises: (i) a nucleic acid sequence encoding an adenosine base pair editor (ABE), wherein the ABE is a fusion protein comprising an adenosine deaminase and a catalytically impaired CRISPR-associated endonuclease; and (ii) a guide RNA (gRNA) coding sequence, wherein the gRNA coding sequence comprises at least one aptamer coding sequence.

Embodiment 2. The mammalian expression plasmid of embodiment 1, wherein the catalytically impaired CRISPR-associated endonuclease coding sequence encodes a Cas9 D10A protein.

Embodiment 3. The mammalian expression plasmid of embodiment 1 or 2, wherein the adenine base editor is ABE 7.10 or ABE8.

Embodiment 4. The mammalian expression plasmid of any one of embodiments 1-3, wherein the at least one aptamer coding sequence encodes an aptamer sequence bound specifically by an ABP selected from the group consisting of MS2 coat protein, PP7 coat protein, lambda N RNA-binding domain, or Com protein.

Embodiment 5. The mammalian expression plasmid of any one of embodiments 1-4, wherein the aptamer is an MS2 aptamer sequence or a com aptamer sequence.

Embodiment 6. The mammalian expression plasmid of any one of embodiments 1-5 wherein the sgRNA coding sequence comprises at least one aptamer inserted into the tetraloop or the ST2 loop of the sgRNA coding sequence.

Embodiment 7. The mammalian expression plasmid of embodiment 6, wherein the sgRNA coding comprises at least one com aptamer inserted into the ST2 loop of the gRNA coding sequence.

Embodiment 8. A lentiviral packaging system comprising:
a) a packaging plasmid comprising a eukaryotic promoter operably linked to a Gag nucleotide sequence, wherein the Gag nucleotide sequence comprises a nucleocapsid (NC) coding sequence and a matrix protein (MA) coding sequence, wherein one or both of the NC coding sequence or the MA coding sequence comprises at least one non-viral aptamer-binding protein (ABP) nucleotide sequence, and wherein the packaging plasmid does not encode a functional integrase protein;
b) at least one mammalian expression plasmid of any one of claims 1-7; and
c) an envelope plasmid comprising an envelope glycoprotein coding sequence.

Embodiment 9. The lentiviral packaging system of embodiment 8, wherein the packaging plasmid further comprises a Rev nucleotide sequence and a Tat nucleotide sequence.

Embodiment 10. The lentiviral packaging system of embodiments 8 or 9, further comprising a second packaging plasmid comprising a Rev nucleotide sequence.

Embodiment 11. The lentiviral packaging system of any one of embodiments 8-10, wherein the at least one non-viral ABP nucleotide sequence encodes MS2 coat protein, PP7 coat protein, lambda N peptide, or Com protein.

Embodiment 12. A lentivirus-like particle comprising: a) a fusion protein comprising a nucleocapsid (NC) protein or a matrix (MA) protein wherein the NC protein or MA protein comprises at least one non-viral aptamer binding protein (ABP); and b) a ribonucleotide protein (RNP) complex comprising: (i) an adenine base editor (ABE), wherein the ABE is a fusion polypeptide comprising an adenine base editor and a catalytically impaired CRISPR-associated endonuclease; and (ii) a gRNA, wherein the lentivirus-like particle does not comprise a functional integrase protein.

Embodiment 13. The lentivirus-like particle of embodiment 12, wherein the catalytically impaired CRISPR-associated endonuclease is a catalytically impaired Cas9 protein, a catalytically impaired Cpf1 protein, or a derivative of either.

Embodiment 14. The lentivirus-like particle of embodiments 12 or 13, wherein the adenine base editor is ABE 7.10 or ABE 8.

Embodiment 15. A method of producing a lentivirus-like particle, the method comprising: a) transfecting a plurality of eukaryotic cells with the packaging plasmid, the at least one mammalian expression plasmid, and the envelope plasmid of the system of any one of claims 8-11; and b) culturing the transfected eukaryotic cells for sufficient time for lentivirus-like to be produced.

Embodiment 16. The method of embodiment 15, wherein the lentivirus-like particle comprises a ribonucleotide protein (RNP) complex comprising: (i) an adenine base editor (ABE), wherein the ABE is a fusion polypeptide comprising an adenosine deaminase and a catalytically impaired CRISPR-associated endonuclease; and (ii) a guide RNA.

Embodiment 17. The method of claim 16, wherein the plurality of eukaryotic cells are mammalian cells.

Embodiment 18. A lentivirus-like particle made by the method of any one of embodiments 15-17.

Embodiment 19. A method of modifying a genomic target sequence in a cell, the method comprising transducing a plurality of eukaryotic cells with a plurality of viral particles, wherein the plurality of viral particles comprise a lentivirus-like particle according embodiment 12, wherein the RNP binds to the genomic target sequence in genomic DNA of the cell and the ABE deaminates an adenine at the genomic target sequence, thereby modifying the genomic target sequence.

Embodiment 20. The method of embodiment 19, wherein the plurality of eukaryotic cells are mammalian cells.

Embodiment 21. The method of any one of embodiments 19 or 20, wherein the plurality of eukaryotic cells are cells present in subject.

Embodiment 22. The method of embodiment 21, wherein the subject is a human subject.

Embodiment 23. The method of embodiment 22, wherein the subject is injected with the plurality of viral particles.

Embodiment 24. A cell containing the plasmid of any one of embodiments 1-7.

Embodiment 25. A cell containing the lentiviral packaging system of any one of embodiments 8-11.

Embodiment 26. A cell containing the lentivirus-like particle of any one of embodiments 12-14.

Embodiment 27. A cell modified using the method of any one of embodiments 19-23.

Embodiment 28. A method for treating a disease in a subject comprising: a) obtaining cells from the subject; b) modifying the cells of the subject using the method of any one of embodiments 19-23; and c) administering the modified cells to the subject.

Embodiment 29. The method of embodiment 28, wherein the disease is cancer.

Embodiment 30. The method of embodiment 29, wherein the disease is sickle cell anemia.

Embodiment 31. The method of any one of embodiments 28-30, wherein the cells are T cells.

### Sequences

| | | |
|---|---|---|
| SEQ ID | MS2 coat protein | ATGGCTTCTAACTTTACTCAGTTCGTTCTCGTCGACAATGG |
| NO: 1 | (MCP) DNA | CGGAACTGGCGACGTGACTGTCGCCCCAAGCAACTTCGCT |
| | Sequence | AACGGGATCGCTGAATGGATCAGCTCTAACTCGCGTTCAC |
| | | AGGCTTACAAAGTAACCTGTAGCGTTCGTCAGAGCTCTGC |
| | | GCAGAATCGCAAATACACCATCAAAGTCGAGGTGCCTAAA |
| | | GGCGCCTGGCGTTCGTACTTAAATATGGAACTAACCATTC |
| | | CAATTTTCGCCACGAATTCCGACTGCGAGCTTATTGTTAAG |
| | | GCAATGCAAGGTCTCCTAAAAGATGGAAACCCGATTCCCT |
| | | CAGCAATCGCAGCAAACTCCGGCATCTAC |
| SEQ ID | MS2 coat protein | MASNFTQFVLVDNGGTGDVTVAPSNFANGIAEWISSNSRSQA |
| NO: 2 | (MCP) Amino | YKVTCSVRQSSAQNRKYTIKVEVPKGAWRSYLNMELTIPIFA |
| | Acid Sequence | TNSDCELIVKAMQGLLKDGNPIPSAIAANSGIY |
| SEQ ID | PP7 coat protein | tccaaaacaatagtcctctccgtaggggaggcaacacggactttgaccgaaatccagtcaaccg |
| NO: 3 | (PCP) DNA | ctgaccgacaaatctttgaagagaaagtagggcctcttgtgggccgactgcgcttgactgcaagc |
| | Sequence | ttgcgacaaaacggcgcaaagactgcctatagggtcaaccttaaactcgaccaagccgacgtgg |
| | | tcgatagcggtctccctaaggttcggtatacgcaggtctggagtcatgacgtaacaatcgtagcaa |
| | | acagcacagaagcctcccgaaaaagcctctacgatctgacgaaatccttggtggctacgtcaca |
| | | ggtggaagacctcgttgtcaaccttgtacctctgggtoga |
| SEQ ID | PP7 coat protein | SKTIVLSVGEATRTLTEIQSTADRQIFEEKVGPLVGRLRLTASL |
| NO: 3 | (PCP) Amino | RQNGAKTAYRVNLKLDQADVVDSGLPKVRYTQVWSHDVTI |
| | Acid Sequence | VANSTEASRKSLYDLTKSLVATSQVEDLVVNLVPLGR |
| SEQ ID | lambda N RNA- | ATGGATGCACAAACACGCCGCCGCGAACGTCGCGCAGAG |
| NO: 5 | binding domain | AAACAGGCTCAATGGAAAGCAGCAAAT |
| | (positions (1-22) | |
| | DNA Sequence | |
| SEQ ID | lambda N RNA- | MDAQTRRRERRAEKQAQWKAAN |
| NO: 6 | binding domain | |
| | (positions (1-22) | |
| | Amino Acid | |
| | Sequence | |
| SEQ ID | Com Protein DNA | atgaaatcaattcgctgtaaaaactgcaacaaactgttatttaaggcggattcctttgatcacattga |
| NO: 7 | Sequence | aatcaggtgtccgcgttgcaaacgtcacatcataatgctgaatgcctgcgagcatcccacggaga |
| | | aacattgtgggaaaagagaaaaaatcacgcattctgacgaaaccgtgcgttattgagtat |
| SEQ ID | Com Protein | MKSIRCKNCNKLLFKADSFDHIEIRCPRCKRHIIMLNACEHPT |
| NO: 8 | Amino Acid | EKHCGKREKITHSDETVRY |
| | Sequence | |
| | (GenBank | |
| | AAF01130.1) | |
| SEQ ID | MS2 aptamer | ACAUGAGGAUCACCCAUGU |
| NO: 9 | sequence (RNA) | |
| SEQ ID | MS2 aptamer | ACATGAGGATCACCCATGT |
| NO: 10 | sequence (DNA) | |
| SEQ ID | PP7 aptamer | GGAGCAGACGAUAUGGCGUCGCUCC |
| NO: 11 | sequence (RNA) | |
| SEQ ID | PP7 aptamer | GGAGCAGACGATATGGCGTCGCTCC |
| NO: 12 | sequence (DNA) | |
| SEQ ID | Box-B; lambda N | GGGCCCUGAAGAAGGGCCC |
| NO: 13 | RNA-binding | |
| | domain aptamer | |
| | sequence (RNA) | |
| SEQ ID | Box-B; lambda N | GGGCCCTGAAGAAGGGCCC |
| NO: 14 | RNA-binding | |
| | domain aptamer | |
| | sequence (DNA) | |
| SEQ ID | com aptamer RNA | CUGAAUGCCUGCGAGCAUC |
| NO: 15 | sequence | |
| SEQ ID | com aptamer DNA | CTGAATGCCTGCGAGCAT |
| NO: 16 | sequence | |
| SEQ ID | human beta | gctcgctttcttgctgtccaatttctattaaaggttcctttgttccctaagtccaactactaaactg |
| NO: 17 | hemoglobin | ggggatattatgaagggccttgagcatctggattctgcctaataaaaaacatttattttcattgc |
| | (HBB) 3′ UTR | |
| | (DNA) | |
| SEQ ID | human beta | gcucgcuuucuugcuguccaauuucuauuaaagguuccuuuguucccuaaguccaacu |
| NO: 18 | hemoglobin | acuaaacugggggauauuaugaagggccuugagcaucuggauucugccuaauaaaaaa |
| | (HBB) 3′ UTR | |
| | (RNA) | |
| SEQ ID | HIV-1 | ATACAGAAAGGCAATTTTAGGAACCAAAGAAAGACTGTTA |
| NO: 19 | Nucleocapsid | AGTGTTTCAATTGTGGCAAAGAAGGGCACATAGCCAAAAA |
| | (NC) DNA | TTGCAGGGCCCCTAGGAAAAAGGGCTGTTGGAAATGTGGA |
| | Sequence | AAGGAAGGACACCAAATGAAAGATTGTACTGAGAGACAG |
| | | GCTAAT |
| SEQ ID | HIV-1 | IQKGNFRNQRKTVKCFNCGKEGHIAKNCRAPRKKGCWKCG |
| NO: 20 | Nucleocapsid | KEGHQMKDCTERQAN |
| | (NC) Amino Acid | |
| | Sequence | |
| SEQ ID | HIV-1 Matrix | atgggtgcgagagcgtcagtattaagcgggggagaattagatcgatgggaaaaaattcggttaa |
| NO: 21 | protein (MA) | ggccagggggaaagaaaaaatataaattaaaacatatagtatgggcaagcagggagctagaac |
| | DNA Sequence | gattcgcagttaatcctggcctgttagaaacatcagaaggctgtagacaaatactgggacagctac |
| | | aaccatcccttcagacaggatcagaagaacttagatcattatataatacagtagcaaccctctattgt |
| | | gtgcatcaaaggatagagataaaagacaccaaggaagctttagacaagatagaggaagagcaa |
| | | aacaaaagtaagaaaaaagcacagcaagcagcagctgacacaggacacagcaatcaggtcag |
| | | ccaaaattac |
| SEQ ID | HIV-1 Matrix | GARASVLSGGELDRWEKIRLRPGGKKKYKLKHIVWASRELE |
| NO: 22 | protein (MA) | RFAVNPGLLETSEGCRQILGQLQPSLQTGSEELRSLYNTVATL |
| | Amino Acid | YCVHQRIEIKDTKEALDKIEEEQNKSKKKAQQAAADTGHSN |
| | Sequence | QVSQNY |
| SEQ ID | HIV-1 Viral | ATGGAACAAGCCCCAGAAGACCAGGGACCGCAGAGGGAA |
| NO: 23 | Protein (VPR) | CCATACAATGAATGGACACTAGAACTTTTAGAGGAACTCA |
| | DNA Sequence | AGCGGGAAGCAGTCAGACACTTTCCTAGACCATGGCTTCA |
| | | TGGCTTAGGACAACATATCTATGAAACCTATGGAGATACT |
| | | TGGACGGGGGTGGAAGCTATAATAAGAATTCTGCAACGAC |
| | | TACTGTTTGTCCATTTCAGAATTGGGTGCCAGCATAGCCGA |
| | | ATAGGCATTCTAAGACAGAGAAGAGCAAGAAATGGAGCC |
| | | AGTAGATCCTAA |
| SEQ ID | HIV-1 Viral | MEQAPEDQGPQREPYNEWTLELLEELKREAVRHFPRPWLHG |
| NO: 24 | Protein (VPR) | LGQHIYETYGDTWTGVEAIIRILORLLFVHFRIGCQHSRIGILR |
| | Amino Acid | QRRARNGASRS |
| | Sequence | |
| SEQ ID | HIV-1 Negative | atgggtTgcaagtggtcaaaaagtagtgtgattggatggcctgctgtaagggaaagaatgagac |
| NO: 25 | Regulatory Factor | gagctgagccagcagcagatggggtgggagcagtatctcgagacctagaaaaacatggagca |
| | (NEF) DNA | atcacaagtagcaatacagcagctaacaatgctgcttgtgcctggctagaagcacaagaggagg |
| | Sequence with | aagaggtgggttttccagtcacacctcaggtacctttaagaccaatgacttacaaggcagctgtag |
| | codon changes to | atcttagccactttttaaaagaaaaggggggactggaagggctaattcactcccaaagaagacaa |
| | enhance | gatatccttgatctgtggatctaccacacacaaggctacttccctgattggcagaactacacacca |
| | packaging in the | gggccaggggtcagatatccactgacctttggatggtgctacaagctagtaccagttgagccaga |
| | virus core (G3C, | taagCtGgaagaggccaataaaggagagaacaccagcttgttacaccctgtgagcctgcatgg |
| | V153L, and | aatggatgaccctgGAagagaagtgttagagtggaggtttgacagccgcctagcatttcatcac |
| | E177G mutations; | gtggcccgagagctgcatccggagtacttcaagaactgc (The yellow positions are |
| | underlined) | changed to code for the changes explained in seq ID. 8. |
| SEQ ID | HIV-1 Negative | MGCKWSKSSVIGWPAVRERMRRAEPAADGVGAVSRDLEKH |
| NO: 26 | Regulatory Factor | GAITSSNTAANNAACAWLEAQEEEEVGFPVTPQVPLRPMTY |
| | (NEF) Amino | KAAVDLSHFLKEKGGLEGLIHSQRRQDILDLWIYHTQGYFPD |
| | Acid Sequence | WQNYTPGPGVRYPLTFGWCYKLVPVEPDKLEEANKGENTSL |
| | with mutation to | LHPVSLHGMDDPGREVLEWRFDSRLAFHHVARELHPEYFKN |
| | enhance | C |
| | packaging in the | |
| | virus core (G3C, | |
| | V153L, and | |
| | E177G mutations; | |
| | underlined) | |
| SEQ ID | Nucleic acid | atgaaacggacagccgacggaagcgagttcgagtcaccaaagaagaagcggaaagtctctga |
| NO: 27 | encoding ABE- | agtcgagtttagccacgagtattggatgaggcacgcactgaccctggcaaagcgagcatgggat |
| | SpCas9(D10A) | gaaagagaagtccccgtgggcgccgtgctggtgcacaacaatagagtgatcggagagggatg |
| | fusion protein | gaacaggccaatcggccgccacgaccctaccgcacacgcagagatcatggcactgaggcagg |
| | | gaggcctggtcatgcagaattaccgcctgatcgatgccaccctgtatgtgacactggagccatgc |
| | | gtgatgtgcgcaggagcaatgatccacagcaggatcggaagagtggtgttcggagcacgggac |
| | | gccaagaccggcgcagcaggctccctgatggatgtgctgcaccaccccggcatgaaccaccg |
| | | ggtggagatcacagagggaatcctggcagacgagtgcgccgccctgctgagcgatttctttaga |
| | | atgcggagacaggagatcaaggcccagaagaaggcacagagctccaccgactctggaggatc |
| | | tagcggaggatcctctggaagcgagacaccaggcacaagcgagtccgccacaccagagagct |
| | | ccggcggctcctccggaggatcctctgaggtggagttttcccacgagtactggatgagacatgcc |
| | | ctgaccctggccaagagggcacgcgatgagagggaggtgcctgtgggagccgtgctggtgct |
| | | gaacaatagagtgatcggcgagggctggaacagagccatcggcctgcacgacccaacagccc |
| | | atgccgaaattatggccctgagacagggcggcctggtcatgcagaactacagactgattgacgc |
| | | caccctgtacgtgacattcgagccttgcgtgatgtgcgccggcgccatgatccactctaggatog |
| | | gccgcgtggtgtttggcgtgaggaacgcaaaaaccggcgccgcaggctccctgatggacgtgc |
| | | tgcactaccccggcatgaatcaccgcgtcgaaattaccgagggaatcctggcagatgaatgtgc |
| | | cgccctgctgtgctatttctttoggatgcctagacaggtgttcaatgctcagaagaaggcccagag |
| | | ctccaccgactccggaggatctagcggaggctcctctggctctgagacacctggcacaagcga |
| | | gagcgcaacacctgaaagcagcgggggcagcagcggggggtcagacaagaagtacagcatc |
| | | ggcctggccatcggcaccaactctgtgggctgggccgtgatcaccgacgagtacaaggtgccc |
| | | agcaagaaattcaaggtgctgggcaacaccgaccggcacagcatcaagaagaacctgatcgg |
| | | agccctgctgttcgacagcggcgaaacagccgaggccacccggctgaagagaaccgccaga |
| | | agaagatacaccagacggaagaaccggatctgctatctgcaagaGATCTTCAGCAA |
| | | CGAGATGGCCAAGGTGGACGACAGCTTCTTCCACAGACTG |
| | | GAAGAGTCCTTCCTGGTGGAAGAGGATAAGAAGCACGAG |
| | | CGGCACCCCATCTTCGGCAACATCGTGGACGAGGTGGCCT |
| | | ACCACGAGAAGTACCCCACCATCTACCACCTGAGAAAGAA |
| | | ACTGGTGGACAGCACCGACAAGGCCGACCTGCGGCTGATC |
| | | TATCTGGCCCTGGCCCACATGATCAAGTTCCGGGGCCACTT |
| | | CCTGATCGAGGGCGACCTGAACCCCGACAACAGCGACGTG |
| | | GACAAGCTGTTCATCCAGCTGGTGCAGACCTACAACCAGC |
| | | TGTTCGAGGAAAACCCCATCAACGCCAGCGGCGTGGACGC |
| | | CAAGGCCATCCTGTCTGCCAGACTGAGCAAGAGCAGACGG |
| | | CTGGAAAATCTGATCGCCCAGCTGCCCGGCGAGAAGAAGA |
| | | ATGGCCTGTTCGGAAACCTGATTGCCCTGAGCCTGGGCCT |
| | | GACCCCCAACTTCAAGAGCAACTTCGACCTGGCCGAGGAT |
| | | GCCAAACTGCAGCTGAGCAAGGACACCTACGACGACGAC |
| | | CTGGACAACCTGCTGGCCCAGATCGGCGACCAGTACGCCG |
| | | ACCTGTTTCTGGCCGCCAAGAACCTGTCCGACGCCATCCTG |
| | | CTGAGCGACATCCTGAGAGTGAACACCGAGATCACCAAGG |
| | | CCCCCCTGAGCGCCTCTATGATCAAGAGATACGACGAGCA |
| | | CCACCAGGACCTGACCCTGCTGAAAGCTCTCGTGCGGCAG |
| | | CAGCTGCCTGAGAAGTACAAAGAGATTTTCTTCGACCAGA |
| | | GCAAGAACGGCTACGCCGGCTACATTGACGGCGGAGCCA |
| | | GCCAGGAAGAGTTCTACAAGTTCATCAAGCCCATCCTGGA |
| | | AAAGATGGACGGCACCGAGGAACTGCTCGTGAAGCTGAA |
| | | CAGAGAGGACCTGCTGCGGAAGCAGCGGACCTTCGACAA |
| | | CGGCAGCATCCCCCACCAGATCCACCTGGGAGAGCTGCAC |
| | | GCCATTCTGCGGCGGCAGGAAGATTTTTACCCATTCCTGA |
| | | AGGACAACCGGGAAAAGATCGAGAAGATCCTGACCTTCC |
| | | GCATCCCCTACTACGTGGGCCCTCTGGCCAGGGGAAACAG |
| | | CAGATTCGCCTGGATGACCAGAAAGAGCGAGGAAACCAT |
| | | CACCCCCTGGAACTTCGAGGAAGTGGTGGACAAGGGCGCT |
| | | TCCGCCCAGAGCTTCATCGAGCGGATGACCAACTTCGATA |
| | | AGAACCTGCCCAACGAGAAGGTGCTGCCCAAGCACAGCCT |
| | | GCTGTACGAGTACTTCACCGTGTATAACGAGCTGACCAAA |
| | | GTGAAATACGTGACCGAGGGAATGAGAAAGCCCGCCTTCC |
| | | TGAGCGGCGAGCAGAAAAAGGCCATCGTGGACCTGCTGTT |
| | | CAAGACCAACCGGAAAGTGACCGTGAAGCAGCTGAAAGA |
| | | GGACTACTTCAAGAAAATCGAGTGCTTCGACTCCGTGGAA |
| | | ATCTCCGGCGTGGAAGATCGGTTCAACGCCTCCCTGGGCA |
| | | CATACCACGATCTGCTGAAAATTATCAAGGACAAGGACTT |
| | | CCTGGACAATGAGGAAAACGAGGACATTCTGGAAGATATC |
| | | GTGCTGACCCTGACACTGTTTGAGGACAGAGAGATGATCG |
| | | AGGAACGGCTGAAAACCTATGCCCACCTGTTCGACGACAA |
| | | AGTGATGAAGCAGCTGAAGCGGCGGAGATACACCGGCTG |
| | | GGGCAGGCTGAGCCGGAAGCTGATCAACGGCATCCGGGA |
| | | CAAGCAGTCCGGCAAGACAATCCTGGATTTCCTGAAGTCC |
| | | GACGGCTTCGCCAACAGAAACTTCATGCAGCTGATCCACG |
| | | ACGACAGCCTGACCTTTAAAGAGGACATCCAGAAAGCCCA |
| | | GGTGTCCGGCCAGGGCGATAGCCTGCACGAGCACATTGCC |
| | | AATCTGGCCGGCAGCCCCGCCATTAAGAAGGGCATCCTGC |
| | | AGACAGTGAAGGTGGTGGACGAGCTCGTGAAAGTGATGG |
| | | GCCGGCACAAGCCCGAGAACATCGTGATCGAAATGGCCA |
| | | GAGAGAACCAGACCACCCAGAAGGGACAGAAGAACAGCC |
| | | GCGAGAGAATGAAGCGGATCGAAGAGGGCATCAAAGAGC |
| | | TGGGCAGCCAGATCCTGAAAGAACACCCCGTGGAAAACA |
| | | CCCAGCTGCAGAACGAGAAGCTGTACCTGTACTACCTGCA |
| | | GAATGGGCGGGATATGTACGTGGACCAGGAACTGGACATC |
| | | AACCGGCTGTCCGACTACGATGTGGACCATATCGTGCCTC |
| | | AGAGCTTTCTGAAGGACGACTCCATCGACAACAAGGTGCT |
| | | GACCAGAAGCGACAAGAACCGGGGCAAGAGCGACAACGT |
| | | GCCCTCCGAAGAGGTCGTGAAGAAGATGAAGAACTACTG |
| | | GCGGCAGCTGCTGAACGCCAAGCTGATTACCCAGAGAAAG |
| | | TTCGACAATCTGACCAAGGCCGAGAGAGGCGGCCTGAGCG |
| | | AACTGGATAAGGCCGGCTTCATCAAGAGACAGCTGGTGGA |
| | | AACCCGGCAGATCACAAAGCACGTGGCACAGATCCTGGAC |
| | | TCCCGGATGAACACTAAGTACGACGAGAATGACAAGCTGA |
| | | TCCGGGAAGTGAAAGTGATCACCCTGAAGTCCAAGCTGGT |
| | | GTCCGATTTCCGGAAGGATTTCCAGTTTTACAAAGTGCGC |
| | | GAGATCAACAACTACCACCACGCCCACGACGCCTACCTGA |
| | | ACGCCGTCGTGGGAACCGCCCTGATCAAAAAGTACCCTAA |
| | | GCTGGAAAGCGAGTTCGTGTACGGCGACTACAAGGTGTAC |
| | | GACGTGCGGAAGATGATCGCCAAGAGCGAGCAGGAAATC |
| | | GGCAAGGCTACCGCCAAGTACTTCTTCTACAGCAACATCA |
| | | TGAACTTTTTCAAGACCGAGATTACCCTGGCCAACGGCGA |
| | | GATCCGGAAGCGGCCTCTGATCGAGACAAACGGCGAAAC |
| | | CGGGGAGATCGTGTGGGATAAGGGCCGGGATTTTGCCACC |
| | | GTGCGGAAAGTGCTGAGCATGCCCCAAGTGAATATCGTGA |
| | | AAAAGACCGAGGTGCAGACAGGCGGCTTCAGCAAAGAGT |
| | | CTATCCTGCCCAAGAGGAACAGCGATAAGCTGATCGCCAG |
| | | AAAGAAGGACTGGGACCCTAAGAAGTACGGCGGCTTCGA |
| | | CAGCCCCACCGTGGCCTATTCTGTGCTGGTGGTGGCCAAA |
| | | GTGGAAAAGGGCAAGTCCAAGAAACTGAAGAGTGTGAAA |
| | | GAGCTGCTGGGGATCACCATCATGGAAAGAAGCAGCTTCG |
| | | AGAAGAATCCCATCGACTTTCTGGAAGCCAAGGGCTACAA |
| | | AGAAGTGAAAAAGGACCTGATCATCAAGCTGCCTAAGTAC |
| | | TCCCTGTTCGAGCTGGAAAACGGCCGGAAGAGAATGCTGG |
| | | CCTCTGCCGGCGAACTGCAGAAGGGAAACGAACTGGCCCT |
| | | GCCCTCCAAATATGTGAACTTCCTGTACCTGGCCAGCCACT |
| | | ATGAGAAGCTGAAGGGCTCCCCCGAGGATAATGAGCAGA |
| | | AACAGCTGTTTGTGGAACAGCACAAGCACTACCTGGACGA |
| | | GATCATCGAGCAGATCAGCGAGTTCTCCAAGAGAGTGATC |
| | | CTGGCCGACGCTAATCTGGACAAAGTGCTGTCCGCCTACA |
| | | ACAAGCACCGGGATAAGCCCATCAGAGAGCAGGCCGAGA |
| | | ATATCATCCACCTGTTTACCCTGACCAATCTGGGAGCCCCT |
| | | GCCGCCTTCAAGTACTTTGACACCACCATCGACCGGAAGA |
| | | GGTACACCAGCACCAAAGAGGTGCTGGACGCCACCCTGAT |
| | | CCACCAGAGCATCACCGGCCTGTACGAGACACGGATCGAC |
| | | CTGTCTCAGCTGGGAGGCGACAAAAGGCCGGCGGCCACG |
| | | AAAAAGGCCGGccaggcaaaaaagaaaaagggatcctaa |
| SEQ ID | ABEMAX fusion | MKRTADGSEFESPKKKRKVSEVEFSHEYWMRHALTLAKRA |
| NO: 28 | protein comprising | WDEREVPVGAVLVHNNRVIGEGWNRPIGRHDPTAHAEIMAL |
| | deaminase and | RQGGLVMQNYRLIDATLYVTLEPCVMCAGAMIHSRIGRVVF |
| | spCas9 (D10A) | GARDAKTGAAGSLMDVLHHPGMNHRVEITEGILADECAALL |
| | | SDFFRMRRQEIKAQKKAQSSTDSGGSSGGSSGSETPGTSESAT |
| | | PESSGGSSGGSSEVEFSHEYWMRHALTLAKRARDEREVPVG |
| | | AVLVLNNRVIGEGWNRAIGLHDPTAHAEIMALRQGGLVMQ |
| | | NYRLIDATLYVTFEPCVMCAGAMIHSRIGRVVFGVRNAKTG |
| | | AAGSLMDVLHYPGMNHRVEITEGILADECAALLCYFFRMPR |
| | | QVFNAQKKAQSSTDSGGSSGGSSGSETPGTSESATPESSGGSS |
| | | GGSDKKYSIGLAIGTNSVGWAVITDEYKVPSKKFKVLGNTDR |
| | | HSIKKNLIGALLFDSGETAEATRLKRTARRRYTRRKNRICYLQ |
| | | EIFSNEMAKVDDSFFHRLEESFLVEEDKKHERHPIFGNIVDEV |
| | | AYHEKYPTIYHLRKKLVDSTDKADLRLIYLALAHMIKFRGHF |
| | | LIEGDLNPDNSDVDKLFIQLVQTYNQLFEENPINASGVDAKAI |
| | | LSARLSKSRRLENLIAQLPGEKKNGLFGNLIALSLGLTPNFKS |
| | | NFDLAEDAKLQLSKDTYDDDLDNLLAQIGDQYADLFLAAKN |
| | | LSDAILLSDILRVNTEITKAPLSASMIKRYDEHHQDLTLLKAL |
| | | VRQQLPEKYKEIFFDQSKNGYAGYIDGGASQEEFYKFIKPILE |
| | | KMDGTEELLVKLNREDLLRKQRTFDNGSIPHQIHLGELHAIL |
| | | RRQEDFYPFLKDNREKIEKILTFRIPYYVGPLARGNSRFAWMT |
| | | RKSEETITPWNFEEVVDKGASAQSFIERMTNFDKNLPNEKVL |
| | | PKHSLLYEYFTVYNELTKVKYVTEGMRKPAFLSGEQKKAIV |
| | | DLLFKTNRKVTVKQLKEDYFKKIECFDSVEISGVEDRFNASL |
| | | GTYHDLLKIIKDKDFLDNEENEDILEDIVLTLTLFEDREMIEER |
| | | LKTYAHLFDDKVMKQLKRRRYTGWGRLSRKLINGIRDKQSG |
| | | KTILDFLKSDGFANRNFMQLIHDDSLTFKEDIQKAQVSGQGD |
| | | SLHEHIANLAGSPAIKKGILQTVKVVDELVKVMGRHKPENIVI |
| | | EMARENQTTQKGQKNSRERMKRIEEGIKELGSQILKEHPVEN |
| | | TQLQNEKLYLYYLQNGRDMYVDQELDINRLSDYDVDHIVPQ |
| | | SFLKDDSIDNKVLTRSDKNRGKSDNVPSEEVVKKMKNYWRQ |
| | | LLNAKLITQRKFDNLTKAERGGLSELDKAGFIKRQLVETRQIT |
| | | KHVAQILDSRMNTKYDENDKLIREVKVITLKSKLVSDFRKDF |
| | | QFYKVREINNYHHAHDAYLNAVVGTALIKKYPKLESEFVYG |
| | | DYKVYDVRKMIAKSEQEIGKATAKYFFYSNIMNFFKTEITLA |
| | | NGEIRKRPLIETNGETGEIVWDKGRDFATVRKVLSMPQVNIV |
| | | KKTEVQTGGFSKESILPKRNSDKLIARKKDWDPKKYGGFDSP |
| | | TVAYSVLVVAKVEKGKSKKLKSVKELLGITIMERSSFEKNPID |
| | | FLEAKGYKEVKKDLIIKLPKYSLFELENGRKRMLASAGELQK |
| | | GNELALPSKYVNFLYLASHYEKLKGSPEDNEQKQLFVEQHK |
| | | HYLDEIIEQISEFSKRVILADANLDKVLSAYNKHRDKPIREQA |
| | | ENIIHLFTLTNLGAPAAFKYFDTTIDRKRYTSTKEVLDATLIHQ |
| | | SITGLYETRIDLSQLGGDKRPAATKKAGQAKKKKGS* |
| | | ATGGACTATAAGGACCACGACGGAGACTACAAGGATCAT |
| | | GATATTGATTACAAAGACGATGACGATAAGATGGCCCCAA |
| | | AGAAGAAGCGGAAGGTCGGTATCCACGGAGTCCCAGCAG |
| | | CCGACAAGAAGTACAGCATCGGCCTGGACATCGGCACCAA |
| SEQ ID | Nucleic acid | CTCTGTGGGCTGGGCCGTGATCACCGACGAGTACAAGGTG |
| NO: 29 | sequence encoding | CCCAGCAAGAAATTCAAGGTGCTGGGCAACACCGACCGGC |
| | spCas9 (D10A) | ACAGCATCAAGAAGAACCTGATCGGAGCCCTGCTGTTCGA |
| | | CAGCGGCGAAACAGCCGAGGCCACCCGGCTGAAGAGAAC |
| | | CGCCAGAAGAAGATACACCAGACGGAAGAACCGGATCTG |
| | | CTATCTGCAAGAGATCTTCAGCAACGAGATGGCCAAGGTG |
| | | GACGACAGCTTCTTCCACAGACTGGAAGAGTCCTTCCTGG |
| | | TGGAAGAGGATAAGAAGCACGAGCGGCACCCCATCTTCG |
| | | GCAACATCGTGGACGAGGTGGCCTACCACGAGAAGTACCC |
| | | CACCATCTACCACCTGAGAAAGAAACTGGTGGACAGCACC |
| | | GACAAGGCCGACCTGCGGCTGATCTATCTGGCCCTGGCCC |
| | | ACATGATCAAGTTCCGGGGCCACTTCCTGATCGAGGGCGA |
| | | CCTGAACCCCGACAACAGCGACGTGGACAAGCTGTTCATC |
| | | CAGCTGGTGCAGACCTACAACCAGCTGTTCGAGGAAAACC |
| | | CCATCAACGCCAGCGGCGTGGACGCCAAGGCCATCCTGTC |
| | | TGCCAGACTGAGCAAGAGCAGACGGCTGGAAAATCTGATC |
| | | GCCCAGCTGCCCGGCGAGAAGAAGAATGGCCTGTTCGGAA |
| | | ACCTGATTGCCCTGAGCCTGGGCCTGACCCCCAACTTCAA |
| | | GAGCAACTTCGACCTGGCCGAGGATGCCAAACTGCAGCTG |
| | | AGCAAGGACACCTACGACGACGACCTGGACAACCTGCTGG |
| | | CCCAGATCGGCGACCAGTACGCCGACCTGTTTCTGGCCGC |
| | | CAAGAACCTGTCCGACGCCATCCTGCTGAGCGACATCCTG |
| | | AGAGTGAACACCGAGATCACCAAGGCCCCCCTGAGCGCCT |
| | | CTATGATCAAGAGATACGACGAGCACCACCAGGACCTGAC |
| | | CCTGCTGAAAGCTCTCGTGCGGCAGCAGCTGCCTGAGAAG |
| | | TACAAAGAGATTTTCTTCGACCAGAGCAAGAACGGCTACG |
| | | CCGGCTACATTGACGGCGGAGCCAGCCAGGAAGAGTTCTA |
| | | CAAGTTCATCAAGCCCATCCTGGAAAAGATGGACGGCACC |
| | | GAGGAACTGCTCGTGAAGCTGAACAGAGAGGACCTGCTGC |
| | | GGAAGCAGCGGACCTTCGACAACGGCAGCATCCCCCACCA |
| | | GATCCACCTGGGAGAGCTGCACGCCATTCTGCGGCGGCAG |
| | | GAAGATTTTTACCCATTCCTGAAGGACAACCGGGAAAAGA |
| | | TCGAGAAGATCCTGACCTTCCGCATCCCCTACTACGTGGG |
| | | CCCTCTGGCCAGGGGAAACAGCAGATTCGCCTGGATGACC |
| | | AGAAAGAGCGAGGAAACCATCACCCCCTGGAACTTCGAG |
| | | GAAGTGGTGGACAAGGGCGCTTCCGCCCAGAGCTTCATCG |
| | | AGCGGATGACCAACTTCGATAAGAACCTGCCCAACGAGAA |
| | | GGTGCTGCCCAAGCACAGCCTGCTGTACGAGTACTTCACC |
| | | GTGTATAACGAGCTGACCAAAGTGAAATACGTGACCGAGG |
| | | GAATGAGAAAGCCCGCCTTCCTGAGCGGCGAGCAGAAAA |
| | | AGGCCATCGTGGACCTGCTGTTCAAGACCAACCGGAAAGT |
| | | GACCGTGAAGCAGCTGAAAGAGGACTACTTCAAGAAAAT |
| | | CGAGTGCTTCGACTCCGTGGAAATCTCCGGCGTGGAAGAT |
| | | CGGTTCAACGCCTCCCTGGGCACATACCACGATCTGCTGA |
| | | AAATTATCAAGGACAAGGACTTCCTGGACAATGAGGAAA |
| | | ACGAGGACATTCTGGAAGATATCGTGCTGACCCTGACACT |
| | | GTTTGAGGACAGAGAGATGATCGAGGAACGGCTGAAAAC |
| | | CTATGCCCACCTGTTCGACGACAAAGTGATGAAGCAGCTG |
| | | AAGCGGCGGAGATACACCGGCTGGGGCAGGCTGAGCCGG |
| | | AAGCTGATCAACGGCATCCGGGACAAGCAGTCCGGCAAG |
| | | ACAATCCTGGATTTCCTGAAGTCCGACGGCTTCGCCAACA |
| | | GAAACTTCATGCAGCTGATCCACGACGACAGCCTGACCTT |
| | | TAAAGAGGACATCCAGAAAGCCCAGGTGTCCGGCCAGGG |
| | | CGATAGCCTGCACGAGCACATTGCCAATCTGGCCGGCAGC |
| | | CCCGCCATTAAGAAGGGCATCCTGCAGACAGTGAAGGTGG |
| | | TGGACGAGCTCGTGAAAGTGATGGGCCGGCACAAGCCCG |
| | | AGAACATCGTGATCGAAATGGCCAGAGAGAACCAGACCA |
| | | CCCAGAAGGGACAGAAGAACAGCCGCGAGAGAATGAAGC |
| | | GGATCGAAGAGGGCATCAAAGAGCTGGGCAGCCAGATCC |
| | | TGAAAGAACACCCCGTGGAAAACACCCAGCTGCAGAACG |
| | | AGAAGCTGTACCTGTACTACCTGCAGAATGGGCGGGATAT |
| | | GTACGTGGACCAGGAACTGGACATCAACCGGCTGTCCGAC |
| | | TACGATGTGGACCATATCGTGCCTCAGAGCTTTCTGAAGG |
| | | ACGACTCCATCGACAACAAGGTGCTGACCAGAAGCGACA |
| | | AGAACCGGGGCAAGAGCGACAACGTGCCCTCCGAAGAGG |
| | | TCGTGAAGAAGATGAAGAACTACTGGCGGCAGCTGCTGAA |
| | | CGCCAAGCTGATTACCCAGAGAAAGTTCGACAATCTGACC |
| | | AAGGCCGAGAGAGGCGGCCTGAGCGAACTGGATAAGGCC |
| | | GGCTTCATCAAGAGACAGCTGGTGGAAACCCGGCAGATCA |
| | | CAAAGCACGTGGCACAGATCCTGGACTCCCGGATGAACAC |
| | | TAAGTACGACGAGAATGACAAGCTGATCCGGGAAGTGAA |
| | | AGTGATCACCCTGAAGTCCAAGCTGGTGTCCGATTTCCGG |
| | | AAGGATTTCCAGTTTTACAAAGTGCGCGAGATCAACAACT |
| | | ACCACCACGCCCACGACGCCTACCTGAACGCCGTCGTGGG |
| | | AACCGCCCTGATCAAAAAGTACCCTAAGCTGGAAAGCGAG |
| | | TTCGTGTACGGCGACTACAAGGTGTACGACGTGCGGAAGA |
| | | TGATCGCCAAGAGCGAGCAGGAAATCGGCAAGGCTACCG |
| | | CCAAGTACTTCTTCTACAGCAACATCATGAACTTTTTCAAG |
| | | ACCGAGATTACCCTGGCCAACGGCGAGATCCGGAAGCGGC |
| | | CTCTGATCGAGACAAACGGCGAAACCGGGGAGATCGTGTG |
| | | GGATAAGGGCCGGGATTTTGCCACCGTGCGGAAAGTGCTG |
| | | AGCATGCCCCAAGTGAATATCGTGAAAAAGACCGAGGTGC |
| | | AGACAGGCGGCTTCAGCAAAGAGTCTATCCTGCCCAAGAG |
| | | GAACAGCGATAAGCTGATCGCCAGAAAGAAGGACTGGGA |
| | | CCCTAAGAAGTACGGCGGCTTCGACAGCCCCACCGTGGCC |
| | | TATTCTGTGCTGGTGGTGGCCAAAGTGGAAAAGGGCAAGT |
| | | CCAAGAAACTGAAGAGTGTGAAAGAGCTGCTGGGGATCA |
| | | CCATCATGGAAAGAAGCAGCTTCGAGAAGAATCCCATCGA |
| | | CTTTCTGGAAGCCAAGGGCTACAAAGAAGTGAAAAAGGA |
| | | CCTGATCATCAAGCTGCCTAAGTACTCCCTGTTCGAGCTGG |
| | | AAAACGGCCGGAAGAGAATGCTGGCCTCTGCCGGCGAACT |
| | | GCAGAAGGGAAACGAACTGGCCCTGCCCTCCAAATATGTG |
| | | AACTTCCTGTACCTGGCCAGCCACTATGAGAAGCTGAAGG |
| | | GCTCCCCCGAGGATAATGAGCAGAAACAGCTGTTTGTGGA |
| | | ACAGCACAAGCACTACCTGGACGAGATCATCGAGCAGATC |
| | | AGCGAGTTCTCCAAGAGAGTGATCCTGGCCGACGCTAATC |
| | | TGGACAAAGTGCTGTCCGCCTACAACAAGCACCGGGATAA |
| | | GCCCATCAGAGAGCAGGCCGAGAATATCATCCACCTGTTT |
| | | ACCCTGACCAATCTGGGAGCCCCTGCCGCCTTCAAGTACTT |
| | | TGACACCACCATCGACCGGAAGAGGTACACCAGCACCAA |
| | | AGAGGTGCTGGACGCCACCCTGATCCACCAGAGCATCACC |
| | | GGCCTGTACGAGACACGGATCGACCTGTCTCAGCTGGGAG |
| | | GCGACAAAAGGCCGGCGGCCACGAAAAAGGCCGGccaggcaa |
| | | aaaagaaaaagggatcctaa |
| SEQ ID | spCas9 (D10A) | DKKYSIGLDIGTNSVGWAVITDEYKVPSKKFKVLGNTDRHSI |
| NO: 30 | protein sequence | KKNLIGALLFDSGETAEATRLKRTARRRYTRRKNRICYLQEIF |
| | | SNEMAKVDDSFFHRLEESFLVEEDKKHERHPIFGNIVDEVAY |
| | | HEKYPTIYHLRKKLVDSTDKADLRLIYLALAHMIKFRGHFLIE |
| | | GDLNPDNSDVDKLFIQLVQTYNQLFEENPINASGVDAKAILS |
| | | ARLSKSRRLENLIAQLPGEKKNGLFGNLIALSLGLTPNFKSNF |
| | | DLAEDAKLQLSKDTYDDDLDNLLAQIGDQYADLFLAAKNLS |
| | | DAILLSDILRVNTEITKAPLSASMIKRYDEHHQDLTLLKALVR |
| | | QQLPEKYKEIFFDQSKNGYAGYIDGGASQEEFYKFIKPILEKM |
| | | DGTEELLVKLNREDLLRKQRTFDNGSIPHQIHLGELHAILRRQ |
| | | EDFYPFLKDNREKIEKILTFRIPYYVGPLARGNSRFAWMTRKS |
| | | EETITPWNFEEVVDKGASAQSFIERMTNFDKNLPNEKVLPKH |
| | | SLLYEYFTVYNELTKVKYVTEGMRKPAFLSGEQKKAIVDLLF |
| | | KTNRKVTVKQLKEDYFKKIECFDSVEISGVEDRFNASLGTYH |
| | | DLLKIIKDKDFLDNEENEDILEDIVLTLTLFEDREMIEERLKTY |
| | | AHLFDDKVMKQLKRRRYTGWGRLSRKLINGIRDKQSGKTIL |
| | | DFLKSDGFANRNFMQLIHDDSLTFKEDIQKAQVSGQGDSLHE |
| | | HIANLAGSPAIKKGILQTVKVVDELVKVMGRHKPENIVIEMA |
| | | RENQTTQKGQKNSRERMKRIEEGIKELGSQILKEHPVENTQL |
| | | QNEKLYLYYLQNGRDMYVDQELDINRLSDYDVDHIVPQSFL |
| | | KDDSIDNKVLTRSDKNRGKSDNVPSEEVVKKMKNYWRQLL |
| | | NAKLITQRKFDNLTKAERGGLSELDKAGFIKRQLVETRQITK |
| | | HVAQILDSRMNTKYDENDKLIREVKVITLKSKLVSDFRKDFQ |
| | | FYKVREINNYHHAHDAYLNAVVGTALIKKYPKLESEFVYGD |
| | | YKVYDVRKMIAKSEQEIGKATAKYFFYSNIMNFFKTEITLAN |
| | | GEIRKRPLIETNGETGEIVWDKGRDFATVRKVLSMPQVNIVK |
| | | KTEVQTGGFSKESILPKRNSDKLIARKKDWDPKKYGGFDSPT |
| | | VAYSVLVVAKVEKGKSKKLKSVKELLGITIMERSSFEKNPIDF |
| | | LEAKGYKEVKKDLIIKLPKYSLFELENGRKRMLASAGELQKG |
| | | NELALPSKYVNFLYLASHYEKLKGSPEDNEQKQLFVEQHKH |
| | | YLDEIIEQISEFSKRVILADANLDKVLSAYNKHRDKPIREQAE |
| | | NIIHLFTLTNLGAPAAFKYFDTTIDRKRYTSTKEVLDATLIHQS |
| | | ITGLYETRIDLSQLGGD |
| SEQ ID | 2XHBB 3′UTR | Gctcgctttcttgctgtccaatttctattaaaggttcctttgttccctaagtccaactactaaactg |
| NO: 31 | | ggggatattatgaagggccttgagcatctggattctgcctaataaaaaacatttattttcattgct |
| | | agctcgctttcttgctgtccaatttctattaaaggttcctttgttccctaagtccaactactaaact |
| | | gggggatattatgaagggccttgagcatctggattctgcctaataaaaaacatttattttcattgc |
| SEQ ID | U6 promoter | Gagggcctatttcccatgattccttcatatttgcatatacgatacaaggctgttagagagataattg |
| NO: 32 | | gaattaatttgactgtaaacacaaagatattagtacaaaatacgtgacgtagaaagtaataatttct |
| | | tgggtagtttgcagttttaaaattatgttttaaaatggactatcatatgcttaccgtaacttgaaag |
| | | tatttcgatttcttggctttatatatcttgtggaaaggac |
| SEQ ID | sgRNA with MS2 | gtttgagagctaggccaacatgaggatcacccatgtctgcagggcctagcaagttcaaataaggc |
| NO: 33 | aptamer inserted | tagtccgttatcaacttggccaacatgaggatcacccatgtctgcagggccaagtggcaccgagt |
| | in tetraloop and an | cggtgc |
| | MS2 aptamer | |
| | inserted in stem | |
| | loop 2 (MS2 | |
| | aptamer | |
| | underlined) | |
| SEQ ID | CMV Promoter | CGTTACATAACTTACGGTAAATGGCCCGCCTGGCTGACCG |
| NO: 34 | | CCCAACGACCCCCGCCCATTGACGTCAATAGTAACGCCAA |
| | | TAGGGACTTTCCATTGACGTCAATGGGTGGAGTATTTACG |
| | | GTAAACTGCCCACTTGGCAGTACATCAAGTGTATCATATG |
| | | CCAAGTACGCCCCCTATTGACGTCAATGACGGTAAATGGC |
| | | CCGCCTGGCATTGTGCCCAGTACATGACCTTATGGGACTTT |
| | | CCTACTTGGCAGTACATCTACGTATTAGTCATCGCTATTAC |
| | | CATGGTCGAGGTGAGCCCCACGTTCTGCTTCACTCTCCCCA |
| | | TCTCCCCCCCCTCCCCACCCCCAATTTTGTATTTATTTATTT |
| | | TTTAATTATTTTGTGCAGCGATGGGGGCGGGGGGGGGGGG |
| | | GGGGCGCGCGCCAGGCGGGGCGGGGCGGGGCGAGGGGCG |
| | | GGGCGGGGCGAGGCGGAGAGGTGCGGCGGCAGCCAATCA |
| | | GAGCGGCGCGCTCCGAAAGTTTCCTTTTATGGCGAGGCGG |
| | | CGGCGGCGGCGGCCCTATAAAAAGCGAAGCGCGCGGCGG |
| | | GCG |
| SEQ ID | ABE-g1 sgRNA | GAACACAAAGCATAGACTGCgtttgagagctaggccaacatgaggatcaccc |
| NO: 35 | with MS2 aptamer | atgtctgcagggcctagcaagttcaaataaggctagtccgttatcaacttggccaacatgaggatc |
| | in tetraloop and | acccatgtctgcagggccaagtggcaccgagtcggtgc |
| | MS2 aptamer in | |
| | ST2 loop. (MS2 | |
| | aptamer | |
| | underlined; | |
| | genomic targeting | |
| | sequence | |
| | capitalized) | |
| SEQ ID | ABE-g2 sgRNA | GAGTATGAGGCATAGACTGCgtttgagagctaggccaacatgaggatcaccc |
| NO: 36 | with MS2 aptamer | atgtctgcagggcctagcaagttcaaataaggctagtccgttatcaacttggccaacatgaggatc |
| | in tetraloop and | acccatgtctgcagggccaagtggcaccgagtcggtgc |
| | MS2 aptamer in | |
| | ST2 loop. (MS2 | |
| | aptamer | |
| | underlined; | |
| | genomic targeting | |
| | sequence | |
| | capitalized) | |
| SEQ ID | ABE-g5 sgRNA | GATGAGATAATGATGAGTCAgtttgagagctaggccaacatgaggatcaccc |
| NO: 37 | with MS2 aptamer | atgtctgcagggcctagcaagttcaaataaggctagtccgttatcaacttggccaacatgaggatc |
| | in tetraloop and | acccatgtctgcagggccaagtggcaccgagtcggtgc |
| | MS2 aptamer in | |
| | ST2 loop. (MS2 | |
| | aptamer | |
| | underlined; | |
| | genomic targeting | |
| | sequence | |
| | capitalized) | |
| SEQ ID | sgRNA with com | GTTTGAGAGCTAggccCTGAATGCCTGCGAGCATCCCACggcc |
| NO: 38 | aptamer in | TAGCAAGTTCAAATAAGGCTAGTCCGTTATCAACTTGAAA |
| | tetraloop (com | AAGTGGCACCGAGTCGGTGC |
| | aptamer | |
| | underlined). | |
| SEQ ID | ABE-g1 sgRNA | GAACACAAAGCATAGACTGCGTTTGAGAGCTAggccCTGAATG |
| NO: 39 | with com aptamer | CCTGCGAGCATCCCACggccTAGCAAGTTCAAATAAGGCTA |
| | in tetraloop (com | GTCCGTTATCAACTTGAAAAAGTGGCACCGAGTCGGTGC |
| | aptamer | |
| | underlined; | |
| | targeting sequence | |
| | italicized). | |
| SEQ ID | ABE-g2 sgRNA | GAGTATGAGGCATAGACTGCGTTTGAGAGCTAggccCTGAAT |
| NO: 40 | with com aptamer | GCCTGCGAGCATCCCACggccTAGCAAGTTCAAATAAGGCT |
| | in tetraloop (com | AGTCCGTTATCAACTTGAAAAAGTGGCACCGAGTCGGTGC |
| | aptamer | |
| | underlined; | |
| | targeting sequence | |
| | italicized). | |
| SEQ ID | ABE-g5 sgRNA | GATGAGATAATGATGAGTCAGTTTGAGAGCTAggccCTGAATG |
| NO: 41 | with com aptamer | CCTGCGAGCATCCCACggccTAGCAAGTTCAAATAAGGCTA |
| | in tetraloop (com | GTCCGTTATCAACTTGAAAAAGTGGCACCGAGTCGGTGC |
| | aptamer | |
| | underlined; | |
| | targeting sequence | |
| | italicized). | |
| SEQ ID | Unmodified ABE- | GATGAGATAATGATGAGTCAGTTTGAGAGCTAgaaatagcaagttcaa |
| NO: 42 | g5 sgRNA | ataaggctagtccgttatcaacttgaaaaagtggcaccgagtcggtgc |
| | (targeting | |
| | sequence | |
| | italicized). | |
| SEQ ID | sgRNA with com | GTTTGAGAGCTAgaaatagcaagttcaaataaggctagtccgttatcaacttggCTG |
| NO 43 | aptamer in | AATGCCTGCGAGCATCCCACccAAGTGGCACCGAGTCGGTG |
| | tetraloop (com | C |
| | aptamer | |
| | underlined) | |
| SEQ ID | ABE-g1 sgRNA | GAACACAAAGCATAGACTGCGTTTGAGAGCTAgaaatagcaagttca |
| NO 44 | with com aptamer | aataaggctagtccgttatcaacttggCTGAATGCCTGCGAGCATCCCACcc |
| | in tetraloop (com | AAGTGGCACCGAGTCGGTGC |
| | aptamer | |
| | underlined; | |
| | targeting sequence | |
| | italicized) | |
| SEQ ID | ABE-g2 sgRNA | GAGTATGAGGCATAGACTGCGTTTGAGAGCTAgaaatagcaagttca |
| NO 45 | with com aptamer | aataaggctagtccgttatcaacttggCTGAATGCCTGCGAGCATCCCACcc |
| | in tetraloop (com | AAGTGGCACCGAGTCGGTGC |
| | aptamer | |
| | underlined; | |
| | targeting sequence | |
| | italicized) | |
| SEQ ID | ABE-g5 sgRNA | GATGAGATAATGATGAGTCAGTTTGAGAGCTAgaaatagcaagttcaa |
| NO 46 | with com aptamer | ataaggctagtccgttatcaacttggCTGAATGCCTGCGAGCATCCCACcc |
| | in tetraloop (com | AAGTGGCACCGAGTCGGTGC |
| | aptamer | |
| | underlined; | |
| | targeting sequence | |
| | italicized) | |
| SEQ ID | Unmodiifed | GTTTGAGAGCTAGAAATAGCAAGTTCAAATAAGGCTAGTC |
| NO: 47 | sgRNA from | CGTTATCAACTTGAAAAAGTGGCACCGAGTCGGTGC |
| | Plasmid No. 16 | |
| | (Table 1) | |
| SEQ ID | intron | GGAGTCGCTGCGACGCTGCCTTCGCCCCGTGCCCCGCTCCGCCGC |
| NO: 48 | | CGCCTCGCGCCGCCCGCCCCGGCTCTGACTGACCGCGTTACTCCC |
| | | ACAGGTGAGCGGGCGGGACGGCCCTTCTCCTCCGGGCTGTAATT |
| | | AGCTGAGCAAGAGGTAAGGGTTTAAGGGATGGTTGGTTGGTGGG |
| | | GTATTAATGTTTAATTACCTGGAGCACCTGCCTGAAATCACTTTT |
| | | TTTCAGGTTGGACCGGTGCCACC |

1. A mammalian expression plasmid comprising a eukaryotic promoter operably linked to a non-viral nucleic acid sequence, wherein the non-viral nucleic acid sequence comprises:
(i) a nucleic acid sequence encoding an adenosine base pair editor (ABE), wherein the ABE is a fusion protein comprising an adenosine deaminase and a catalytically impaired CRISPR-associated endonuclease; and
(ii) a guide RNA (gRNA) coding sequence,
wherein the gRNA coding sequence comprises at least one aptamer coding sequence.

2. The mammalian expression plasmid of claim 1, wherein the catalytically impaired CRISPR-associated endonuclease coding sequence encodes a Cas9 D10A protein.

3. The mammalian expression plasmid of claim 1, wherein the adenine base editor is ABE 7.10 or ABE8.

4. The mammalian expression plasmid of claim 1, wherein the at least one aptamer coding sequence encodes an aptamer sequence bound specifically by an ABP selected from the group consisting of MS2 coat protein, PP7 coat protein, lambda N RNA-binding domain, or Com protein.

5. The mammalian expression plasmid of claim 1, wherein the aptamer is an MS2 aptamer sequence or a com aptamer sequence.

6. The mammalian expression plasmid of claim 1, wherein the sgRNA coding sequence comprises at least one aptamer inserted into the tetraloop or the ST2 loop of the sgRNA coding sequence.

7. The mammalian expression plasmid of claim 6, wherein the sgRNA coding comprises at least one com aptamer inserted into the ST2 loop of the gRNA coding sequence.

8. A lentiviral packaging system comprising:
a) a packaging plasmid comprising a eukaryotic promoter operably linked to a Gag nucleotide sequence, wherein the Gag nucleotide sequence comprises a nucleocapsid (NC) coding sequence and a matrix protein (MA) coding sequence, wherein one or both of the NC coding sequence or the MA coding sequence comprises at least one non-viral aptamer-binding protein (ABP) nucleotide sequence, and wherein the packaging plasmid does not encode a functional integrase protein;
b) at least one mammalian expression plasmid of claim 1; and
c) an envelope plasmid comprising an envelope glycoprotein coding sequence.

9. The lentiviral packaging system of claim 8, wherein the packaging plasmid further comprises a Rev nucleotide sequence and a Tat nucleotide sequence.

10. The lentiviral packaging system of claim 8, further comprising a second packaging plasmid comprising a Rev nucleotide sequence.

11. The lentiviral packaging system of claim 8, wherein the at least one non-viral ABP nucleotide sequence encodes MS2 coat protein, PP7 coat protein, lambda N peptide, or Com protein.

12. A lentivirus-like particle comprising:
a) a fusion protein comprising a nucleocapsid (NC) protein or a matrix (MA) protein wherein the NC protein or MA protein comprises at least one non-viral aptamer binding protein (ABP); and
b) a ribonucleotide protein (RNP) complex comprising: (i) an adenine base editor (ABE), wherein the ABE is a fusion polypeptide comprising an adenine base editor and a catalytically impaired CRISPR-associated endonuclease; and (ii) a gRNA,
wherein the lentivirus-like particle does not comprise a functional integrase protein.

13. The lentivirus-like particle of claim 12, wherein the catalytically impaired CRISPR-associated endonuclease is a catalytically impaired Cas9 protein, a catalytically impaired Cpf1 protein, or a derivative of either.

14. The lentivirus-like particle of claim 12, wherein the adenine base editor is ABE 7.10 or ABE 8.

15. A method of producing a lentivirus-like particle, the method comprising:
a) transfecting a plurality of eukaryotic cells with the packaging plasmid, the at least one mammalian expression plasmid, and the envelope plasmid of the system of claim 8; and
b) culturing the transfected eukaryotic cells for sufficient time for lentivirus-like to be produced.

16. The method of claim 15, wherein the lentivirus-like particle comprises a ribonucleotide protein (RNP) complex comprising: (i) an adenine base editor (ABE), wherein the ABE is a fusion polypeptide comprising an adenosine deaminase and a catalytically impaired CRISPR-associated endonuclease; and (ii) a guide RNA.

17. The method of claim 16, wherein the plurality of eukaryotic cells are mammalian cells.

18. A lentivirus-like particle made by the method of claim 15.

19. A method of modifying a genomic target sequence in a cell, the method comprising transducing a plurality of eukaryotic cells with a plurality of viral particles, wherein the plurality of viral particles comprise a lentivirus-like particle according claim 12, wherein the RNP binds to the genomic target sequence in genomic DNA of the cell and the ABE deaminates an adenine at the genomic target sequence, thereby modifying the genomic target sequence.

20. The method of claim 19, wherein the plurality of eukaryotic cells are mammalian cells.

21. The method of claim 19, wherein the plurality of eukaryotic cells are cells present in subject.

22. The method of claim 21, wherein the subject is a human subject.

23. The method of claim 22, wherein the subject is injected with the plurality of viral particles.

24. A cell containing the plasmid of claim 1.

25. A cell containing the lentiviral packaging system of claim 8.

26. A cell containing the lentivirus-like particle of claim 12.

27. A cell modified using the method of claim 19.

28. A method for treating a disease in a subject comprising:
a) obtaining cells from the subject;
b) modifying the cells of the subject using the method of claim 19; and
c) administering the modified cells to the subject.

29. The method of claim 28, wherein the disease is cancer.

30. The method of claim 29, wherein the disease is sickle cell anemia.

31. The method of claim 28, wherein the cells are T cells.


SEQUENCE LISTING (139 sequences)

SEQ ID NO: 1 - DNA, 351 bp, Artificial Sequence
  Synthetic construct
atggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctac

SEQ ID NO: 2 - protein, 117 aa, Artificial Sequence
  Synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 3 - DNA, 363 bp, Artificial Sequence
  Synthetic construct
tccaaaacaatagtcctctccgtaggggaggcaacacggactttgaccgaaatccagtcaaccgctgaccgacaaatctttgaagagaaagtagggcctcttgtgggccgactgcgcttgactgcaagcttgcgacaaaacggcgcaaagactgcctatagggtcaaccttaaactcgaccaagccgacgtggtcgatagcggtctccctaaggttcggtatacgcaggtctggagtcatgacgtaacaatcgtagcaaacagcacagaagcctcccgaaaaagcctctacgatctgacgaaatccttggtggctacgtcacaggtggaagacctcgttgtcaaccttgtacctctgggtcga

SEQ ID NO: 4 - protein, 121 aa, Artificial Sequence
  Synthetic construct
SKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 5 - DNA, 66 bp, Artificial Sequence
  Synthetic construct
atggatgcacaaacacgccgccgcgaacgtcgcgcagagaaacaggctcaatggaaagcagcaaat

SEQ ID NO: 6 - protein, 22 aa, Artificial Sequence
  Synthetic construct
MDAQTRRRERRAEKQAQWKAAN

SEQ ID NO: 7 - DNA, 193 bp, Artificial Sequence
  Synthetic construct
atgaaatcaattcgctgtaaaaactgcaacaaactgttatttaaggcggattcctttgatcacattgaaatcaggtgtccgcgttgcaaacgtcacatcataatgctgaatgcctgcgagcatcccacggagaaacattgtgggaaaagagaaaaaatcacgcattctgacgaaaccgtgcgttattgagtat

SEQ ID NO: 8 - protein, 62 aa, Artificial Sequence
  Synthetic construct
MKSIRCKNCNKLLFKADSFDHIEIRCPRCKRHIIMLNACEHPTEKHCGKREKITHSDETVRY

SEQ ID NO: 9 - RNA, 19 nt, Artificial Sequence
  Synthetic construct
acaugaggaucacccaugu

SEQ ID NO: 10 - DNA, 19 bp, Artificial Sequence
  Synthetic construct
acatgaggatcacccatgt

SEQ ID NO: 11 - RNA, 25 nt, Artificial Sequence
  Synthetic construct
ggagcagacgauauggcgucgcucc

SEQ ID NO: 12 - DNA, 25 bp, Artificial Sequence
  Synthetic construct
ggagcagacgatatggcgtcgctcc

SEQ ID NO: 13 - RNA, 19 nt, Artificial Sequence
  Synthetic construct
gggcccugaagaagggccc

SEQ ID NO: 14 - DNA, 19 bp, Artificial Sequence
  Synthetic construct
gggccctgaagaagggccc

SEQ ID NO: 15 - RNA, 19 nt, Artificial Sequence
  Synthetic construct
cugaaugccugcgagcauc

SEQ ID NO: 16 - DNA, 18 bp, Artificial Sequence
  Synthetic construct
ctgaatgcctgcgagcat

SEQ ID NO: 17 - DNA, 132 bp, Artificial Sequence
  Synthetic construct
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 18 - RNA, 132 nt, Artificial Sequence
  Synthetic construct
gcucgcuuucuugcuguccaauuucuauuaaagguuccuuuguucccuaaguccaacuacuaaacugggggauauuaugaagggccuugagcaucuggauucugccuaauaaaaaacauuuauuuucauugc

SEQ ID NO: 19 - DNA, 165 bp, Artificial Sequence
  Synthetic construct
atacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaat

SEQ ID NO: 20 - protein, 55 aa, Artificial Sequence
  Synthetic construct
IQKGNFRNQRKTVKCFNCGKEGHIAKNCRAPRKKGCWKCGKEGHQMKDCTERQAN

SEQ ID NO: 21 - DNA, 396 bp, Artificial Sequence
  Synthetic construct
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattac

SEQ ID NO: 22 - protein, 131 aa, Artificial Sequence
  Synthetic construct
GARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY

SEQ ID NO: 23 - DNA, 291 bp, Artificial Sequence
  Synthetic construct
atggaacaagccccagaagaccagggaccgcagagggaaccatacaatgaatggacactagaacttttagaggaactcaagcgggaagcagtcagacactttcctagaccatggcttcatggcttaggacaacatatctatgaaacctatggagatacttggacgggggtggaagctataataagaattctgcaacgactactgtttgtccatttcagaattgggtgccagcatagccgaataggcattctaagacagagaagagcaagaaatggagccagtagatcctaa

SEQ ID NO: 24 - protein, 96 aa, Artificial Sequence
  Synthetic construct
MEQAPEDQGPQREPYNEWTLELLEELKREAVRHFPRPWLHGLGQHIYETYGDTWTGVEAIIRILQRLLFVHFRIGCQHSRIGILRQRRARNGASRS

SEQ ID NO: 25 - DNA, 618 bp, Artificial Sequence
  Synthetic construct
atgggttgcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataagctggaagaggccaataaaggagagaacaccagcttgttacaccctgtgagcctgcatggaatggatgaccctggaagagaagtgttagagtggaggtttgacagccgcctagcatttcatcacgtggcccgagagctgcatccggagtacttcaagaactgc

SEQ ID NO: 26 - protein, 206 aa, Artificial Sequence
  Synthetic construct
MGCKWSKSSVIGWPAVRERMRRAEPAADGVGAVSRDLEKHGAITSSNTAANNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKLEEANKGENTSLLHPVSLHGMDDPGREVLEWRFDSRLAFHHVARELHPEYFKNC

SEQ ID NO: 27 - DNA, 5403 bp, Artificial Sequence
  Synthetic construct
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagggatcctaa

SEQ ID NO: 28 - protein, 1800 aa, Artificial Sequence
  Synthetic construct
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGS

SEQ ID NO: 29 - DNA, 4278 bp, Artificial Sequence
  Synthetic construct
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagggatcctaa

SEQ ID NO: 30 - protein, 1367 aa, Artificial Sequence
  Synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 31 - DNA, 266 bp, Artificial Sequence
  Synthetic construct
gctcgctttcttgctgtccaatttctattaaaggttcctttgttccctaagtccaactactaaactgggggatattatgaagggccttgagcatctggattctgcctaataaaaaacatttattttcattgctagctcgctttcttgctgtccaatttctattaaaggttcctttgttccctaagtccaactactaaactgggggatattatgaagggccttgagcatctggattctgcctaataaaaaacatttattttcattgc

SEQ ID NO: 32 - DNA, 241 bp, Artificial Sequence
  Synthetic construct
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 33 - DNA, 136 bp, Artificial Sequence
  Synthetic construct
gtttgagagctaggccaacatgaggatcacccatgtctgcagggcctagcaagttcaaataaggctagtccgttatcaacttggccaacatgaggatcacccatgtctgcagggccaagtggcaccgagtcggtgc

SEQ ID NO: 34 - DNA, 565 bp, Artificial Sequence
  Synthetic construct
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 35 - DNA, 156 bp, Artificial Sequence
  Synthetic construct
gaacacaaagcatagactgcgtttgagagctaggccaacatgaggatcacccatgtctgcagggcctagcaagttcaaataaggctagtccgttatcaacttggccaacatgaggatcacccatgtctgcagggccaagtggcaccgagtcggtgc

SEQ ID NO: 36 - DNA, 156 bp, Artificial Sequence
  Synthetic construct
gagtatgaggcatagactgcgtttgagagctaggccaacatgaggatcacccatgtctgcagggcctagcaagttcaaataaggctagtccgttatcaacttggccaacatgaggatcacccatgtctgcagggccaagtggcaccgagtcggtgc

SEQ ID NO: 37 - DNA, 156 bp, Artificial Sequence
  Synthetic construct
gatgagataatgatgagtcagtttgagagctaggccaacatgaggatcacccatgtctgcagggcctagcaagttcaaataaggctagtccgttatcaacttggccaacatgaggatcacccatgtctgcagggccaagtggcaccgagtcggtgc

SEQ ID NO: 38 - DNA, 103 bp, Artificial Sequence
  Synthetic construct
gtttgagagctaggccctgaatgcctgcgagcatcccacggcctagcaagttcaaataaggctagtccgttatcaacttgaaaaagtggcaccgagtcggtgc

SEQ ID NO: 39 - DNA, 123 bp, Artificial Sequence
  Synthetic construct
gaacacaaagcatagactgcgtttgagagctaggccctgaatgcctgcgagcatcccacggcctagcaagttcaaataaggctagtccgttatcaacttgaaaaagtggcaccgagtcggtgc

SEQ ID NO: 40 - DNA, 123 bp, Artificial Sequence
  Synthetic construct
gagtatgaggcatagactgcgtttgagagctaggccctgaatgcctgcgagcatcccacggcctagcaagttcaaataaggctagtccgttatcaacttgaaaaagtggcaccgagtcggtgc

SEQ ID NO: 41 - DNA, 123 bp, Artificial Sequence
  Synthetic construct
gatgagataatgatgagtcagtttgagagctaggccctgaatgcctgcgagcatcccacggcctagcaagttcaaataaggctagtccgttatcaacttgaaaaagtggcaccgagtcggtgc

SEQ ID NO: 42 - DNA, 96 bp, Artificial Sequence
  Synthetic construct
gatgagataatgatgagtcagtttgagagctagaaatagcaagttcaaataaggctagtccgttatcaacttgaaaaagtggcaccgagtcggtgc

SEQ ID NO: 43 - DNA, 99 bp, Artificial Sequence
  Synthetic construct
gtttgagagctagaaatagcaagttcaaataaggctagtccgttatcaacttggctgaatgcctgcgagcatcccacccaagtggcaccgagtcggtgc

SEQ ID NO: 44 - DNA, 119 bp, Artificial Sequence
  Synthetic construct
gaacacaaagcatagactgcgtttgagagctagaaatagcaagttcaaataaggctagtccgttatcaacttggctgaatgcctgcgagcatcccacccaagtggcaccgagtcggtgc

SEQ ID NO: 45 - DNA, 119 bp, Artificial Sequence
  Synthetic construct
gagtatgaggcatagactgcgtttgagagctagaaatagcaagttcaaataaggctagtccgttatcaacttggctgaatgcctgcgagcatcccacccaagtggcaccgagtcggtgc

SEQ ID NO: 46 - DNA, 119 bp, Artificial Sequence
  Synthetic construct
gatgagataatgatgagtcagtttgagagctagaaatagcaagttcaaataaggctagtccgttatcaacttggctgaatgcctgcgagcatcccacccaagtggcaccgagtcggtgc

SEQ ID NO: 47 - DNA, 76 bp, Artificial Sequence
  Synthetic construct
gtttgagagctagaaatagcaagttcaaataaggctagtccgttatcaacttgaaaaagtggcaccgagtcggtgc

SEQ ID NO: 48 - DNA, 246 bp, Artificial Sequence
  Synthetic construct
ggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccacc

SEQ ID NO: 49 - DNA, 20 bp, Artificial Sequence
  Synthetic construct
atggccacagatttcaggag

SEQ ID NO: 50 - DNA, 20 bp, Artificial Sequence
  Synthetic construct
ggcttccactttttgtgagg

SEQ ID NO: 51 - DNA, 19 bp, Artificial Sequence
  Synthetic construct
aggcctcacacaagccttc

SEQ ID NO: 52 - DNA, 20 bp, Artificial Sequence
  Synthetic construct
acctggctgagctaactgtg

SEQ ID NO: 53 - DNA, 20 bp, Artificial Sequence
  Synthetic construct
tccagccccatctgtcaaac

SEQ ID NO: 54 - DNA, 22 bp, Artificial Sequence
  Synthetic construct
ggaacctcaggtgaaaagtcca

SEQ ID NO: 55 - DNA, 20 bp, Artificial Sequence
  Synthetic construct
acttcctgaaatgctgtgcg

SEQ ID NO: 56 - DNA, 20 bp, Artificial Sequence
  Synthetic construct
gtctgaggtcacacagtggg

SEQ ID NO: 57 - DNA, 20 bp, Artificial Sequence
  Synthetic construct
ctgagagcagggaccacatc

SEQ ID NO: 58 - DNA, 19 bp, Artificial Sequence
  Synthetic construct
cccgcagtctatgcttcgc

SEQ ID NO: 59 - DNA, 19 bp, Artificial Sequence
  Synthetic construct
cctgcagtctatgcctcac

SEQ ID NO: 60 - DNA, 21 bp, Artificial Sequence
  Synthetic construct
agccctgactcatcattaccc

SEQ ID NO: 61 - DNA, 20 bp, Artificial Sequence
  Synthetic construct
ttgcactgccattctaccaa

SEQ ID NO: 62 - DNA, 20 bp, Artificial Sequence
  Synthetic construct
atccacagcaacaccctctc

SEQ ID NO: 63 - DNA, 24 bp, Artificial Sequence
  Synthetic construct
accggatgagataatgatgagtca

SEQ ID NO: 64 - DNA, 20 bp, Artificial Sequence
  Synthetic construct
gcaccgactcggtgccactt

SEQ ID NO: 65 - DNA, 23 bp, Artificial Sequence
  Synthetic construct
gaacacaaagcatagactgcggg

SEQ ID NO: 66 - DNA, 23 bp, Artificial Sequence
  Synthetic construct
gagtatgaggcatagactgcagg

SEQ ID NO: 67 - DNA, 23 bp, Artificial Sequence
  Synthetic construct
gatgagataatgatgagtcaggg

SEQ ID NO: 68 - DNA, 24 bp, Artificial Sequence
  Synthetic construct
accggaacacaaagcatagactgc

SEQ ID NO: 69 - DNA, 24 bp, Artificial Sequence
  Synthetic construct
accggagtatgaggcatagactgc

SEQ ID NO: 70 - DNA, 24 bp, Artificial Sequence
  Synthetic construct
accggatgagataatgatgagtca

SEQ ID NO: 71 - DNA, 24 bp, Artificial Sequence
  Synthetic construct
aaacgcagtctatgctttgtgttc

SEQ ID NO: 72 - DNA, 24 bp, Artificial Sequence
  Synthetic construct
aaacgcagtctatgcctcatactc

SEQ ID NO: 73 - DNA, 24 bp, Artificial Sequence
  Synthetic construct
aaactgactcatcattatctcatc

SEQ ID NO: 74 - RNA, 36 nt, Artificial Sequence
  Synthetic construct
gaacacaaagcauagacugcguuuuagagcuaugcu

SEQ ID NO: 75 - RNA, 7 nt, Artificial Sequence
  Synthetic construct
cuacgaa

SEQ ID NO: 76 - DNA, 7 bp, Artificial Sequence
  Synthetic construct
ctacgaa

SEQ ID NO: 77 - DNA, 7 bp, Artificial Sequence
  Synthetic construct
  misc_feature (2)..(2): n is a, c, g, or t
cnacgaa

SEQ ID NO: 78 - DNA, 7 bp, Artificial Sequence
  Synthetic construct
  misc_feature (2)..(2): n is a, c, g, or t
cngcgaa

SEQ ID NO: 79 - DNA, 6 bp, Artificial Sequence
  Synthetic construct
  misc_feature (1)..(2): n is a, c, g, or t
nngrrt

SEQ ID NO: 80 - DNA, 6 bp, Artificial Sequence
  Synthetic construct
  misc_feature (1)..(2): n is a, c, g, or t
  misc_feature (6)..(6): n is a, c, g, or t
nngrrn

SEQ ID NO: 81 - RNA, 6 nt, Artificial Sequence
  Synthetic construct
aauaaa

SEQ ID NO: 82 - DNA, 20 bp, Artificial Sequence
  Synthetic construct
gcaccgactcggtgccactt

SEQ ID NO: 83 - DNA, 24 bp, Artificial Sequence
  Synthetic construct
accggaacacaaagcatagactgc

SEQ ID NO: 84 - DNA, 24 bp, Artificial Sequence
  Synthetic construct
aaacgcagtctatgctttgtgttc

SEQ ID NO: 85 - DNA, 24 bp, Artificial Sequence
  Synthetic construct
accggagtatgaggcatagactgc

SEQ ID NO: 86 - DNA, 24 bp, Artificial Sequence
  Synthetic construct
aaacgcagtctatgcctcatactc

SEQ ID NO: 87 - DNA, 24 bp, Artificial Sequence
  Synthetic construct
accggatgagataatgatgagtca

SEQ ID NO: 88 - DNA, 24 bp, Artificial Sequence
  Synthetic construct
aaactgactcatcattatctcatc

SEQ ID NO: 89 - DNA, 24 bp, Artificial Sequence
  Synthetic construct
accggaacacaaagcatagactgc

SEQ ID NO: 90 - DNA, 24 bp, Artificial Sequence
  Synthetic construct
aaacgcagtctatgctttgtgttc

SEQ ID NO: 91 - DNA, 24 bp, Artificial Sequence
  Synthetic construct
accggagtatgaggcatagactgc

SEQ ID NO: 92 - DNA, 24 bp, Artificial Sequence
  Synthetic construct
aaacgcagtctatgcctcatactc

SEQ ID NO: 93 - DNA, 24 bp, Artificial Sequence
  Synthetic construct
accggatgagataatgatgagtca

SEQ ID NO: 94 - DNA, 24 bp, Artificial Sequence
  Synthetic construct
aaactgactcatcattatctcatc

SEQ ID NO: 95 - DNA, 48 bp, Artificial Sequence
  Synthetic construct
aaggacgaaacaccggatgagataatgatgagtcagtttgagagctag

SEQ ID NO: 96 - DNA, 20 bp, Artificial Sequence
  Synthetic construct
ttatgtaacgggtaccaaaa

SEQ ID NO: 97 - DNA, 50 bp, Artificial Sequence
  Synthetic construct
aaggacgaaacaccggtgtcttcctcgaggaagacccgtttgagagctag

SEQ ID NO: 98 - DNA, 20 bp, Artificial Sequence
  Synthetic construct
ttatgtaacgggtaccaaaa

SEQ ID NO: 99 - DNA, 24 bp, Artificial Sequence
  Synthetic construct
accggaacacaaagcatagactgc

SEQ ID NO: 100 - DNA, 24 bp, Artificial Sequence
  Synthetic construct
aaacgcagtctatgctttgtgttc

SEQ ID NO: 101 - DNA, 24 bp, Artificial Sequence
  Synthetic construct
accggagtatgaggcatagactgc

SEQ ID NO: 102 - DNA, 24 bp, Artificial Sequence
  Synthetic construct
aaacgcagtctatgcctcatactc

SEQ ID NO: 103 - DNA, 48 bp, Artificial Sequence; identical to SEQ ID NO: 95
  Synthetic construct

SEQ ID NO: 104 - DNA, 20 bp, Artificial Sequence
  Synthetic construct
ttatgtaacgggtaccaaaa

SEQ ID NO: 105 - DNA, 48 bp, Artificial Sequence; identical to SEQ ID NO: 95
  Synthetic construct

SEQ ID NO: 106 - DNA, 20 bp, Artificial Sequence
  Synthetic construct
ttatgtaacgggtaccaaaa

SEQ ID NO: 107 - DNA, 13 bp, Artificial Sequence
  Synthetic construct
aaactacgaactc

SEQ ID NO: 108 - DNA, 68 bp, Artificial Sequence
  Synthetic construct
tttaatagaaaaaatgtttggaggaaaactacgaactcacatacgttgtttgaactgcaggagtacct

SEQ ID NO: 109 - DNA, 68 bp, Artificial Sequence
  Synthetic construct
tttaatagaaaaaatgtttggaggaaaactgcgaactcacatgcgttgtttgaactgcaggagtacct

SEQ ID NO: 110 - DNA, 68 bp, Artificial Sequence
  Synthetic construct
tttaatagaaaaaatgtttggaggaaaactgcgaactcacatacgttgtttgaactgcaggagtacct

SEQ ID NO: 111 - DNA, 68 bp, Artificial Sequence
  Synthetic construct
tttaatagaaaaaatgtttggaggaaaactacgaactcacatgcgttgtttgaactgcaggagtacct

SEQ ID NO: 112 - DNA, 68 bp, Artificial Sequence
  Synthetic construct
tttaatggaaaaaatgtttggaggaaaactacgaactcacatacgttgtttgaactgcaggagtacct

SEQ ID NO: 113 - DNA, 68 bp, Artificial Sequence
  Synthetic construct
tttaatggaaaaaatgtttggaggaaaactgcgaactcacatgcgttgtttgaactgcaggagtacct

SEQ ID NO: 114 - DNA, 68 bp, Artificial Sequence
  Synthetic construct
tttaatggaaaaaatgtttggaggaaaactgcgaactcacatacgttgtttgaactgcaggagtacct

SEQ ID NO: 115 - DNA, 68 bp, Artificial Sequence
  Synthetic construct
tttaatagaaaaaatgtttggaggaaaactacgaactcacatacgttgtttgaactgcaggagtgcct

SEQ ID NO: 116 - DNA, 68 bp, Artificial Sequence
  Synthetic construct
tttgatagaaaaaatgtttggaggaaaactgcgaactcacatgcgttgtttgaactgcaggagtacct

SEQ ID NO: 117 - DNA, 68 bp, Artificial sequence
  Synthetic construct
tttgatagaaaaaatgtttggaggaaaactgcgaactcacatacgttgtttgaactgcaggagtacct

SEQ ID NO: 118 - DNA, 40 bp, Artificial sequence
  Synthetic construct
aaggaaactggaacacaaagcatagactgcggggcgggcc

SEQ ID NO: 119 - DNA, 40 bp, Artificial sequence
  Synthetic construct
aaggaaactggaacgcaaagcatagactgcggggcgggcc

SEQ ID NO: 120 - DNA, 40 bp, Artificial sequence
  Synthetic construct
aaggaaactggaacgcgaagcatagactgcggggcgggcc

SEQ ID NO: 121 - RNA, 98 nt, Artificial sequence
  Synthetic construct
  misc_feature (1)..(20): n is a, c, g, or u
nnnnnnnnnnnnnnnnnnnnguuugagagcuagaaauagcaaguucaaauaaggcuaguccguuaucaacuugaaaaaguggcaccgagucggugcuu

SEQ ID NO: 122 - RNA, 34 nt, Artificial sequence
  Synthetic construct
ggccaacaugaggaucacccaugucugcagggcc

SEQ ID NO: 123 - RNA, 31 nt, Artificial sequence
  Synthetic construct
ggcccugaaugccugcgagcaucccacggcc

SEQ ID NO: 124 - DNA, 40 bp, Artificial sequence
  Synthetic construct
gagggtgagggatgagataatgatgagtcagggcttcagg

SEQ ID NO: 125 - DNA, 40 bp, Artificial sequence
  Synthetic construct
gagggtgagggatgaggtaatgatgagtcagggcttcagg

SEQ ID NO: 126 - DNA, 40 bp, Artificial sequence
  Synthetic construct
gagggtgagggatggggtaatgatgagtcagggcttcagg

SEQ ID NO: 127 - DNA, 40 bp, Artificial sequence
  Synthetic construct
gagggtgagggatggggtgatgatgagtcagggcttcagg

SEQ ID NO: 128 - DNA, 40 bp, Artificial sequence
  Synthetic construct
gagggtgagggatgaggtgatgatgagtcagggcttcagg

SEQ ID NO: 129 - DNA, 40 bp, Artificial sequence
  Synthetic construct
gagggtgagggatgggataatgatgagtcagggcttcagg

SEQ ID NO: 130 - DNA, 40 bp, Artificial sequence
  Synthetic construct
gagggtgagggatgagatgatgatgagtcagggcttcagg

SEQ ID NO: 131 - DNA, 40 bp, Artificial sequence
  Synthetic construct
gagggtgaggggtggggtaatgatgagtcagggcttcagg

SEQ ID NO: 132 - DNA, 40 bp, Artificial sequence
  Synthetic construct
gagggtgagggatgggatgatgatgagtcagggcttcagg

SEQ ID NO: 133 - DNA, 40 bp, Artificial sequence
  Synthetic construct
gagggtgaggggtgaggtaatgatgagtcagggcttcagg

SEQ ID NO: 134 - DNA, 40 bp, Artificial sequence; identical to SEQ ID NO: 118
  Synthetic construct

SEQ ID NO: 135 - DNA, 40 bp, Artificial sequence; identical to SEQ ID NO: 119
  Synthetic construct

SEQ ID NO: 136 - DNA, 40 bp, Artificial sequence; identical to SEQ ID NO: 120
  Synthetic construct

SEQ ID NO: 137 - DNA, 40 bp, Artificial sequence
  Synthetic construct
aaggaaactggagcgcaaagcatagactgcggggcgggcc

SEQ ID NO: 138 - DNA, 40 bp, Artificial sequence
  Synthetic construct
aaggaaactggaacacgaagcatagactgcggggcgggcc

SEQ ID NO: 139 - DNA, 40 bp, Artificial sequence
  Synthetic construct
aaggaaactggagcgcgaagcatagactgcggggcgggcc